SEQUENCE LISTING (102 sequences)

SEQ ID NO: 1 - DNA, 207 bp, Glycine max
  source 1..207: mol_type = unassigned DNA
cactctgtgaagactttactaacgtggtcactctagttgcctgtcatcaactattacaccacatccacacccttgttaacttaaagatgttattcccattctatcacattttggtaatctcttaacatatagaacactaccctgaacatagtaaagaagccagtgctaggatttgttagtgaagcaaatgaagtcttccacgaagtg

SEQ ID NO: 2 - DNA, 207 bp, Glycine max
  source 1..207: mol_type = unassigned DNA
cactctgtgaagactttactaacgtggtcactctagttgcctgtcatcaactattacaccacatccacacccttgttaacttaaagaaattattcccattctatcacattttggtaatctcttaacatatagaacactaccctgaacatagtaaagaagccagtgctaggatttgttagtgaagcaaatgaagtcttccacgaagtg

SEQ ID NO: 3 - DNA, 207 bp, Glycine max
  source 1..207: mol_type = unassigned DNA
cactctgtgaagactttactaacgtggtcactctagttgcctgtcatcaactattacaccacatccacacccttgttaacttaaagatgttattcccattctatcacattaaagtaatctcttaacatatagaacactaccctgaacatagtaaagaagccagtgctaggatttgttagtgaagcaaatgaagtcttccacgaagtg

SEQ ID NO: 4 - DNA, 583 bp, Glycine max
  source 1..583: mol_type = unassigned DNA
cactctgtgaagactttactccttattggactttagaatattgagtccatttgcaaggaaattggaagcaaatagaggcgtcgacgtgtctgttgaggttccagtgagtgacgggtctgtttgcagtccacacaaacaccatcatcatcatgttcttatctcatgttatgcgacggcgactcttctttcttcttcttcctcttcttcctcacattttcttattctatttctcattttacctgcacttggcctcttctacgccgcaaggcatgtttccaactttgctcattctaaattatatctctgcccttaactactattataaactaaattttgtaacttggctaatgaaaacaagtgggtatgcaatattcttggacaattatattttccttttattatagagtctttagtattccattttgggctatgattacctctgcattctaaatgatatcctgttgatcttgttgatttggttttcacagtatcaggttacatttggagttaagacatatacatatattaatatattattattattattatattatatgtatataatgaagtcttccacgaagtg

SEQ ID NO: 5 - DNA, 583 bp, Glycine max
  source 1..583: mol_type = unassigned DNA
cactctgtgaagactttactccttattggactttagaatattgagtccatttgcaaggaaattggaagcaaatagaggcgtcgacgtgtctgttgaggttccagtgagtgacgggtctgtttgcagtccacacaaacaccatcatcatcaaattcttatctcatgttatgcgacggcgactcttctttcttcttcttcctcttcttcctcacattttcttattctatttctcattttacctgcacttggcctcttctacgccgcaaggcatgtttccaactttgctcattctaaattatatctctgcccttaactactattataaactaaattttgtaacttggctaatgaaaacaagtgggtatgcaatattcttggacaattatattttccttttattatagagtctttagtattccattttgggctatgattacctctgcattctaaatgatatcctgttgatcttgttgatttggttttcacagtatcaggttacatttggagttaagacatatacatatattaatatattattattattattatattatatgtatataatgaagtcttccacgaagtg

SEQ ID NO: 6 - DNA, 583 bp, Glycine max
  source 1..583: mol_type = unassigned DNA
cactctgtgaagactttactccttattggactttagaatattgagtccatttgcaaggaaattggaagcaaatagaggcgtcgacgtgtctgttgaggttccagtgagtgacgggtctgtttgcagtccacacaaacaccatcatcatcatgttcttatctcaaattatgcgacggcgactcttctttcttcttcttcctcttcttcctcacattttcttattctatttctcattttacctgcacttggcctcttctacgccgcaaggcatgtttccaactttgctcattctaaattatatctctgcccttaactactattataaactaaattttgtaacttggctaatgaaaacaagtgggtatgcaatattcttggacaattatattttccttttattatagagtctttagtattccattttgggctatgattacctctgcattctaaatgatatcctgttgatcttgttgatttggttttcacagtatcaggttacatttggagttaagacatatacatatattaatatattattattattattatattatatgtatataatgaagtcttccacgaagtg

SEQ ID NO: 7 - DNA, 583 bp, Glycine max
  source 1..583: mol_type = unassigned DNA
cactctgtgaagactttactccttattggactttagaatattgagtccatttgcaaggaaattggaagcaaatagaggcgtcgacgtgtctgttgaggttccagtgagtgacgggtctgtttgcagtccacacaaacaccatcatcatcatgttcttatctcatgttaaacgacggcgactcttctttcttcttcttcctcttcttcctcacattttcttattctatttctcattttacctgcacttggcctcttctacgccgcaaggcatgtttccaactttgctcattctaaattatatctctgcccttaactactattataaactaaattttgtaacttggctaatgaaaacaagtgggtatgcaatattcttggacaattatattttccttttattatagagtctttagtattccattttgggctatgattacctctgcattctaaatgatatcctgttgatcttgttgatttggttttcacagtatcaggttacatttggagttaagacatatacatatattaatatattattattattattatattatatgtatataatgaagtcttccacgaagtg

SEQ ID NO: 8 - DNA, 306 bp, Glycine max
  source 1..306: mol_type = unassigned DNA
cactctgtgaagactttactgagttcacccctcaaacgttaattaattacaaggcttatgtctattttcagtatttttgacatcttcgttctcctgatttttgttggctttaactttaaaccaccaattctgttccccttgtttcttcatcattagattttaagtgccatgcattgatataatataaatatataacttcatttaattaatgcaagctattgagttttgagtcgaatctattagccacaaaacacacacacacatattctcacacaaactgcaaccaatgaagtcttccacgaagtg

SEQ ID NO: 9 - DNA, 306 bp, Glycine max
  source 1..306: mol_type = unassigned DNA
cactctgtgaagactttactgagttcacccctcaaacgttaattaattacaaggcttatgtctattttcagtatttttgacatcttcgttctccccatttttgttggctttaactttaaaccaccaattctgttccccttgtttcttcatcattagattttaagtgccatgcattgatataatataaatatataacttcatttaattaatgcaagctattgagttttgagtcgaatctattagccacaaaacacacacacacatattctcacacaaactgcaaccaatgaagtcttccacgaagtg

SEQ ID NO: 10 - DNA, 306 bp, Glycine max
  source 1..306: mol_type = unassigned DNA
cactctgtgaagactttactgagttcacccctcaaacgttaattaattacaaggcttatgtctattttcagtatttttgacatcttcgttctcctgatttttgttggctttaactttaaaccaccaattctgttccccttgtttcttcatcattagattttaagtgccatgcattgatataatataaatatataacttcatttaattaatgcaagctattgagttttgagtcgaatctattagccacaaaacacacacacacatattctcacacaaaccccaaccaatgaagtcttccacgaagtg

SEQ ID NO: 11 - DNA, 306 bp, Glycine max
  source 1..306: mol_type = unassigned DNA
cactctgtgaagactttactgagttcacccctcaaacgttaattaattacaaggcttatgtctattttcagtattttttacatcttcgttctcctgatttttgttggctttaactttaaaccaccaattctgttccccttgtttcttcatcattagattttaagtgccatgcattgatataatataaatatataacttcatttaattaatgcaagctattgagttttgagtcgaatctattagccacaaaacacacacacacatattctcacacaaactgcaaccaatgaagtcttccacgaagtg

SEQ ID NO: 12 - DNA, 383 bp, Vigna unguiculata
  source 1..383: mol_type = unassigned DNA
cactctgtgaagactttactaataaaattgaaccaagaaaataaaatacagagagcgaaatgaactaaagtgagtgagtgtactaagtttgaggcaggaaacgaaaggagatgaatattattttttgcaaaacctgaaatgaaaacattatcaccataccacaaatagtgttggagacgtggtcactcttgttgcctgtcatcaactattacaccacatccacacccttgttaaattatagatggtgttcctcttctatcatcattttggtaaaccctaccttaacatattcaacactaccattcccaacattctgaaccaaaccaccgaggtaaggtaacacttcgtttcatactccaccaaatgaagtcttccacgaagtg

SEQ ID NO: 13 - DNA, 383 bp, Vigna unguiculata
  source 1..383: mol_type = unassigned DNA
cactctgtgaagactttactaataaaattgaaccaagaaaataaaatacagagagcgaaatgaactaaagtgagtgagtgtactaagtttgaggcaggaaacgaaaggagatgaatattattttttgcaaaacctgaaatgaaaacattatcaccataccacaaatagtgttggagacgtggtcactcttgttgcctgtcatcaactattacaccacatccacacccttgttaaattatagaaagtgttcctcttctatcatcattttggtaaaccctaccttaacatattcaacactaccattcccaacattctgaaccaaaccaccgaggtaaggtaacacttcgtttcatactccaccaaatgaagtcttccacgaagtg

SEQ ID NO: 14 - DNA, 383 bp, Vigna unguiculata
  source 1..383: mol_type = unassigned DNA
cactctgtgaagactttactaataaaattgaaccaagaaaataaaatacagagagcgaaatgaactaaagtgagtgagtgtactaagtttgaggcaggaaacgaaaggagatgaatattattttttgcaaaacctgaaatgaaaacattatcaccataccacaaatagtgtttgagacgtggtcactcttgttgcctgtcatcaactattacaccacatccacacccttgttaaattatagatggtgttcctcttctatcatcattttggtaaaccctaccttaacatattcaacactaccattcccaacattctgaaccaaaccaccgaggtaaggtaacacttcgtttcatactccaccaaatgaagtcttccacgaagtg

SEQ ID NO: 15 - DNA, 383 bp, Vigna unguiculata
  source 1..383: mol_type = unassigned DNA
cactctgtgaagactttactaataaaattgaaccaagaaaataaaatacagagagcgaaatgaactaaagtgagtgagtgtactaagtttgaggcaggaaacgaaaggagatgaatattattttttgcaaaacctgaaatgaaaacattatcaccataccacaaatagtgttggagacgtggtcactcttgttgcctgtcatcaactattacaccacatccacacccttgttaaattatagatggtgttcctcttctatcatcatttttgtaaaccctaccttaacatattcaacactaccattcccaacattctgaaccaaaccaccgaggtaaggtaacacttcgtttcatactccaccaaatgaagtcttccacgaagtg

SEQ ID NO: 16 - DNA, 440 bp, Vigna unguiculata
  source 1..440: mol_type = unassigned DNA
cactctgtgaagactttactactctatgggtttgaatcctgcttcagattttcctacgtgggcgagcttacaacatacacaccactcccatccatgaccacaattaaggtaccaaaatttgtctgtcactgtcacccaacactagcaaccactgatctcatttctcacacgttaattacaaattacgaaccttacgtctagtttcatcatcctcattctcctgatttttctaaccattaactttaaaccactgattctgttgtccttgtttattcgtcattagattttgttatttcgattcattgtcaaaacttcaacctgtaatcgtataaatataacttcacctgattaatgcaacctagtttttgagttttgagttgagccacacaaggcacacattcacacacgcaccaacaaccaatgaagtcttccacgaagtg

SEQ ID NO: 17 - DNA, 440 bp, Vigna unguiculata
  source 1..440: mol_type = unassigned DNA
cactctgtgaagactttactactctaaaggtttgaatcctgcttcagattttcctacgtgggcgagcttacaacatacacaccactcccatccatgaccacaattaaggtaccaaaatttgtctgtcactgtcacccaacactagcaaccactgatctcatttctcacacgttaattacaaattacgaaccttacgtctagtttcatcatcctcattctcctgatttttctaaccattaactttaaaccactgattctgttgtccttgtttattcgtcattagattttgttatttcgattcattgtcaaaacttcaacctgtaatcgtataaatataacttcacctgattaatgcaacctagtttttgagttttgagttgagccacacaaggcacacattcacacacgcaccaacaaccaatgaagtcttccacgaagtg

SEQ ID NO: 18 - DNA, 440 bp, Vigna unguiculata
  source 1..440: mol_type = unassigned DNA
cactctgtgaagactttactactctatgggtttgaatcctgcttcagattttcctacgtgggcgagcttacaacatacacaccactcccatccaaaaccacaattaaggtaccaaaatttgtctgtcactgtcacccaacactagcaaccactgatctcatttctcacacgttaattacaaattacgaaccttacgtctagtttcatcatcctcattctcctgatttttctaaccattaactttaaaccactgattctgttgtccttgtttattcgtcattagattttgttatttcgattcattgtcaaaacttcaacctgtaatcgtataaatataacttcacctgattaatgcaacctagtttttgagttttgagttgagccacacaaggcacacattcacacacgcaccaacaaccaatgaagtcttccacgaagtg

SEQ ID NO: 19 - DNA, 440 bp, Vigna unguiculata
  source 1..440: mol_type = unassigned DNA
cactctgtgaagactttactactctatgggtttgaatcctgcttcagattttcctacgtgggcgagcttacaacatacacaccactcccatccatgaccacaattaaggtaccaaaatttgtctgtcactgtcacccaacactagcaaccactgatctcatttctcacacgttaattacaaattacgaaccttacgtctagtttcatcatcctcattctcctgatttttctaaccattaactttaaaccactgattctgttgtccttgtttattcgtcattagattttgttatttcgattcattgtcaaaacttcaacctgtaatcgtataaatataacttcacctgattaaaacaacctagtttttgagttttgagttgagccacacaaggcacacattcacacacgcaccaacaaccaatgaagtcttccacgaagtg

SEQ ID NO: 20 - DNA, 205 bp, Arabidopsis thaliana
  source 1..205: mol_type = unassigned DNA
  misc_feature 106..127: note = uORF
cactctgtgaagactttactgtcttttgactctcttctctctctcctctctttctgctttcctcaatctctctctttctatctctagagcttccacttcctctctaatggtggaaccaaaaccctagattcccctttcatcttctctacttcccacacttttctctctcacaaactcttgagaaaatgaagtcttccacgaagtg

SEQ ID NO: 21 - DNA, 548 bp, Lactuca sativa
  source 1..548: mol_type = unassigned DNA
  misc_feature 218..410: note = uORF
cactctgtgaagactttactaaccacttttccaccattcattcgcatcatcaatctcattcaaagtttcaaacaaatcaccctcttcccctttgctgattgacctactgctgccggataatcgtcatcgaattaggagaaacagagtctacccttggggttgcctgctagagaggaacgggttggggtaaaaccctggaacgacaagttgcagacatcacggctatacacggagcaacgcggccactcgttcatgtccgatcagtacggcggaagggttcaataactattgagagcaacccttcgacgcacggcggacgtggcgctttgccttccgaaggcggtagcccatccgaccttctcttcctcgccggcggtggttcatcctccttcctattcctcgtagcttagggtatattaggtctagacttatacgcatatatcccatacctgttattatcgtttaattttcaagatctgctttcttcatcgctcgtatccgtttaatcaaccaaccaatcgaagaacaatgaagtcttccacgaagtg

SEQ ID NO: 22 - DNA, 166 bp, Glycine max
  source 1..166: mol_type = unassigned DNA
  misc_feature 67..69: note = uORF start codon
  misc_feature 153..155: note = uORF start codon
  misc_feature 91..93: note = uORF start codon
aacgtggtcactctagttgcctgtcatcaactattacaccacatccacacccttgttaacttaaagatgttattcccattctatcacattttggtaatctcttaacatatagaacactaccctgaacatagtaaagaagccagtgctaggatttgttagtgaagca

SEQ ID NO: 23 - DNA, 542 bp, Glycine max
  source 1..542: mol_type = unassigned DNA
  misc_feature 130..132: note = uORF start codon
  misc_feature 143..145: note = uORF start codon
  misc_feature 148..150: note = uORF start codon
ccttattggactttagaatattgagtccatttgcaaggaaattggaagcaaatagaggcgtcgacgtgtctgttgaggttccagtgagtgacgggtctgtttgcagtccacacaaacaccatcatcatcatgttcttatctcatgttatgcgacggcgactcttctttcttcttcttcctcttcttcctcacattttcttattctatttctcattttacctgcacttggcctcttctacgccgcaaggcatgtttccaactttgctcattctaaattatatctctgcccttaactactattataaactaaattttgtaacttggctaatgaaaacaagtgggtatgcaatattcttggacaattatattttccttttattatagagtctttagtattccattttgggctatgattacctctgcattctaaatgatatcctgttgatcttgttgatttggttttcacagtatcaggttacatttggagttaagacatatacatatattaatatattattattattattatattatatgtatat

SEQ ID NO: 24 - DNA, 265 bp, Glycine max
  source 1..265: mol_type = unassigned DNA
  misc_feature 57..59: note = uORF start codon
  misc_feature 74..76: note = uORF start codon
  misc_feature 258..260: note = uORF start codon
gagttcacccctcaaacgttaattaattacaaggcttatgtctattttcagtatttttgacatcttcgttctcctgatttttgttggctttaactttaaaccaccaattctgttccccttgtttcttcatcattagattttaagtgccatgcattgatataatataaatatataacttcatttaattaatgcaagctattgagttttgagtcgaatctattagccacaaaacacacacacacatattctcacacaaactgcaacc

SEQ ID NO: 25 - DNA, 342 bp, Vigna unguiculata
  source 1..342: mol_type = unassigned DNA
  misc_feature 151..153: note = uORF start codon
  misc_feature 222..224: note = uORF start codon
  misc_feature 248..250: note = uORF start codon
aataaaattgaaccaagaaaataaaatacagagagcgaaatgaactaaagtgagtgagtgtactaagtttgaggcaggaaacgaaaggagatgaatattattttttgcaaaacctgaaatgaaaacattatcaccataccacaaatagtgttggagacgtggtcactcttgttgcctgtcatcaactattacaccacatccacacccttgttaaattatagatggtgttcctcttctatcatcattttggtaaaccctaccttaacatattcaacactaccattcccaacattctgaaccaaaccaccgaggtaaggtaacacttcgtttcatactccacca

SEQ ID NO: 26 - DNA, 399 bp, Vigna unguiculata
  source 1..399: mol_type = unassigned DNA
  misc_feature 6..8: note = uORF start codon
  misc_feature 74..76: note = uORF start codon
  misc_feature 332..334: note = uORF start codon
actctatgggtttgaatcctgcttcagattttcctacgtgggcgagcttacaacatacacaccactcccatccatgaccacaattaaggtaccaaaatttgtctgtcactgtcacccaacactagcaaccactgatctcatttctcacacgttaattacaaattacgaaccttacgtctagtttcatcatcctcattctcctgatttttctaaccattaactttaaaccactgattctgttgtccttgtttattcgtcattagattttgttatttcgattcattgtcaaaacttcaacctgtaatcgtataaatataacttcacctgattaatgcaacctagtttttgagttttgagttgagccacacaaggcacacattcacacacgcaccaacaacc

SEQ ID NO: 27 - DNA, 1059 bp, Arabidopsis thaliana
  source 1..1059: mol_type = unassigned DNA
atcttttaatctcttcacttccacaacacaacacaaaacacatcactgatccttctctcatcctcagaaagaatggctcaaaccatgctgcttacttcaggcgtcaccgccggccattttttgaggaacaagagccctttggctcagcccaaagttcaccatctcttcctctctggaaactctccggttgcactaccatctaggagacaatcattcgttcctctcgctctcttcaaacccaaaaccaaagctgctcctaaaaaggttgagaagccgaagagcaaggttgaggatggcatctttggaacgtctggtgggattggtttcacaaaggcgaatgagctattcgttggtcgtgttgctatgatcggtttcgctgcatcgttgcttggtgaggcgttgacgggaaaagggatattagctcagctgaatctggagacagggataccgatttacgaagcagagccattgcttctcttcttcatcttgttcactctgttgggagccattggagctctcggagacagaggaaaattcgtcgacgatcctcccaccgggctcgagaaagccgtcattcctcccggcaaaaacgtccgatctgccctcggtctcaaagaacaaggtccattgtttgggttcacgaaggcgaacgagttattcgtaggaagattggcacagttgggaatagcattttcactgataggagagattattaccgggaaaggagcattagctcaactcaacattgagaccggtataccaattcaagatatcgaaccacttgtcctcttaaacgttgctttcttcttcttcgctgccattaatcctggtaatggaaaattcatcaccgatgatggtgaagaaagctaaattatcatgtacttaaatttagtagagagtgtgtgaccttctcttcatgttgagacaaaaggaaatggacagcttaaattgttgtaatacttatatccttttgtttttaacttggaattttctgattatatatagttttattacacctaagtgactaggtgattgatatcatctagcatctcttaacag

SEQ ID NO: 28 - DNA, 2385 bp, Arabidopsis thaliana
  source 1..2385: mol_type = unassigned DNA
atttgcctccttcttcttcaaaccaatttcttcttcttcttctctcaggtttgagtctgaggcatcacaacagataaagtcttgaactttgatcaagaattctaaacggagtttttgctcttctcgtcggtgaaagaaaaatcgagatgggttcaactccgttttgctactctatcaatccatctccatcaaagcttgatttcacgaggacccatgtgtttagtcctgtttctaaacagttttacttagatttatcatcgttttccggaaaacccggaggagtatctgggtttaggagccgtcgagctttgctcggagtaaaggcggcgacggcgttagttgagaaggaggagaagagagaggcggtgacggagaagaagaagaaatcgagggttttagttgccggaggtggaatcggaggattggtgtttgctttagcggctaagaagaaaggattcgatgtgttagtgtttgagaaagatttgagtgctataagaggagaaggaaaatacagaggcccgattcaaatacagagcaacgctttagctgctttggaagctattgatattgaagttgctgaacaagttatggaagctgggtgtatcactggtgatcggattaacggtctcgttgatggtatctctggtacttggtatgtaaagtttgatactttcactcctgcggcgtcacggggacttcctgtgactagagtaattagtagaatgactctgcagcagattctagcacgtgcggttggagaagatgtgattagaaacgagagtaatgttgttgattttgaagattctggagataaggttactgtggtactcgagaatggtcaacgctatgaaggtgatctgcttgtgggtgcagatggcatttggtctaaggtgagaaataatttgtttggccgtagtgaagctacttattcaggctacacttgttacacggggattgcagattttataccagcggatatcgagtctgttggctaccgggttttcttgggacacaaacagtactttgtttcttcggatgttggtggtggaaaaatgcaatggtatgcatttcacgaggaaccagctggtggggctgatgctccaaatggtatgaagaaaaggttgtttgaaatatttgacggttggtgcgacaatgtactcgacttgttgcatgcgactgaggaggaagccattctgagaagagatatttatgatagaagtcctggttttacttggggtaaagggcgtgttacgctgctcggggattctatccatgcgatgcagccaaatatgggtcaaggtggatgcatggccattgaggatagttttcaactagcattggagcttgatgaagcatggaaacagagtgttgaaacgactacacctgttgatgttgtttcctctttgaaaagatatgaggaatctagaagactgagagtcgctattatccatgcaatggcgaggatggctgcaattatggcttccacttacaaagcatacttaggtgttgggcttggtcctctgtctttcttgacaaagtttagagtaccacatccaggaagagttggtggtagattcttcgttgacattgctatgccatcgatgcttgactgggtccttggaggtaacagtgaaaaactccaaggaaggccacctagttgcagactcactgacaaagccgatgaccggcttcgagagtggtttgaagatgacgatgctcttgaacgtactataaagggagaatggtatctaattccacacggcgacgattgttgcgtttcggaaacattatgtctaaccaaagatgaagatcaaccttgcatcgtcggaagcgaaccagatcaagattttcctggaatgcgcattgtgatcccttcgtctcaggtttcgaagatgcatgctcgtgtgatttacaaagacggagctttcttcttgatggatcttcgaagcgaacacggaacctatgtgaccgataacgaaggaagaagatatagagcaacaccgaattttcccgcgcggtttagatcgtccgacatcatcgagtttggttcagataagaaggcggcgtttagggtgaaagtaatcaggaaaactccgaaatcgacgaggaagaatgagagtaacaacgataaattacttcagacagcttgaaataagtaaaccgatggtgaaattaagtaattttaatcggtttggcagataatttgagtagtaattttctcaaaagaaaaaaaatctgctttcagctattccttgcatgacaatgtatatataggtctgaaacaaaatataaattatacaagacattaatcttcatttcttcaacttccaggcaacaataatctaattttgattactaaaaggtaattatattatcaaattgttt

SEQ ID NO: 29 - DNA, 1776 bp, Arabidopsis thaliana
  source 1..1776: mol_type = unassigned DNA
gagatatttgggtggagaaaacaaccgcctaggacaaaaaaaaattgtgggtagaggaggaggaggaggctttgttcacacaccacacgacggaacattctcttccgagtcttatccaaagcaaaatcatattttgcttaaatatcttcttctccggctctgtctctgtctctcttcttcccgcggagctttcagtttttgtttggtattgcttggtgtggggaagattagatagtgtgaagaatggcagtagctacacattgtttcacttcaccttgtcatgaccgtattcgatttttctcaagtgatgatggtattggtaggcttggcattacaagaaagaggatcaatggcactttcttgctcaagattttacctccaatccaaagtgctgatctcagaacaactggtgggagatcctcacgtcctttatctgcattcaggtcaggattctctaaggggatatttgacattgtgccattaccatcaaagaatgagctgaaagagctgaccgctccgctgttgctaaaactcgtgggtgttttagcttgcgcgttccttattgttccatctgcagatgcagttgatgcacttaaaacttgtgcatgcttattgaagggatgcaggatagaactcgcaaagtgcattgccaaccctgcctgtgcagccaatgtcgcgtgccttcagacctgcaataaccgtccagatgaaaccgagtgccagattaaatgtggggatctgtttgagaacagtgttgttgatgagttcaacgagtgtgctgtgtcgagaaaaaagtgtgttcctagaaaatctgatctcggagaatttcctgccccagacccttctgttcttgtacagaacttcaacatctcggactttaacgggaagtggtacattacaagtggcttgaatccaacctttgatgccttcgactgccagctgcatgagttccacacagaaggtgacaacaagcttgttggaaacatctcttggagaataaagaccctagacagtggattctttactaggtcagccgtacaaaaattcgtgcaagatcctaaccaacctggtgttctctacaatcatgacaacgagtaccttcactatcaagatgactggtatatcctgtcatcaaagatagagaataaacctgaagactatatatttgtatactaccgtgggcgaaacgatgcttgggatggatatggtggtgcagttgtatacacgagaagttctgtattacccaatagcattataccagaactcgaaaaagcagcaaaaagcataggcagagacttcagcacattcattagaacggataacacatgtggtcctgaacctgcgctcgtggagagaattgagaagacagtggaagaaggtgaaaggataatcgtaaaagaggttgaagagatagaagaagaggtagagaaggaagtggagaaggtcggtaggactgagatgaccttgttccagagattggctgaaggatttaatgaactgaagcaagacgaggagaatttcgtgagagagttaagtaaagaagagatggagtttttggatgagatcaaaatggaagcaagtgaggttgaaaaattgtttgggaaagctttgccaatcaggaaggtcaggtagaaacaagaaccaccattgttgtacaaactatattatacatactgtgttcggttcatataaagtaatatttttgtacacagtcatcatcattccataacaattggatacagaaaacacaaggattaaacatcctttgagcgaaac

SEQ ID NO: 30 - protein, 265 aa, Arabidopsis thaliana
MAQTMLLTSGVTAGHFLRNKSPLAQPKVHHLFLSGNSPVALPSRRQSFVPLALFKPKTKAAPKKVEKPKSKVEDGIFGTSGGIGFTKANELFVGRVAMIGFAASLLGEALTGKGILAQLNLETGIPIYEAEPLLLFFILFTLLGAIGALGDRGKFVDDPPTGLEKAVIPPGKNVRSALGLKEQGPLFGFTKANELFVGRLAQLGIAFSLIGEIITGKGALAQLNIETGIPIQDIEPLVLLNVAFFFFAAINPGNGKFITDDGEES

SEQ ID NO: 31 - protein, 667 aa, Arabidopsis thaliana
MGSTPFCYSINPSPSKLDFTRTHVFSPVSKQFYLDLSSFSGKPGGVSGFRSRRALLGVKAATALVEKEEKREAVTEKKKKSRVLVAGGGIGGLVFALAAKKKGFDVLVFEKDLSAIRGEGKYRGPIQIQSNALAALEAIDIEVAEQVMEAGCITGDRINGLVDGISGTWYVKFDTFTPAASRGLPVTRVISRMTLQQILARAVGEDVIRNESNVVDFEDSGDKVTVVLENGQRYEGDLLVGADGIWSKVRNNLFGRSEATYSGYTCYTGIADFIPADIESVGYRVFLGHKQYFVSSDVGGGKMQWYAFHEEPAGGADAPNGMKKRLFEIFDGWCDNVLDLLHATEEEAILRRDIYDRSPGFTWGKGRVTLLGDSIHAMQPNMGQGGCMAIEDSFQLALELDEAWKQSVETTTPVDVVSSLKRYEESRRLRVAIIHAMARMAAIMASTYKAYLGVGLGPLSFLTKFRVPHPGRVGGRFFVDIAMPSMLDWVLGGNSEKLQGRPPSCRLTDKADDRLREWFEDDDALERTIKGEWYLIPHGDDCCVSETLCLTKDEDQPCIVGSEPDQDFPGMRIVIPSSQVSKMHARVIYKDGAFFLMDLRSEHGTYVTDNEGRRYRATPNFPARFRSSDIIEFGSDKKAAFRVKVIRKTPKSTRKNESNNDKLLQTA

SEQ ID NO: 32 - protein, 462 aa, Arabidopsis thaliana
MAVATHCFTSPCHDRIRFFSSDDGIGRLGITRKRINGTFLLKILPPIQSADLRTTGGRSSRPLSAFRSGFSKGIFDIVPLPSKNELKELTAPLLLKLVGVLACAFLIVPSADAVDALKTCACLLKGCRIELAKCIANPACAANVACLQTCNNRPDETECQIKCGDLFENSVVDEFNECAVSRKKCVPRKSDLGEFPAPDPSVLVQNFNISDFNGKWYITSGLNPTFDAFDCQLHEFHTEGDNKLVGNISWRIKTLDSGFFTRSAVQKFVQDPNQPGVLYNHDNEYLHYQDDWYILSSKIENKPEDYIFVYYRGRNDAWDGYGGAVVYTRSSVLPNSIIPELEKAAKSIGRDFSTFIRTDNTCGPEPALVERIEKTVEEGERIIVKEVEEIEEEVEKEVEKVGRTEMTLFQRLAEGFNELKQDEENFVRELSKEEMEFLDEIKMEASEVEKLFGKALPIRKVR

SEQ ID NO: 33 - protein, 272 aa, Cucumis melo
MAAQTMLLTSGVCVGHGVCLKRELSLRPNNNQFTRLFFNPLPNHSVSLPARGFTPLAVFKSRTKAPPKKVEKPKQKVEDGIFGTSGGIGFTKQNELFVGRVAMIGFAASLLGEAITGKGILAQLNLETGIPIYEAEPLLLFFILFTLLGAIGALGDRGKFIDDPEPATGLERAVIPPGKSFRSALGLKEGGPLFGFTKANELFVGRLAQLGFAFSLIGEIITGKGALAQLNIETGVPINEIEPLVLLNVVFFFIAAVNPGTGKFVTDEEDDE

SEQ ID NO: 34 - protein, 272 aa, Cucumis sativus
MAAQTMLLTSGVCVGHGVCLKRELSLRPNYTQFTRLFFNPLPSHSVSLPPRGFTTLAVFKSRTKAPPKKVEKPKQKVEDGIFGTSGGIGFTKQNELFVGRVAMIGFAASILGEAITGKGILAQLNLETGIPIYEAEPLLLFFILFTLLGAIGALGDRGKFIDDPEPATGLERAVIPPGKSFRSALGLKEGGPLFGFTKANELFVGRLAQLGFAFSLIGEIITGKGALAQLNIETGVPINEIEPLVLLNVVFFFIAAVNPGTGKFVTDEEDDE

SEQ ID NO: 35 - protein, 267 aa, Eutrema salsugineum
MAQTMMLTSGVSANQFLRNKNPLAQPKVHHLFLSGNSPVVIPSRRQSLVPLALFKPKTKAAPKKVEKVKPKVEDGIFGTSGGIGFTKANELFVGRVAMIGFAASLLGEAVTGKGILAQLNLETGIPIYEAEPLLLFFILFTLLGAIGALGDRGKFVDDPPTPTGLEKAVIAPGKNVRSALGLKEQGPLFGFTKANELFVGRLAQLGIAFSLIGEIITGKGALAQLNIETGIPIQDIEPLVLLNVAFFFFAAINPGNGKFITDDGEDM

SEQ ID NO: 36 - protein, 265 aa, Arabidopsis lyrata
MAQTMLLTSGVSAGQFLRNKSPLAQPKVHHLFLSGNSPVVLPSRRQSFVPLALFKPKTKAAPKKVEKPKKKVEDGIFGTSGGIGFTKANELFVGRVAMIGFAASLLGEALTGKGILAQLNLETGIPIYEAEPLLLFFILFTLLGAIGALGDRGQFVDDPPTGLEKAVIPPGKNVRSALGLKEQGPLFGFTKANELFVGRLAQLGIAFSLIGEIITGKGALAQLNIETGIPIQDIEPLVLLNVAFFFFAAINPGNGKFITDDGEES

SEQ ID NO: 37 - protein, 265 aa, Camelina sativa
MAQTMLLTSGVSAGQFLRNKNPLAQPKVHHLFLSGTSPVALPSRRQSFVPLALFKPKTKAAPKKVVKPKDKVEDGIFGTSGGIGFTKANELFVGRVAMIGFAASLLGEALTGKGILAQLNLETGIPIYEAEPLLLFFILFTLLGAIGALGDRGKFVDDPPTGLEKAVIPPGKGVRSALGLKEQGPLFGFTKANELFVGRLAQLGIAFSLIGEIITGKGALAQLNIETGIPIQDIEPLVLLNVAFFFFAAINPGNGKFITDDGEES

SEQ ID NO: 38 - protein, 265 aa, Capsella rubella
MAQTMLLTSGVSAGQFLRNKNPLAQPKVHQLFLSGTSPVALPSRRQSFVPLALFKPKTKAAPKKVEKVKPKVEDGIFGTSGGIGFTKANELFVGRVAMIGFAASLLGEGITGKGILAQLNLETGIPIYEAEPLLLFFILFTLLGAIGALGDRGKFVDDPPTGLEKAVIPPGKGVRSALGLKEQGPLFGFTKANELFVGRLAQLGIAFSLIGEIITGKGALAQLNIETGIPIQDIEPLVLLNVAFFFFAAINPGNGKFITDDGEES

SEQ ID NO: 39 - protein, 265 aa, Noccaea caerulescens
MAQTMLLTSGVSANQFLRNKNPLAQPKIQHLFLSGNSPVALPSRRPSFVPLALFKPKTKAAPKKVEKAKPKVEDGIFGTSGGIGFTKQNELFVGRVAMIGFAASLLGEALTGKGILAQLNLETGIPIYEAEPLLLFFILFTLLGAIGALGDRGKFVDDPPTGLEKAVIPPGKSVRSALGLKEQGPLFGFTKSNELFVGRLAQLGIAFSLIGEIITGKGALAQLNIETGIPIQDIEPLVLLNVAFFFFAAINPGNGKFITDDGEES

SEQ ID NO: 40 - protein, 265 aa, Brassica napus
MAQTMLLTSGISANHFLRNKNPLAQPKVHHLFLSGNSPVTLPSRIPSLVPLAIFKPKTKAAPKKVEKVKPKVEDGIFGTSGGIGFTKQNELFVGRVAMIGFAASLLGEALTGKGILAQLNLETGIPIYEAEPLLLFFILFTLLGAIGALGDRGKFVDDPPTGLEKAVIPPGKGVRSALGLKEQGPLFGFTKANELFVGRLAQLGIAFSLIGEIITGKGALAQLNIETGIPIQDIEPLVLLNVAFFFAAAINPGNGKFITDDGEES

SEQ ID NO: 41 - protein, 265 aa, Raphanus sativus
MAQTMLLTSGISANHFLRNKNPLAQPKVHHLFLSGNSPVTLPSRRPSLVPLAIFKPKTKAAPKKVEKVKPKVEDGIFGTSGGIGFTKQNELFVGRVAMIGFAASLLGEALTGKGILAQLNLETGIPIYEAEPLLLFFILFTLLGAIGALGDRGKFVDDPPTGLEKAVIPPGKGVRSALGLKEQGPLFGFTKANELFVGRLAQLGIAFSLIGEIITGKGALAQLNIETGIPIQDIEPLVLLNVAFFFFAAINPGNGKFITDDGEES

SEQ ID NO: 42 - protein, 265 aa, Brassica rapa
MAQTMLLTSGISANHFLRNKNPLAQPKVHHLFLSGNSPVTLPSRRPSLVPLAIFKPKTKAAPKKVEKVKPKVEDGIFGTSGGIGFTKQNELFVGRVAMIGFAASLLGEALTGKGILAQLNLETGIPIYEAEPLLLFFILFTLLGAIGALGDRGKFVDDPPTGLEKAVIPPGKGVRSALGLKEQGPLFGFTKANELFVGRLAQLGIAFSLIGEIITGKGALAQLNIETGIPIQDIEPLVLLNVAFFFAAAINPGNGKFITDDGEES

SEQ ID NO: 43 - protein, 265 aa, Raphanus sativus
MAQTMLLTSGVSANTFLRNKNPLAQPKVHHLFLSGNSHVILPSRRPSLVPLAIFKPKTKAAPKKVEKVKPKVEDGIFGTSGGIGFTKQNELFVGRVAMIGFAASLLGEALTGKGILAQLNLETGIPIYEAEPLLLFFILFTLLGAIGALGDRGKFVDDPPTGLEKAVIPPGKGVRSALGLKEQGPLFGFTKANELFVGRLAQLGIAFSLIGEIITGKGALAQLNIETGIPIQDIEPLVLLNVAFFFFAAINPGNGKFITDDGEES

SEQ ID NO: 44 - protein, 265 aa, Brassica napus
MAQTMLLTSGVSANQFLRNKNPLAQPKVHHFFLSGNSHVVLPSRRPSLVPLAIFKPKTKAAPKKVEKVKPKVEDGIFGTSGGIGFTKQNELFVGRVAMIGFAASLLGEALTGKGILAQLNLETGIPIYEAEPLLLFFILFTLLGAIGALGDRGKFVDDPPTGLEKAVIPPGKGVRSALGLKEQGPLFGFTKANELFVGRLAQLGIAFSLIGEIITGKGALAQLNIETGIPIQDIEPLVLLNVAFFFFAAINPGNGKFITDDGEES

SEQ ID NO: 45 - protein, 265 aa, Brassica rapa
MAQTMLLTSGVSANQFLRNKNPLAQPKVHHFFLSGNSHVVLPSRRPSLVPLAIFKPKTKAAPKKVEKVKPKVEDGIFGTSGGIGFTKQNELFVGRVAMIGFAASLLGEALTGKGILAQLNLETGIPIYEAEPLLLFFILFTLLGAIGALGDRGKFVDDPPTGLEKAVIPPGKGVRSALGLKEQGPLFGFTKANELFVGRLAQLGIAFSLIGEIITGKGALAQLNIETGIPIQDIEPLVLLNVAFFFFAAINPGNGKFITDDGEER

SEQ ID NO: 46 - protein, 274 aa, Phoenix dactylifera
MVHAVFTSGLGAHLPDSKREPLLQSQLRRLRPTPFSHLLLSQPSHKQLPSPSYSSYTPVLALFKSKTKAPPKKVEKAKPKVEDGIFGTSGGIGFTKQNELFVGRVAMIGFAASLLGEAITGKGILAQLNLETGIPIYEAEPLLLFFILFTLLGAIGALGDRGRFVDDPPTGLDKAVIPPGKGIRSALGLKEGGPLFGFTKSNELFVGRLAQLGIAFSIIGEIVTGKGALAQLNIETGVPINEIEPLVLFNVIFFFIAALNPGNGKFVTDVDEEE

SEQ ID NO: 47 - protein, 270 aa, Anthurium amnicola
MAQSMLMSSLGSRLVETKGEPLLQFQIRRLRPTPATHLLLPPPTRRHPLPPSYAPTLAAFKSKTKAAPKKVEKGNFKVEDGIFGTSGGIGFTKQNELFVGRVAMIGFAASLLGEAITGKGILAQLNLETGIPIYEAEPLLLFFILFTLLGAIGALGDRGRFVDDPPTGLDKAVVPPGKGFRSSLGLKEGGPLFGFTKSNELFVGRLAQLGIAFSIIGEIITGKGALAQLNIETGVPISDIEPLVLLNVVFFFLAALNPGTGQFVTDDGED

SEQ ID NO: 48 - protein, 269 aa, Trifolium subterraneum
MAQTMLLMSSVSSTYSVELKKDPLLQLQCQRLRSRISDVSFNPLSSNSKSLSSRTFTTLALFKSKTKAPAKVVKPKPKVEDGIFGTSGGFGFTKQNELFVGRVAMIGFAASILGEALTGKGILAQLNLETGIPIYEAEPLLLFFIIFTLLGAIGALGDRGKFVDDEPTTGGVIPPGKGFRAALGLREGGPLFGFTKSNELFVGRLAQLGFVFSLIGEIITGKGALAQLNVETGVPITEIEPLVLFNVIFFFIAALNPGTGTFVTDEEED

SEQ ID NO: 49 - protein, 270 aa, Pisum sativum
MAQTMLLMSSVSSTYSVPLNKDPLLQLQCQRLKPRFSDISFSPLSSNSKSFSSRTFKTLALFKSKTKAPAKVVPKQKPKVEDGVFGTSGGFGFTKQNELFVGRVAMIGFAASILGEGITGKGILSQLNLETGIPIYEAEPLLLFFIIFTLLGAIGALGDRGKFVDDEPTTGGVIPPGKGFRQALGLSEGGPLFGFTKSNELFVGRLAQLGFAFSLIGEIITGKGALAQLNIETGVPINEIEPLVLFNVAFFFIAALNPGTGKFVTDEEED

SEQ ID NO: 50 - protein, 271 aa, Beta vulgaris
MAQAMLLMPSVSTTNTIDLKRNALLKIQIQKIKPKSSSSHLFFSPLSSSSSSSSTFKTLALFKPKTKAPVKKVEKPKLKVEDGLFGTSGGIGFTKENELFVGRVAMIGFAASLLGEGLTGKGILAQLNLETGIPIYEAEPLLLFFILFTLLGAIGALGDRGRFVDDPPTGLEKAVIPPGKNVRSALGLREGGPLFGFTKSNELFVGRLAQLGFAFSLIGEIITGKGALAQLNIETGVPVNEIEPLVLLNVLFFFIAAINPGTGKFITDEEE

SEQ ID NO: 51 - protein, 274 aa, Cephalotus follicularis
MAQTMLLMCGISTSHVVDLRRDPLFHVQIQKLRPKSFSHLFFNPLSNNGFSLAQKFNTLALFKSKTKAAPKKVATTAKPKVEDGLFGTSGGIGFTKQNELFVGRVAMIGFAASLLGEAVTGKGILSQLNLETGIPIYEAEPLLLFFILFTLLGAIGALGDRGRFVDDEPTGLERAVIPPGKGIRGALGLREGGPLFGFTKSNELFVGRLAQLGIAFSLIGEIITGKGALAQLNIETGIPINEIEPLVLFNVIFFFFAALNPGTGKFVTDEEAED

SEQ ID NO: 52 - protein, 665 aa, Tarenaya hassleriana
MGSTLFCSPVNPSLSKLDFSRTHLLSPADKHFFLDLPSFMGKVRGKSSVMSRRRVVGVKATAAVEEERRETAAEKEKLRVLVAGGGIGGLVFALAAKRKGFDVAVFEKDLSAIRGEGQYRGPIQIQSNALAALEAIDMGVAEEVMEAGCITGDRINGLVDGVSGSWYVKFDTFTPAAERGLPVTRVISRMTLQQILARAVGEDVIQNESNVVSFSESEDKVTVVLENGECYQGDLLVGADGIWSKVRSQLFGHEEATYSGYTCYTGIADFVPADIESVGYRVFLGHKQYFVSSDVGGGKMQWYAFHEEPAGGRDAPDGRKKRLLEIFQGWCDNVIDLLHATEEDAILRRDIYDRTPSFTWGKGRVTLLGDSIHAMQPNMGQGGCMAIEDSYQLAWELEHAWRRSVETDKPIDIVSSLRSYEEARKLRVAIIHGMARMAAIMASTYKGYLGVGLGPLSFLTMLRIPHPGTVGGRFFIDVAMPLMLNWVLGGNSSKLEGRLPCCRLSDKADDQLQRWFQDNDALERTVIGDWYLIPYGNDSIVSETVRLDKDEDQPCIIGSEPDEDFPGMRIEIPSSQVSKVHARIIYKDGAFFVMDLRSEYGTYITDNEGRRYRVTPNFPARFRPSDIIELGSDKKAAFRVKVIRKTPKMTRRDDRESSSKLLQAA

SEQ ID NO: 53 - protein, 663 aa, Arabis alpina
MGSTPFCYTINPSPSKLDFTKTHLAKQFYLDLSSFTGKSGAGLSGIKNRRGVVGVKASATALVEEEKRETVTEKKKKSRVLVAGGGIGGLVFALAAKKKGFDVLVFEKDLSAIRGEGQYRGPIQIQSNALAALEAIDIAVAEEVMEAGCITGDRINGLVDGVSGTWYVKFDTFTPAASRGLPVTRVISRMTLQQILARAVGEEVIRNESNVVDFEDSGDKVTVVLENGQRYEGDLLVGADGIWSKVRNNLFGRSEATYSGYTCYTGIADFIPADIESVGYRVFLGHKQYFVSSDVGGGKMQWYAFYEEPAGGVDAPNGMKKRLFDIFEGWCDNVLDLLHATEEEAILRRDIYDRSPSFTWGKGRVTLLGDSIHAMQPNMGQGGCMAIEDSYQLASELEDAWKQSVDTNRPVDVVSSLKRYEESRRLRVAIIHGMARMAAIMASTYKAYLGVGLGPLSFLTKFRVPHPGRVGGRFFIDIAMPLMLNWVLGGNSEKLEGRAPSCRLTDRADDRLREWFDDNEALERTISGEWYLIPQGDDCCVSETLCLTKDEDQPCIIGSEPDKDFPGMHIVIPSSQVSKMHARIIYKDGAFFVMDLRSEHGTYVVDNEGGRYRATPNFPARFRPSDILEFGSDKKAAFKVKVIRKTPKSTSKNESNGKLLQAA

SEQ ID NO: 54 - protein, 665 aa, Camelina sativa
MGSTPFCYSINPSPSKLDFTRTHVFSPVSKQFYLDLSSFTGKSAGGGGGFRSRRALVGVKAATALVEEKRESGTEKKKKSRVLVAGGGIGGLVFALAAKKKGFDVLVFEKDLSAIRGEGQYRGPIQIQSNALAALEAIDTDVAEQVMEAGCITGDRINGLVDGISGTWYVKFDTFTPAASRGLPVTRVISRMTLQQILARAVGEEVIRNESNVVDFEDSGDKVTVVLENGERYEGDLLVGADGIWSKVRNNLFGRSEASYSGYTCYTGIADFIPADIESVGYRVFLGNKQYFVSSDVGGGKMQWYAFHEEPAGGADAPNGMKKRLFEIFDGWCDNVLDLLHATEEDAILRRDIYDRNPSFTWGKGRVTLLGDSIHAMQPNMGQGGCMAIEDSFQLALELDEAWKQSVETKTPVDVVSSLKRYEESRRLRVALIHGMARMAAIMASTYKAYLGVGLGPLSFLTKFRVPHPGRVGGRFFIDIAMPMMLNWVLGGNSEKLEGRSPSCRLTDKADDRLREWFEDDDALERTIKGEWYLIPHGDDCCISETLCLTKDEDQPCIIGSEPDQDLPGKHIVIPSSQVSKMHARVIYKDGAFFLMDLRSEHGTYVTDNEGRRYRATPNSPARFRTSDIIEFGSDKKAAFRVKVIRTTPKSTRKNESNDKLLQAA

SEQ ID NO: 55 - protein, 668 aa, Camelina sativa
MGSTPFCYSINPTPSKLDFTRTHVFSPVSKQFYLDLSSFTGKSGVGAGGFRSLRALVGVKAATALVEEEQKRESVTDKTKKKSRVLVAGGGIGGLVFALAAKKKGFDVLVFEKDLSAIRGEGQYRGPIQIQSNALAALEAIDTDVAEQVMVAGCITGDRINGLVDGISGTWYVKFDTFTPAASRGLPVTRVISRMTLQQILARAVGEELIRNESNVVDFEDSGDKVTVVLENGERYEGDLLVGADGIWSKVRNNLFGRSEASYSGYTCYTGIADFIPADIESVGYRVFLGNKQYFVSSDVGGGKMQWYAFHEEPAGGADAPNGMKKRLFEIFDGWCDNVLDLLHATEEDAILRRDIYDRNPSFTWGKGRVTLLGDSIHAMQPNMGQGGCMAIEDSFQLALELDEAWKQSVKTKTPVDVVSSLKRYEESRRLRVALIHGMARMAAIMASTYKAYLGVGLGPLSFLTKFRVPHPGRVGGRFFIDIAMPMMLNWVLGGNSEKLEGRSPSCRLTDKADDRLREWFEDDDALERTIKGEWYLIPHGDECCISETLCLTKDEDQPCIIGSKPDQDLPGKHIVIPSSQVSKMHARVIYKDGAFFLMDLRSEHGTYVTDNEGRRYRATPNSPARFRTSDIIEFGSDKKAAFRVKVIRTTPKSTRKNESNDKLLQAA

SEQ ID NO: 56 - protein, 669 aa, Capsella rubella
MGSTPFCYSINPSPSKLDFTRTHVFSPVAKQFYLDLSSFTGKSGVGGGLSGFRSRRTLVGVKAATALVEEEQKPGEVTERKKKSRVLVAGGGIGGLVFALAAKKKGFDVLVFEKDLSAIRGEGQYRGPIQIQSNALAALEAIDNDVAEQVMEAGCITGDRINGLVDGVSGTWYVKFDTFTPAASRGLPVTRVISRMTLQQILARAVGEEVIRNESNVVDFEDSGDKVTVVLENGQRYEGDLLVGADGIWSKVRNNLFGRSEAEYSGYTCYTGIADFIPADIESVGYRVFLGHKQYFVSSDVGGGKMQWYAFHEEPAGGTDAPNGMKKRLFEIFDGWCDNVLDLLHATEEDAILRRDIYDRSPSFTWGKGRVTLLGDSIHAMQPNMGQGGCMAIEDSFQLALELEEAWKQSVETNTSVDVVSSLKKYEESRRLRVAIIHGMARMAAIMASTYKAYLGVGLGPLSFLTKFRVPHPGRVGGRFFIDIAMPLMLNWVLGGNSEKLEGRPPSCRLTDKADDRLREWFENDDALERTINGEWYLIPHGDECCVSETLCLTKDEDQPCIVGSEPDQDLPGKHIVIPSSQVSKMHARVIYKDGAFFLMDLGSEHGTFVTDNEGRRYRATPNFPGRFRSSDIIEFGSDKKAAFRVKVIRKTPKATRKNESNDKLLQAA

SEQ ID NO: 57 - protein, 667 aa, Arabidopsis lyrata
MGSTLFCYSINPSPSKLDFTRTHVVSPVAKQFYLDLSSFSGKSGGGLSGFRSRKTLVGVKAATALVEKEEKREAVTEKKKKSRVLVAGGGIGGLVFALAAKKKGFDVVVFEKDLSAIRGEGQYRGPIQIQSNALAALEAIDTDVAEQVMEAGCITGDRINGLVDGVSGSWYVKFDTFTPAASRGLPVTRVISRMTLQQILARAVGEDVIRNESNVVDFEDSGDKVTVVLENGQRYEGDLLVGADGIWSKVRNNLFGRSEATYSGYTCYTGIADFVPADIESVGYRVFLGHKQYFVSSDVGGGKMQWYAFHEEPAGGVDAPNGMKKRLFEIFDGWCDNVLDLLHATEEDAILRRDIYDRSPSFTWGKGRVTLLGDSIHAMQPNMGQGGCMAIEDSFQLALELEEAWKQSVGTNTPVDVVSSLKRYEESRRLRVAIIHGMARMAAIMASTYKAYLGVGLGPLSFLTKFRVPHPGRVGGRFFVDIAMPLMLDWVLGGNSEKLEGRPPSCRLTDKADDRLREWFEDDDALERTIKGEWYLIPHGEDCCVSETLCLTNDEDQPCIVGSEPDQDFPGMRIVIPSSQVSKMHARVIYKDGVFFLMDLRSEHGTYVTDNEGRRYRATPNFPARFRSSDIIEFGSDKKAAFRVKVIRKTPKSTRKNESNDKLLQTA

SEQ ID NO: 58 - protein, 662 aa, Raphanus sativus
MGSSTLFCYSINPSPSKLDFTKTHVFSPVAKQFYLDFPSFAAGKLRLRKRRALVGAKAETLLEEKRETVTEKKKKPRVLVAGGGIGGLVFALAAKKKGFDVLVFEKDLSAIRGEGQYRGPIQIQSNALAALEAIDIGVAEEVMEAGCITGDRINGLVDGVSGTWYVKFDTFTPAASRGLPVTRVISRMTLQQILARAVGEEIIRNESNVVDFQDSGDKVTVVLENGERYDGDLLVGADGIWSKVRNNLFGRSEATYSGYTCYTGIADFVPADIESVGYRVFLGHKQYFVSSDVGGGKMQWYAFHEEAAGGVDAPNGMKKRLFDIFEGWCDNVLDLLQATEEEAILRRDIYDKSPSFTWGKGRVTLLGDSIHAMQPNMGQGGCMAIEDSFQLGLELEAAWKQSVETNTPVDVVSSLRRYEESRRLRVAIIHGMARMAAIMASTYKAYLGVGLGPLSFLTKFRVPHPGRVGGRFFIDIAMPLMLNWVLGGNSEKLEGRPPSCRLTDKADDRLREWFEDDEALERTINGEWYLIPHGNECSVSETLRLTKDEEQPCIVGSEPDQDFPGTHIVIPSPQVSKMHARVIYKDGAFFLMDLRSEHGTYLTDNEGGKYRVTPNFPTRFRSSDIIEFGSDKKAAFRVKVIRTTPKLTRRDEKSDGKLLQAA

SEQ ID NO: 59 - protein, 687 aa, Brassica napus
MKCTSHIKLPLRASRIEMGSSTPFCYSINPSPSKLDFTRTHAFSPVAKQFYLDLPSFAGKSGGGLSGLRKRRTLIGVKAAAATLVAEEEKRETVTESKKKPRVLVAGGGIGGLVFALAAKKKGFDVLVFEKDLSAIRGEGQYRGPIQIQSNALAALEAIDIGVAEEVMEAGCITGDRINGLVDGVSGTWYVKFDTFTPAASRGLPVTRVISRMTLQQILARAVGEEIIRNESNVVDFEDSGDKVTVVLENGQRYDGDLLVGADGIWSKVRNNLFGRSEATYSGYTCYTGIADFVPADIESVGYRVFLGHKQYFVSSDVGGGKMQWYAFHEEAAGGVDAPNGMKKRLFDIFEGWCDNVLDLLQATEEEAILRRDIYDRSPSFTWGKGRVTLLGDSIHAMQPNMGQGGCMAIEDSFQLGLELEQAWKQSVETNTPVDVVSSLRRYEESRRLRVAIIHGMARMAAIMASTYKAYLGVGLGPLSFLTKFRVPHPGRVGGRFFIDIAMPLMLNWVLGGNSEKLEGRPPSCRLTDKADDRLREWFEDDEALERTINGEWYLIPHGNECSVSETLRLTKDEDQPCIVGSEPDQDFPGMHIVIPSPQVSKMHARVIYKDGAFFVMDLRSEHGTYLTDNEGGKYRVTPNFPARFRPSDIIEFGSDKKAAFRVKVIRTTPKLTRRDEKSDGKLLQAA

SEQ ID NO: 60 - protein, 668 aa, Brassica rapa
MGCSTPFCYSINPSPSKLDFTKTHAFSPVAKQFYLDLPSFAGKSGGGLSGLRKRRALVGVKAATLLAEEEKRETVTESKKKPRVLVAGGGIGGLVFALAAKKKGFDVLVFEKDLSAIRGEGQYRGPIQIQSNALAALEAIDIGVAEEVMEAGCITGDRINGLVDGVSGTWYVKFDTFTPAASRGLPVTRVISRMTLQQILARAVGEEIIRNESNVVDFEDSGDKVTVVLENGQRYDGDLLVGADGIWSKVRNNLFGRSEATYSGYTCYTGIADFVPADIESVGYRVFLGHKQYFVSSDVGGGKMQWYAFHEEAAGGVDAPNGMKKRLFDIFEGWCDNVLDLLQATEEEAILRRDIYDRSPSFTWGKGRVTLLGDSIHAMQPNMGQGGCMAIEDSFQLGLELEQAWKQSVETNTPVDVVSSLRRYEESRRLRVAIIHGMARMAAIMASTYKAYLGVGLGPLSFLTKFRVPHPGRVGGRFFIDIAMPLMLNWVLGGNSEKLEGRPPSCRLTDKADDRLREWFEDDEALERTINGEWYLIPHGNECSVSETLRLTKDEEQPCIVGSEPDQDFPGTHIVIPSPQVSKMHARVIYKDGAFFVMDLRSEHGTYLTDNEGGKYRVTPNFPARFRPSDIIEFGSDKKAAFRVKVIRTTPKLTRRDEKSDGKLLQAA

SEQ ID NO: 61 - protein, 668 aa, Brassica rapa
MGCSTPFCYSINPSPSKLDFTKTHAFSPVAKQFYLDLPSFAGKSGGGLSGLRKRRALVGVKAATLLAEEEKRETVTESKKKPRVLVAGGGIGGLVFALAAKKKGFDVLVFEKDLSAIRGEGQYRGPIQIQSNALAALEAIDIGVAEEVMEAGCITGDRINGLVDGVSGTWYVKFDTFTPAASRGLPVTRVISRMTLQQILARAVGEEIIRNESNVVDFEDSGDKVTVVLENGQRYDGDLLVGADGIWSKVRNNLFGRSEATYSGYTCYTGIADFVPADIESVGYRVFLGHKQYFVSSDVGGGKMQWYAFHEEAAGGVDAPNGMKKRLFDIFEGWCDNVLDLLQATEEEAILRRDIYDRSPSFTWGKGRVTLLGDSIHAMQPNMGQGGCMAIEDSFQLGLELEQAWKQSVETNTPVDVVSSLRRYEESRRLRVAIIHGMARMAAIMASTYKAYLGVGLGPLSFLTKFRVPHPGRVGGRFFIDIAMPLMLNWVLGSNSEKLEGRPPSCRLTDKADDRLREWFEDDEALERTINGEWYLIPHGNECSVSETLRLTKDEEQPCIVGSEPDQDFPGTHIVIPSPQVSKMHARVIYKDGAFFVMDLRSEHGTYLTDNEGGKYRVTPNFPARFRPSDIIEFGSDKKAAFRVKVIRTTPKLTRRDEKSDGKLLQAA

SEQ ID NO: 62 - protein, 667 aa, Noccaea caerulescens
MGSTPFCYSTNPSPSKLDFTRTHAFSPLAKQFYLDLSSLAGKSGGGLSGFRSPRTLVGVKAAAATALVEEEKREVVAEKKKSRVLVAGGGIGGLVFALAAKKKGFDVLVFEKDLSAIRGEGQYRGPIQIQSNALAALEAIDTDVAEEVMEAGCITGDRINGLVDGVSGTWYVKFDTFTPAASRGLPVTRVISRMTLQQILARAVGEEIIRNESNVVDFEDSGDKVTVVLENGERYEGDLLVGADGIWSKVRNNLFGRSEATYSGYTCYTGIADFVPADIESVGYRVFLGHKQYFVSSDVGGGKMQWYAFHEEPAGGVDPPNGMKKRLFDIFEGWCDNVLDLLHATEEDAILRRDIYDRTPSFTWGKGRVTLLGDSIHAMQPNMGQGGCMAIEDSYQLAMELEGAWKQSVETNTPVDVVSSLKKYEESRRLRVAIIHGMARMAAIMASTYKAYLGVGLGPLSFLTKFRVPHPGRVGGRFFIDIAMPLMLNWVLGGNSEKLEGRSPSCRLTDKADDRLREWFEDNDALERTINGEWYLIPHGDDSSVKETISLTKDEDQPCIIGSEPDQDLPGMHIVIPSSQVSKMHARVTYKDGAFFLMDLGSEHGTYVVDNEGRRYRASPNYPARFRSSDIIEFGSDKKAAFRVKVIRTTPKSTSKNESNGKLLQAA

SEQ ID NO: 63 - protein, 666 aa, Eutrema salsugineum
MGSTPFCYSINPSPSKLDFTRTHVFSPVAKQFYLDLSSCAGKSGGGLSGFRSRRALVGVRAATALVEEEKREIAKEKKKPRVLVAGGGIGGLVFALAAKKKGFDVLVFEKDLSAIRGEGQYRGPIQIQSNALAALEAIDIDVAEEVMEAGCITGDRINGLVDGVSGTWYVKFDTFTPAASRGLPVTRVISRMTLQQILARAVGEEVIRNESNVVDFEDSGDKVTVVLENGERYEGDLLVGADGIWSKVRNNLFGRSEATYSGYTCYTGIADFVPADIESVGYRVFLGHKQYFVSSDVGGGKMQWYAFHEEPAGGVDAPNGMKKRLFDIFEGWCDNVLDLLHATEEEAILRRDIYDRTPSFNWGKGRVTLLGDSIHAMQPNMGQGGCMAIEDSYQLALELEEAWERSVETNAPVDVVSSLRRYEESRRLRVAIIHGMARMAAIMASTYKAYLGVGLGPLSFLTKFRVPHPGRVGGRFFIDIAMPLMLNWVLGGNSEKLEGRPPSCRLTDKADDRLREWFEDDDALERTINGEWYLIPYGNECSVSETLCLTKDEDQPCIIGSEPDQDFPGMHIVIPAPQVSKMHARVTYKDGAFFLMDLRSEHGTYVTDNEGRRYRVTPNFPARFRSSDIIEFGSDKKAAFRVKVIRTTPKSTSKNKESNGKLLQAV

SEQ ID NO: 64 - protein, 666 aa, Eutrema halophilum
MGSTPFCYSINPSPSKLDFTRTHVFSPVAKQFYLDLSSCAGKSGGGLSGFRSRRALVGVRAATALVEEEKREIAKEKKKPRVLVAGGGIGGLVFALAAKKKGFDVLVFEKDLSAIRGEGQYRGPIQIQSNALAALEAIDIDVAEEVMEAGCITGDRINGLVDGVSGTWYVKFDTFTPAASRGLPVTRVISRMTLQQILARAVGEEVIRNESNVVDFEDSGDKVTVVLENGERYEGDLLVGADGIWSKVRNNLFGRSEATYSGYTCYTGIADFVPADIESVGYRVFLGHKQYFVSSDVGGGKMQWYAFHEEPAGGVDAPNGMKKRLFDIFEGWCDNVLDLLHATEEEAILRRDIYDRTPSFNWGKGRVTLLGDSIHAMQPNMGQGGCMAIEDSYQLALELEEAWERSVETNAPVDVVSSLRRYEESRRLRVAIIHGMARMAAIMASTYKAYLGVGLGPLSFLTKFRVPHPGRVGGRFFIDIAMPLMLNWVLGXNSEKLEGRPPSCRLTDKADDRLREWFEDDDALERTINGEWYLIPYGNECSVSETLCLTKDEDQPCIIGSEPDQDFPGMHIVIPAPQVSKMHARVTYKDGAFFLMDLRSEHGTYVTDNEGRRYRVTPNFPARFRSSDIIEFGSDKKAAFRVKVIRTTPKSTSKNKESNGKLLQAV

SEQ ID NO: 65 - protein, 665 aa, Cucumis sativus
MALTRFHNPFNLSSSSLSRTCFPVPAFREYLVEISPCQRIGCNFGGKSACGRRKKLTQVKAAVTEAPPAEGAAGEISRSLPTKNVRILVAGGGIGGLVFALAAKRKGFDVVVFEKDISAIRGEGQYRGPIQIQSNALAALEAIDLDVAEEVMRVGCITGDRINGLVDGVSGNWYIKFDTFTPAAERGLPVTRVISRMSLQQILARAVGDDVIINDSNVVDFEDSGEKVKVTLENGQQHEGDLLVGADGIWSKVRKNLFGHSEAVYSGYTCYTGIADFIPADIETVGYRVFLGHKQYFVSSDVGAGKMQWYAFHKEPPGGADAPNGKKERLFKIFEGWCDNVTDLIQATDEDSVLRRDIYDRTPIFTWGKGRVTLLGDSVHAMQPNMGQGGCMAIEDGYQLALELDKAWNESVASGSPIDIVSSLKSYESSRRIRVAVIHGMARMAALMASTYKAYLGVGLGPLSFLTQFRIPHPGTFGGRFFIDLAMPLMLNWVLGGNSSKLEGRPPACRLSDKANDQLRKWFEDDDALERAINGDWFLLPQGGEASVSQPICLRKDENQPCLIGSVEKEVDSGLSVAIPLPQVSEKHARIYYKDGAFFLTDLRSEHGTWLSDHEGRRYRAPPNFPVRFHQSDLIEFGSDKKARFRVKVIRSSVENDREKVEMNS

SEQ ID NO: 66 - protein, 658 aa, Vitis vinifera
MASAVFYSSVQPSIFSRTHIPIPISKDSFEEFGHSINYKHYFRSNPCGQKKRVAQVKATLAEATPAPPAPSLPSKKVRILVAGGGIGGLVLALAAKKKGFDVVVFEKDMSAIRGEGQYRGPIQIQSNALAALEAVDMEVAEEVMRAGCITGDRINGLVDGVSGDWYVKFDTFTPAAERGLPVTRVISRMTLQQILARAVGEDIIMNGSNVVDFEDDGNKVTVILENGQRYEGDLLIGADGIWSKVRKSLFGPKEATYSGYTCYTGIADFVPADIDSVGYRVFLGHKQYFVSSDVGAGKMQWYAFYNEPAGGVDGPEGKKERLLKIFGGWCDNVIDLILATDEEAILRRDIYDRTPTFTWGRGRVTLLGDSVHAMQPNMGQGGCMAIEDSYQLAMELDKAWEQSIKSGTPIDVVSCLKSYEKARRIRVAVIHGMARMAAIMASTYKAYLGVGLGPLSFLTKLRIPHPGRVGGRFFIDIAMPLMLSWVLGGNSSKLEGRPPSCRLSDKANDQLRRWFEDDDALERAIGGEWFLLPSGESGLQPICLSKDENKPCIIGSVSHTDFPGISTVIPSPKVSKMHARISCKDGAFFLTDLQSEHGTWITDNVGRRQRVSPNFPTRFHPSEVIDFGSEKASFRVKVVRTPPDNAAKNEESKLFQAV

SEQ ID NO: 67 - protein, 664 aa, Corchorus olitorius
MATTLFQNPSTFFTGTQFPVSIPKYVPTESSACLHCNYHFRGKASKQKKRFLQVKATVAGTQSDSKSDEKNKVDANQQEKKKARILVAGGGIGGLVFALAAKNKGFDVVVFEKDLSAIRGEGQYRGPIQIQSNALAALEAIDMEVAEKVMEAGCITGDRINGLVDGVSGSWYIKFDTFTPAAERGLPVTRVISRMTLQQILARAVGEDIIFNESNVVDFEDDGNKVSVVLENGKRFEGDLLVGADGIWSKVRKNLFGPKDAVYSGYTCYTGIADFVPADIDSVGYRVFLGHKQYFVSSDVGGGKMQWYAFHKEPAGGVDTQGKKERLLKIFEGWCDNVIDLLLATDEEAILRRDIYDRTPSLTWGKGRVTLLGDSIHAMQPNLGQGGCMAIEDGYQLALELDKAWKQSVESGTPIDVVSSLRSYESARRLRVAIIHGMARMAAIMASTYKAYLGVGLGPLSFLTKYRIPHPGRVGGRFFIDIAMPLMLSWVLGGNSSKLEGRSLTCRLSDKASDQLREWFEDNDALERAISGEWFLFPAGDGVSSQPICLSRDENNPFMIGSEKKEDFPGTSVVIPSSQVSKTHAQITYKDGAFFVTDLQSEHGTYIINQEGKKSRVTPNVPTRIRPSDVIEFGSDKKAAFRVKAIKSAPKIAEKEGSGVLQAA

SEQ ID NO: 68 - protein, 661 aa, Populus euphratica
MASSTLFGNTLTAVSSRTHFPTPIFNNSLELLSSTHSNYNFKTKTSTSAKKLKVEAVVTETPAVSKSEGKQSEQRKLKVLVAGGGIGGLVFALAAKNKGFDVMVFEKDLSAVRGEGQYRGPIQIQSNALAALEAIDLDVAEEVMRAGCITGDRINGLVDGISGTWYVKFDTFTPAAERGLPVTRVISRMTLQQILARSVGDDTILNDSNVVSFQDDGDKVTVVLENGQQYEGDLLVGADGIWSKVRKNLFGPKEAVYSGYTCYTGIADFVPVDIETVGYRVFLGHKQYFVSSDVGAGKMQWYAFHKESPGGMDAPHGKKDRLLKIFEGWCDNVIDLLLTTDEDSILRRDIYDREPIITWGKGRVTLLGDSVHAMQPNMGQGGCMAIEDSYQLASELERAWKQSIESGTPVDVLSSLRSYENARRLRVAIIHGMARMAAIMASTYKAYLGVGLGPLSFLTNFRIPHPGRVGGRFFIDIAMPVMLDWVLGGNSSKLEGRSLSCRLSDKASDQLRRWFVDDDALERSLNGEWFLLPCGNDAVASQPIGLSRDENKPCVVGSVSQEDFPGMSIVIPAPQVSKTHARITCKDGAFYLIDLRSEHGTFITDIEGRRYRAPPNFPTRFHPSDMIEFGSDKKVIFRVKVMRSPPKISEKKDEGQVLQSV

SEQ ID NO: 69 - protein, 665 aa, Ricinus communis
MASSAFFCNSINPSTSVFSRTHFSFPIFSTSTVEFSSFAQYNFHFKTKKSDHQNKRFTQVKAVVTESPTVAESNGKLSEQKKLRILVAGGGIGGLVFALAAKRKGFEVLVFEKDLSAIRGEGQYRGPIQVQSNALAALEAIDLEVAEEVMRAGCITGDRINGLVDGVSGTWYCKFDTFTPAAERGLPVTRVISRMTLQQILACAVGEDVIMNASNVINFQDNEDKVTVTLENGQQFEGDLLVGADGIWSKVRKNLFGPKEATYSGYTCYTGIADFVPVDIESVGYRVFLGHKQYFVSSDVGAGKMQWYAFHNEPPGGVDSPNGKKERLLKIFEGWCDNVIDLLHATDEDAILRRDIYDREPVFTWGKGRVTLLGDSIHAMQPNMGQGGCMAIEDSYQLALELDKAWKQSIESGTPVDVVSSLKSYERTRRLRVAIIHGMARMAAIMASTYKAYLGVGLGPLSFLTKYRIPHPGRVGGRFFIDIAMPVMLNWVLGGNSSKLEGRPLSCRLSDKASDQLQTWFEDDNALERALNGEWFLLPFGDDAVQEPICLSRDENIPCMVGSESQEDFPGKSIVISSPQVSKMHARISYKDGGFYVIDLQSEHGTFITDNDGRRSRVPPNFPTLFHPSEAIEFGSAGKAKFRVKVMKSPAKIKEKGGNEILQSV

SEQ ID NO: 70 - protein, 665 aa, Jatropha curcas
MASSTLFCNLINPSISVFSRTHLPFPIVSSSSMELSSSAHCNFHFRSKKSEQNKKLTQVKAVVTESTSVAQSDEKQPEQRKPRILVAGGGIGGLVFALAAKRKGFEVLVFEKDLSAVRGEGQYRGPIQIQSNALAALEAIDLEVAEEVMEAGCITGDRINGLVDGVSGTWYVKFDTFTPAAERGLPVTRVISRMTLQQILALAVGEDVIRNESNVVSFQDDGDKVTVTLENGQHFEGDLLVGADGIWSKVRKNLFGPKDATYSDYTCYTGIADFVPADIESVGYRVFLGHKQYFVSSDVGAGKMQWYAFHKEPPGGVDRPNGKKERLLKIFEGWCDNVIDLLLATDEDAILRRDIYDRTPTLTWGRGRVTLLGDSVHAMQPNMGQGGCMAIEDGYQLALEIEKAWKQSIESGTPIDIVSSLKSYERARRLRVAIIHGMARMAAIMASTYKAYLGVGLGPLSFLTKYRIPHPGRVGGRFFIDIAMPVMLSWVLGGNSSKLEGRSQCCRLSDKASDQLRTWFEDDDALERALNAEWFLLPLGNKVVVSEPICLSRNENTPCVVGKVSHKDFPGMSIVIPSPQVSEMHARISYKDGAFYVIDLQSEHGTYISDADGRRYRVPQNSPTRFHPSDVIEFGSDEKAKFRVKVMKSPSRIKEKEGSEILQSV

SEQ ID NO: 71 - protein, 480 aa, Eucalyptus grandis
MASMAAGSICLFRDESIGAASIAVTILANRSSHMAKLLCYRGKARATMWMGIKTTRRLAMKCSLESHCRSESSCSGFPLPRKEEVTSSRGSRSGVPKVTESAREIFHLVREWSQRRVIQLAALAACTFLIIPSADAVDALKTCACLLKECRIELAKCVANPSCAANVACLQTCNNRPDETECQIKCGDLFENSVVDEFNECAVSRKKCVPRKSDVGEFPVPDPAVLVKSFNMADFTGKWFISSGLNPTFDTFDCQLHEFHTESNKLMGNLTWRIRTPDSGFFTRTAVQRFVQDPEQPGILYNHDNEYLHYQDDWYILSSKTENKPDDYVFVYYRGRNDAWDGYGGAVVYTRSAVLPDSIVPELERAAKSVGRDFKDFIRTDNTCGPEPPLVERIEKTVEEGEKTIVREVEQIEDEVQKVGKTEMTLFQRLTEGFRELRQDEENFLRRLSQEEMEVLNELKMEATEVEKLFGRALPIRKLR

SEQ ID NO: 72 - protein, 458 aa, Tarenaya hassleriana
MAVAVQSFSSPCYRDTQFISGNRSNRKLLRKRMVDSDTIVLVKICSGARGKIHCSLKARFSSPSSGYKSGLSKGRPPFEALTLWNKLKEQSRQSLLKLVGILACTFLIVPSVDAVDALKTCACLLKECRIELAKCISNPSCAANVACLQTCNNRPDETECQIKCGDLFENSVVDEFNECAVSRKKCVPRKSDLGEFPAPDPAVLVKRFNIGDFSGKWYISSGLNPTFDTFDCQLHEFHTESDKLVGNLSWRIRTPDGGFFTRSTVQKFVQDPNQPGILYNHDNEYLHYQDDWYILSSKVENGPDDYVFVYYRGRNDAWDGYGGAVVYTRSAVLPNNIVPELERAAKSVGRDFSKFIRTDNTCGPEPPLVERIEKTVEEGERTIIREVEQIEGEVAKVGETEMTLLQRLVEGFKELKQDEENFLQGLNKEEMELLSELKMEASEVEKLFGKALPIRKLR

SEQ ID NO: 73 - protein, 461 aa, Camelina sativa
MAVATHCFTSPCHDRIRFFSSDDGIGRLGISRKRINGTFLVKILPPIQNVDLRTNGRSSRPLSAFRSGISKGVFDILSLPSKDELKELSTPLLLKLVGVFACAFLIVPSAEAVDALKTCACLLKGCRIELAKCISNPACAANIACLQTCNNRPDETECQIKCGDIFENKVVDEFNECAVTRKKCVPKKSDLGEFPAPDPSVLVQNFNIGDFSGKWYITSGLNPTFDAFDCQLHEFHTEGENKLVGNISWRIKTLDSGFFTRSAVQTFVQDPNQPGVLYNHDNEYLHYQDDWYILSSKIENKPEDYIFVYYRGRNDAWDGYGGAVVYTRSSVLPNSIVPELEKAAKSIGRDFSTFIRTDNTCGPEPPLVERLEKTVEEGERIIVKDFEEIEEEVEKEVEKVGKTEMTLFQRLAEGFNELRKDEENFVRELSKEEMEFLDEIKMEANEVEKLFGKALPIRKVR

SEQ ID NO: 74 - protein, 461 aa, Capsella rubella
MAVATHCFTSPCHDRIRFFSSDDGIARLGISRKRINGTFLVKILSPIQSADLRTSGRSSRPLSAFRSGFSKRVFDIVSLPSKNELKELSTPLLLKLVGVLACAFLIVPSAEAVDALKTCACLLEGCRIELAKCIANPACAANVACLQTCNNRPDETECQIKCGDLFENSVVDEFNECAVSRKKCVPRKSDLGEFPAPDPSVLVQNFNIGDFNGKWYITSGLNPTFDAFDCQLHEFHTEGDNKLVGNISWRIKTLDSGFFTRSAVQTFVQDPNQPGVLYNHDNAYLHYQDDWYILSSKIENKPEDYIFVYYRGRNDAWDGYGGAVVYTRSSVLPNSIVPELEKAAKSIGRDFSTFIKTDNTCGPEPPLVERLEKTVEEGERIIVKEVEEIEEEVEKEVEKVGKTEMTLFQRLAEGFNELKQDEENFVRELSKEEMEFLDEIKMEASEVEKLFGKALPIRKVR

SEQ ID NO: 75 - protein, 455 aa, Arabidopsis lyrata
MAVATHCFTSPCHGRIRFFSSDDGRLGITRKRINGTFLLKILPPIQNAGGRSSRPISAFRSGFSKGIFDIVALPSKNELKELTTPLLLKLVGVLACAFLIVPSADAVDALKTCACLLKGCRIELAKCIANPACAANVACLQTCNNRPDETECQIKCGDLFENSVVDEFNECAVSRKKCVPRKSDLGEFPAPDPSVLVQNFNISDFNGKWYITSGLNPTFDAFDCQVHEFHTEGDNKLVGNISWRIKTLDSGFFTRSAVQKFVQDPNQPGVLYNHDNEYLHYQDDWYILSSKIENKPDDYIFVYYRGRNDAWDGYGGAVVYTRSSVLPNSIIPELEKAAKSIGRDFSTFIRTDNTCGPEPPLVERIEKTVEEGERIIVKEVEEIEEEVEKEVEKVGKTEMTLFQRLAEGFNELKQDEENFVREFSKEEMEFLDEIKMEASEIEKLFGKALPIRKVR

SEQ ID NO: 76 - protein, 457 aa, Brassica napus
MSVSTHCFTSPCHDRTRFFSGDDGNKLLRKRIKGTFLVKILPSSQNAYLRITAKSSRPLSGFRSGISKGVFDIVALTSKNALKELTTPLMLKLVGVVACAFLIVPSADAVDALKTCACLLKGCRIELAKCIANPSCAANVACLQTCNNRPDETECQIKCGDLFENSVVDEFNECAVSRKKCVPRKSDLGEFPAPDPSVLVKNFNINDFDGKWYITSGLNPTFDAFDCQLHEFHTEDGKLVGNISWRIKTPDSGFFTRSTVQKFVQDPNQPAVFYNHDNEYLHYQDDWYILSSKIENKPEDYIFVYYRGRNDAWDGYGGAVVYTRSASLPNTIVPELEKAAKSIGREFSTFIKTDNTCGPEPPLVERLEKTVEEGEKIIVKEVEEIEEEVEKEVEKVGKTEMTLFQRLAEGFEELKQDEENFLRGLSKEEMELLDELKMEANEVEKLFGKALPIRKFR

SEQ ID NO: 77 - protein, 491 aa, Brassica rapa
MSYPKGISSHPYISLFLFSLYLPPLLPAELSVSSMSVSTHCFTSPCHDRTRFFSGDDGNKLLRKRIKGTFLVKILPSSQNAYLRVTAKSSRPLSGFRSGISKGVFDIVALTSKNALKELSTPLMLKLVGVVACAFLIVPSADAVDALKTCACLLKGCRIELAKCIANPSCAANVACLQTCNNRPDETECQIKCGDLFENSVVDEFNECAVSRKKCVPRKSDLGEFPAPDPSVLVKNFNVKDFDGKWYITSGLNPTFDAFDCQLHEFHTEDGKLVGNISWRIKTPDSGFFTRSTVQKFVQDPNQPAVFYNHDNEYLHYQDDWYILSSKIENKPEDYIFVYYRGRNDAWDGYGGAVVYTRSASLPNTIVPELEKAAKSIGREFSTFIKTDNTCGPEPPLVERLEKTVEEGEKIIVKEVEEIEEEVEKEVEKVGKTEMTLFQRLAEGFEELKQDEENFLRGLSKEEMELLDELKMEANEVEKLFGKALPIRKFR

SEQ ID NO: 78 - protein, 493 aa, Noccaea caerulescens
LSLSLSLSLSLSLLPAELSIFGIASGRLATESMSVATHCFTSPTCHDRIRFFSRITSDDGRLVRKRINGTFLLKILPPSQNAHLRTTLRSSRPLSAGFRSGISKGVIDIVALTSRNALKELSTPLLLKLVGVLACAFLIVPSADAVDALKTCACLLKECRIELAKCIANPSCAANVACLQTCNNRPDETECQIKCGDLFANSVVDEFNECAVSRKKCVPRKSDLGEFPVPDPSALVKNFNIQDFNGKWYITSGLNPTFDIFDCQLHEFHVQGNKLVGNISWRIKTPDTGFFTRSAVQTFKQDPNQPAVLYNHDNEYLHYQDDWYILSSKIENKPEDYIFVYYRGRNDAWDGYGGAVVYTRSNVLPNSIVPELEKAAKSIGRDFNTFVRTDNTCGPEPPLVERLEKTVEEGEKMIEKEVEEIEEEVEKEVEKVGKTEMTLFQRLAEGFNELKQDEENFLRGLSEEEMALLDELKMEAGEVEKLFGKALPIRKFR

SEQ ID NO: 79 - protein, 459 aa, Eutrema salsugineum
MAVATYCFTSPCHDRIRFFSGVTSFDGRLLRKRINGTFLVKILPPSQNADLRTTSRSSRPLSGFRSGTSKGVFDIVALTSRNALKELSTPMVLKLVGVLACAFLIVPSADAVDALKTCACLLKGCRIELAKCIANPSCAANVACLQTCNNRPDETECQIKCGDLFENKVVDEFNECAVSRKKCVPQKSDLGEFPVPDPSVLVKNFNISDFNGKWYITSGLNPTFDTFDCQRHEFHADGDKLVGNISWRIKTLDSGFFTRSAVQKFVQDPNQPAILYNHDNEYLHYQDDWYILSSKVENKPDDYIFVYYRGRNDAWDGYGGAVVYTRSAVLPNSIVPELEKAAKSIGRDFSTFVRTDNTCGPEPPLVERFEKTVEEGEKIIVKEVEEIEEEVEKEVEKVGKTEMTLFQRLAEGFNELKQDEENFLRGLSEEEMELLNELKMEASEVEKLFGKSLPIRKVR

SEQ ID NO: 80 - protein, 481 aa, Prunus humilis
MALAGRSIFLSHNDSIGNTCIRLGITSDEGFQKRRMVGFHVVVKFPSNRRKSRYSQFIRSKRNFCVLGSKCSSLLSSKTEEAFSKCSTRTSEPEMKRVISFLLEEASSCIKEWSQLHFMKVAGLLACTFMIMPSANAADALKTCACLLKECRVELAKCIGNPSCAANIACLQTCNNRPDETECQIKCGDLFENSVVDEFNECAVSRKKCVPKKSDVGEFPIPDPAVLVKSFDIEKFNGKWFITSGLNPTFDAFDCQLHEFHTESSKLVGNLSWRIRTPDGGFFTRSAVQKFVQDPNQPGILYNHDNDYLHYQDDWYILSSKMENKPDDYIFVYYRGRNDAWDGYGGAVIYTRSSVLPASIVPDLEKAAASVGRDFSKFIRTDNTCGPEPPLVERLEKTLEEGERNIIEEVKQIEGEVEKVEQTELTLLQKLAEGFNELKQDEENFLRGLSKEEMDILSELKMEAGEVEKLFGQTLPLRKLR

SEQ ID NO: 81 - protein, 481 aa, Pyrus x bretschneideri
MAFAGRSVFLSHKESIGNACIRLGLKSDERLQKGRMVGFHVVVKFSSQRRKSRYCQFISAKRNFSGLGSKRSSLLSSRTEEAFSERSTKTSEPEMGRAIDLMIEEVLSVIKEWSQLHFMKVAGLLLCTFMVMPSANAVDALKTCACLLKECRVELAKCIGNPSCAANIACLQTCNNRPDETECQIKCGDLFENKVVDEFNECAVSRKKCVPMKSDVGEFPIPDPAALVKSFDMSKFNGKWFITSGLNPTFDVFDCQLHEFHTESSKLVGNLSWRIKTPDGGFFTRSAVQKFVQDPNQPGILYNHDNEYLHYQDDWYILSSKIENTPEDYIFVYYRGRNDAWDGYGGAVVYTRSSVLPESIVPELERAAKSVGRDFSKFIRTDNTCGPEPPLVERLEKTIEEGERTIIEEVKQIEGEVEKVEQTELTLFQKLLEGFNVLKQDEENFLRGLSKEEMDLLSELKMEAGEVGKLFGKTLPLRKLR

SEQ ID NO: 82 - protein, 471 aa, Bixa orellana
MALAPHSTHFSELTGRRFLSRKVVHLHGMVLLRIQSSDRKSRYSKSINPYRNHIVSKLRCSDQLSEGTHNISSTCSSNTRRKHEAKEGFEFVVPNLPNILRKWSQLQIMKVAGVLACALLVIPSASAVDALKTCTCLLKECRIELAKCIANPSCAANVACLQTCNNRPDETECQIKCGDLFENSVVDVFNECAVSRKKCVPQKSDVGEFAVPDPSVLVKNFNIADFSGKWYISSGLNPSFDTFDCQLHEFHTESNKLVGNLSWRIRTPDGGFFTRSTLQKFVQDPKQPGILYNHDNEYLHYQDDWYILSSKIENKQDDYVFVYYRGRNDAWDGYGGAVVYTRSAVLPESIVPELERAAKCVGRDFNKFIKTDNTCGPEPPLVERLEKKVEEGEETIIREVEQLEGQVEKVGKTEMTLFQKLAEGFKELQKDEENFLEELSKEEMDVLSELKMEAREVEKLFGGALPLRKLR

SEQ ID NO: 83 - protein, 487 aa, Theobroma cacao
MAQAARSLCFSHDKSVQVPCRTSGLTSNERFHRRQIAHFHGIMLVKIQSSGRKARYSQLNKSNPNYSASDLRCSNQLSRRKDRNFSSCSCNRRRPKAEEAFAFLVPTISNVLKEWSQSKIVKVVGLLACAYLVIPSASAVDALKTCTCLLKECRIELAKCIANPSCAANIACLQTCNDRPDETECQIKCGDLFENSVVDEFNECAVSRKKCVPQKSDIGEFPVPSPAVLVKNFNIADFSGKWFISSGLNPTFDTFDCQLHEFHTEAGKLVGNLSWRIGTPDGGFFTRSTLQRFVQDPNYPGILYNHDNEYLHYQDDWYIISSKIENKQDDYIFVYYQGRNDAWDGYGGAVVYTRSAVLPESIVPELEKAAKNVGRDFNKFIRTDNTCGPEPPLVERLEKKVEEGEQTLIREVKEIEGEVEKEVKRVEKTEMTLFQKLAEGFKELQQDEENFLRGLSKEEMGLLNELKMEASEVEKLFGEALPLRKLR

SEQ ID NO: 84 - protein, 479 aa, Gossypium arboreum
MAEAARLICFSHGKSVKVPCRTSGFTRKERFHRRLVAHFHGMMLLKLQSSCRNSRYSQLIKPNMNYSASKLRCSHQLSRRKERKFSSSSSNERTPKIPTISNLLEQWSQSQTVKLVGLLACAYLVIPSAAAVDALKTCTCLLKECRIELAKCIANPSCAANVACLQTCNDRPDETECQIKCGDLFENSVVDEFNECAVSRKKCVPQKSDIGEFPVPDPAVLVENFNIADFTGKWFISSGLNPTFDTFDCQLHEFHIEGGKLVGNLSWRIRTPDGGFFTRSATQRFVQDPNHPGILYNHDNEYLHYQDDWYIISSKIENKPDDYVFVYYRGRNDAWDGYGGAVVYTRSAVLPETIVPELKRAAQNVGRNFNKFIRTDNSCGPEPPLVERLEKKVEEGEQTLIREVEQIEGEVEKGVEKVEKTEQTLFQRLAEGFKELQQDEENFLRGLSKEEMELLNDLKMEASEVEKLFGEALPIRKLR

SEQ ID NO: 85 - protein, 487 aa, Gossypium hirsutum
MAEAARLICFSHGKSVKVPCRTSGFTRKERFHRRLVAHFHGMMLLKLQSSCRNSRYSQLIKPNMNYSASKLRCSHQFSRRKERKFSSSSSNERTPKAEEVFSFQIPTISNLLEQWSQSQTVKLVGLLACAYLVIPSAAAVDALKTCTCLLKECRIELAKCIANPSCAANVACLQTCNDRPDETECQIKCGDLFENSVVDEFNECAVSRKKCVPQKSDIGEFPVPDPAVLVENFNIADFTGKWFISSGLNPTFDTFDCQLHEFHIEGGKLVGNLSWRIRTPDGGFFTRSATQRFLQDPNHPGILYNHDNEYLHYQDDWYIISSKIENKPDDYVFVYYRGRNDAWDGYGGAVVYTRSAVLPETIVPELKRAAQNVGRNFNKFIRTDNSCGPEPPLVERLEKKVEEGEQTLIREVEQIEGEVEKGVEKVEKTEQTLFQRLAEGFKELQQDEENFLRGLSKEEMELLNDLKMEASEVEKLFGEALPIRKLR

SEQ ID NO: 86 - protein, 489 aa, Populus trichocarpa
MALAANPFCLSQEQYIISSSSVAKSGLASDGRFHRRQRLHFHGAVLSQFWPNSRKLRYIQSIRSHRHHYGGVIRCSNQLSDWTKKFSSLCSSSSRSMPKAIERLNFVVLSVTNALKERNNLECLKLAGILLCALLVIPSADAVDALKTCTCLLKECRLELAKCIANPSCAANVACLQTCNNRPDETECQASSIKCGDLFENSVVDEFNECAVSRKKCVPRKSDVGEFPVPDPAVLVENFNMADFSGKWFITSGLNPTFDTFDCQLHEFHTESNKLVGNLSWRIKTPDTGFFTRSAVQRFVQDPVHPGILYNHDNEYLHYQDDWYILSSKIENKQDDYVFVYYQGRNDAWDGYGGAVVYTRSAVLPESIVPELEKAAKSVGRDFSKFIRTDNTCGPEPPLVERLEKTVEEGEKTIIKEVEEIEQKVEKAGKTELSLFQRLTEGFKEIQKDEENFLRELSKEETDLLNDLRMEAGEVEKLFGRALPIRKLR

SEQ ID NO: 87 - protein, 483 aa, Manihot esculenta
MAISANSVCLSHEESICSSYFRPGIASYERIHGRRGLNYQSIVVLNFWPNSRKSRYVQLMRTHKNYHGIKLRCSHGFSGWTKKFSSFCRTGANVTKAKEVLNFLMFSVSNNLKEKSHLQFLKVASILACVLLFIPSADAVDALKTCTCLLKECRLELAKCIANPACAANIACLQTCNNRPDETECQIKCGDLFENTVVDEFNECAVSRKKCVPKKSDVGEFPVPDPAVLVKSFNIADFNGKWYITRGLNPTFDTFDCQLHEFHVESNKLVGNISWRIRTPDSGFFTRSTVQRFVQDPMQPGILYNHDNEYLHYQDDWYILSSKVENKSDDYIFVYYRGRNDAWDGYGGAVVYTRSSVLPESIAPELERAAKSVGRDFSKFIRTDNTCGPEPPLVERLEKTVEEGEKSIIREVQEIEGEVEKVGKTEMTLFQRLAEGFKELQQDEEILLRKLSKEEMELFNDLKMEASEVEKLFGGALPLRKLR

SEQ ID NO: 88 - protein, 481 aa, Jatropha curcas
MALAANSIYLSHEESISSSYIKSGIERFHGRKGLHFRSIVVLKILPNSRKSRRHVQLIRSYRNYCGLVLRCSHQFSGWTNKFPSFSSTAASINKAKEVLNFLMLSVSNNLKEKGQLQFLKVAGILACALLVIPSADAVDALKTCACLLKECRLELAKCIANPACAANIACLQTCNNRPDETECQIKCGDLFENNVVDEFNECAVSRKKCVPKKSDVGEFPVPDPAVLVKSFNIADFSGKWFITSGLNPTFDTFDCQLHDFHRESNRLVGNLSWRIRTPDGGFFTRSTVQKFVQDPLQPGILYNHDNEYLHYQDDWYILSSKLENSENDYIFVYYRGRNDAWDGYGGAVVYTRSAVLPESIIPELETAAKKVGRDFNKFIITDNTCGPEPPLAERIEKTVEEGEKTIIREVEEIEGNVEKVGKTEMALFQRLAEGFKELQQDEEFFVRELSKEEMDILNDLKMEAGEVEKLFGEALPLRKLR

SEQ ID NO: 89 - DNA, 96 bp, synthetic construct
  source 1..96: mol_type = other DNA
gtaattaattaacgtttgaggttttagagctagaaatagcaagttaaaataaggctagtccgttatcaacttgaaaaagtggcaccgagtcggtgc

SEQ ID NO: 90 - DNA, 96 bp, synthetic construct
  source 1..96: mol_type = other DNA
aacaaggggaacagaattgggttttagagctagaaatagcaagttaaaataaggctagtccgttatcaacttgaaaaagtggcaccgagtcggtgc

SEQ ID NO: 91 - DNA, 96 bp, synthetic construct
  source 1..96: mol_type = other DNA
caaacagacccgtcactcacgttttagagctagaaatagcaagttaaaataaggctagtccgttatcaacttgaaaaagtggcaccgagtcggtgc

SEQ ID NO: 92 - DNA, 96 bp, synthetic construct
  source 1..96: mol_type = other DNA
tcttatctcatgttatgcgagttttagagctagaaatagcaagttaaaataaggctagtccgttatcaacttgaaaaagtggcaccgagtcggtgc

SEQ ID NO: 93 - DNA, 96 bp, synthetic construct
  source 1..96: mol_type = other DNA
ataacatctttaagttaacagttttagagctagaaatagcaagttaaaataaggctagtccgttatcaacttgaaaaagtggcaccgagtcggtgc

SEQ ID NO: 94 - DNA, 96 bp, synthetic construct
  source 1..96: mol_type = other DNA
attcccattctatcacatttgttttagagctagaaatagcaagttaaaataaggctagtccgttatcaacttgaaaaagtggcaccgagtcggtgc

SEQ ID NO: 95 - DNA, 96 bp, synthetic construct
  source 1..96: mol_type = other DNA
agttaaagccaacaaaaatcgttttagagctagaaatagcaagttaaaataaggctagtccgttatcaacttgaaaaagtggcaccgagtcggtgc

SEQ ID NO: 96 - DNA, 96 bp, synthetic construct
  source 1..96: mol_type = other DNA
gcgtagaagaggccaagtgcgttttagagctagaaatagcaagttaaaataaggctagtccgttatcaacttgaaaaagtggcaccgagtcggtgc

SEQ ID NO: 97 - DNA, 1811 bp, synthetic construct
  source 1..1811: mol_type = other DNA
gacgtggtcactctagttgcctgtcatcaactattacaccacatccacacccttgttaacttaaagatgttattcccattctatcacattttggtaatctcttaacatatagaacactaccctgaacatagtaaagaagccagtgctaggatttgttagtgaagcaatggctcaaaccatgttgctcatgtctagtgtctctagcagttattccgtggatttgaagaaagaccttttcctccaattgcagagtcaaagtttaaggcctaagttctctcagctctcattcaacccacttccatcatcaacttcttccttttcttcaccccgtacattcacaactctggccctcttcaaatctaagaccaaagccgctcctgctaagactaaggtactctttttccatattttataaaaggaaacttgtgtctaatctaataatgcttcatttttcagcacactgaatttggtttcacgatatattgcaggttacaaagccaaagcaaaaggttgaagatggtatctttggcacttctggaggatttggttttactaagcagaacgagctctttgtgggtcgtgttgctatgcttggttttgcagtgagtaaatcttagcttataaactccttaattatcacatcacaggagaattgagcgtatttgattagttcggaagatttataaaaatatactcttttaaatcaagttgattttgaatttgcaccacaagcattaacaaaatgttgcattaatttgcaggcatcactgttgggtgaaggaataactgggaaaggaattctagcacaattgaatctggaaactggaattcccctttatgaagcggagcctcttcttctgtttttcatcctcttcaccctgctaggagccattggaggtttaggtgaccgtggaaaatttgttgatgacgaacctactactggaggtgttattcctccaggcaaaggcttcagggaagctcttggtcttggttcaggtacttaaatttttaatttcttaactttataaaagagtagtgattagtagttaagtctaaatgaatgtgtttatatacaattatcaatatgtaattgtaatactaatagctaaatgtgtctcacaaattattaataatatcataaaaatcttattgaaatgtctaagtagcataaagttgctttttaatactatatatacatcatcatagtctaaattggttacatttttcataatataaaacttgggttttggcatttttcaggtcctttatttggattcacgaaagcaaacgagctatttgtgggaagattggctcaattgggtttcgttttctcattgattggagaaattataaccggaaagggagcactagcccaactcaacattgagactggggtaccaatcaacgaaatcgagcccctagtgttgttcaatgttcttttcttcttcattgctgctttgaatcctggaactggcaaattcgttacagatgagggggaggatgattagacactaacccttttgtttgtttgtgtttaaacaaatactcgtttgctcatctatcataaaatgcattgagttcatcataaacaacaaatctatatgatttttctccatagttgcacgcgtcatgattagacattattttttgctttggtaaattaaaatcatagtgcttcgatttgccagtaaaaaatcataatggaactcaaattagaacgcccatgttctatataaaatctaatggggtaaatgaaatgagatatcattccgcgtgattgaagaaagtgagaagatattagtattgggatc

SEQ ID NO: 98 - DNA, 12925 bp, synthetic construct
  source 1..12925: mol_type = other DNA
acccaacactagcaaccactgagttcacccctcaaacgttaattaattacaaggcttatgtctattttcagtatttttgacatcttcgttctcctgatttttgttggctttaactttaaaccaccaattctgttccccttgtttcttcatcattagattttaagtgccatgcattgatataatataaatatataacttcatttaattaatgcaagctattgagttttgagtcgaatctattagccacaaaacacacacacacatattctcacacaaactgcaaccatggctactaccttatgttacaattctcttaacccttcaacaaccgttttctcaagaacccatttctcagttcccttgaataaagagcttccactggatgcttcaccttttgttgttggctataactgtggtgtaggatgcagaacaaggaagcaaaggaagaaagtgatgcatgtgaagtgtgcagtggtggaggctccaccaggtgtttcaccctcagcaaaagatgggaatggaaccaccccttcgaagaagcagcttcgtatacttgtggctggtggagggattggagggttggtttttgctttggctgcaaagagaaaggggtttgaggtgatggtgtttgagaaggacttgagtgctataagaggggagggacagtataggggtccaattcagattcagagcaatgctttggctgctttggaagctattgattcagaggttgctgatgaagttatgagagttggttgcatcactggtgatagaatcaatggacttgtagatggggtttctggttcttggtaagtttaaaggttaagtacatatttgaattgaatcagtgatttgattgtcttttttctttttctttttttctttcttgttctatttttaggcatgcaatagggatactcatgctgattggtttaatatatcatttaaggtgttggaccctcaaaagctaattaggtagtttcaaccttgtggtaagtggcaactgtactcatttttggcaaatttaggacagagaaattctagcaatgagaaaagtacatggttaagggaaaagagtatattaaattattaatatcaattgagaactttgagttgacaaacttcaatatcatgaccgttgcaagaagtgccctagtaaaaaaactgtgaaatcttaacaacagcagcagggagtcaaacgcaactgcaatggaaattcagatgtttaacaatgtttgtgtcagtggtcctagttaacaagtctattaacatcctgtttgttaatgagtttgattactcttagtgagtacatccttcattttaaagttgggggaacttgggcttaataggattctgtagaactaaactaaccaaataattattctagcaaacatgcaaatgggtgttctgttattgtaaattttcctttctttcacaaacaacaatcttatttccttactatcaagttataaaaaaaaatgttttcttctattttctgtatgaaattttagcatttggtaccgtgaattgatatataggttgttgtcaggtacgtcaagtttgatacattcactcctgcagtggaacgtgggcttcctgtcacaagagttattagtcgaatggttttacaagagatccttgctcgcgcagttggggaagatatcattatgaatgccagtaatgttgttaattttgtggatgatggaaacaaggtaattagagaaagattgttaaagattctcctcatatttctgtctattatgatatctattttctgatgcttttatttctttcttattgaaatttgaaaacgcattggcagacaaatatcatttgattgatgtttactactttcccaccctgcattccacaatttcaggtaacagtagagctagagaatggtcagaaatatgaaggagatgtcttggttggagcggatggaatatggtccaaggtcttattaatttttctctgattatgatcttattctttagcaaataatttttcccatatgaaagtaaacatcatactgaaacatgacaagtactgtggagctcatatgtgtgttagatttgttctttcattgtgaagtagtgaactctttctgtcattctgtctgtaagcggttcgtataatcataaattctgcatttaattttattgtaattttttatgaataggtgaggaagcagttatttgggctcacagaagctgtttactctggttatacttgttatactggcattgcagattttgtgcctgctgacattgaaactgttgggtatgttgattatattcatgtatcgtatctctatttatccattttctgtggactatgttgaaattttgtaaacattacttacattgagttgagaagcttacttgaacttcttcgctggaagataccgagtattcttgggacacaaacaatactttgtatcttcagatgttggtgcgggaaagatgcaatggtatgcatttcacaaagaacctcctggtggtgttgatgagcccaacggtatgcattctctttgtgtttgagatttgaaaacagttagaagcacaccctttgcaaatgttaaattttcaatgtggtctacttatagacatacctttttaattgtttgtgttggcttttacataactgatgccgttttttaattgattgtacctggaaagaattttcaactgttatggtaatgaataattgattatttgtttgacattttgagcttagatcatttctatgctgaggatcactgattattatgttctgccactagttactactaataatgaacaattagtgggggttatatatgaaaaacgaaaaaaaaaggtaaagaaagctgctcatggcatcatctatttgtcggaatagaaggtattttattcaccttgttattcttgagtcatttacacagtataaatagatacaagagagagagagagagagtttaggggttgtcctagcctatacatgatactgatggctaaatataactaaatttgacccttgatctaagggatttgacagctatttacaattaatgttcttattttctggtaattctaatgactcgcgctaacactctccctcaagttagtacataaatgtctttaatgtccaacttgtcaagggagtcagggaagttcttatcggacactacctttgtaagaatgtctgtcaattggtcttccgacttgacaaacggaaatcaaataatcttcttgtcaagattttgcttgatgaaatgtctatctacctccaggtgctttgtttgatcatgctggatagaattttgtgagatatcaattgtatctttattgtcagagaacaagttcatctccatgctaggaccaaagccaatctccattagtaaccttttgagccataatagttcacaaagtcctttggtcatgcccctaaacttagcttcagcactagacaaggccactactttttgctttttacttctttgtgtaactaaattcccttccacaaatgtgaagtatcttgatgtggattttctgtctataacattccttgcccaatttgcatcagtgtacccttcaacactcaagtgattattcttagagaacatcaacccctttcttggtgaggattttaaatagcgaagtatttagtttattgcatctgtatgttcttcacttggattatgcataaattggctcaccacacttacggcataggcaatatcaaggtgagtatgtgacaaatagataagtttccccaccaatttttggtatcttcctttattcatgtgcacttggtctgtgtattgagaaagtttgtgattttggattgttggagtatctgttggtttgcagtccagcagccccaccttagtcaataagtcaaacacctactttctttgatacaagaaaatgcctttttgtgaccttgccacctttttccccaaaaaaatacttgaagtccacccaaattcttcatctcaaactcagttgtcaattgctcttgtagtcttgaaatttcttcgatatcatctccaataataatcatgtcatctacgtaaataattacagttgtcacatttccttgcagatgctttaagaatagggtgtgatttgaattactttgatcaaaatcataccttctcatagttgagctaaatcttccaaaccatgatcgaggttattctttcaatccatatagtgcttgctctaacttgcataccaccttggttccagaagagggcatatagctcgatgggatatctatataaacatcttcttcaagatctccatggagaaaagcgttcttcacgtcaacctgaagtaatgaccaatctaaatttgttgcaagagataataggactctaacaatatttagcttggccaacggtgaaaaagtctctgtataatctataccttaagtctatgtgaacccttttgccactagccttgccttgtgccgatcaatggatccatcacttttgtatttgattgagaacacccatttgcaccccaatatcttctttaccttaggaagtggtatcaaccttcatgaattattgttttgtaaagcttccatttcctcttgtatggctagtgcccacttaggatcagctaaggtttcttccacattttttggaatatgacaggataataatgtctttatgaaggcatgaataggatttgataaatgttgagtagacacataatttgcaatggggtatttggatcgcctttcttctacattagggcagtatctattgggtggctttcctcgattgttcctgataggcaatacatatcttacagaagcattagtagatgaggagatagtaggagagcttacctcaggattgttccaagtgagagggtcgggtattatgaaaagagggggctcttgacttccttgatcgatatagtcttcattgacgggaacttcttcactagcagttccttcattagtagaaggatcctctttacactcaacttcagttatatttaaatgttgcatttctataggttcatcatggattactccttcaacttcagattcatgctctccaatttgtttcttttcaatagctggtgatggagccaccaagtaccaatttagctctctattcatggactccccttgaagagctgaattggataccataggagaaaagaaaggttcatattctagctaggaaagttacatccatggtgatataagttttttcgatggtagggtccaagcatttgtagcattttttgtgtagtccatagcccaaaaacaagcacctaacaacacatgcatcaagtttggtgcgttggtttttgtggagatgaacaaaaaccacacacacaaaaatcctaggttaaaacatcaacacagtaggtaattgtacatgagaagagagtacttgcagaggtgttttaaaatgcaagaccttggagggcatgcagttgaggaggtatacaactgcaattacagcatcatcccaatggtgagttggaacatgggtgcaaagaagaaaagcacgtgttgtctctaagatatgacgatttttttgttcggcgacactattttgttgtggagtttgtgagcatgaggtctcctgaaagagaccatgttgttgggattatgctttgaaatgatggttaacaaactccattatatgaccgaagaacttgaattttggttgaaaactgattttgaatcatgatatgaaaattctgaaatatttgaaacacctcatctttagtcttcattaagtaaagccatgtcattcgtgtgcaatcatcgacaaatattacgaaccaacggtgaccaaatacgatagtcctaagggaaggtccccacacatcaaaatgttttagagcaaaaggaacactgcttttattcaaactcatacggtaggaggttctatggcttttagccaaaatacaagtcacacatgtaaacctaacatttttcaaatgtaaaaataaatatggaaataagtgtcttaaataactgaatgatggatgtcccaaatgacgatgccacaaccatatttctcattctttgctactagattgatggcacacgtgatgtgcttctcctttgctaaaattgtctaggtagtatagcccccctctcttagtaccacgcccaataatcttcttgttgagaacatcctgaagaaggaaaaaaagtagagtacatcaataccacataattgagctcttcagttatttgactcatagacattaatttattagataatgtaggaacgaggaaagtgtgagataaagagactgagggtgataatgccacagttccaacacctacgattggctatgcaactctatttgcattcgcaatgcaagtcctatgtggtggagttgactgggagaagttcatgggatcaaaggtcatatggtttgttgccccagagtcaataatccatgcacttgcatcctgagagttagaactacagaaaacttgacgccagttacctggtttcattgaggtggaggaactagttacaggacaaagcgagagttgagactcaacagtgactacaacagctttaccagtgccttcctcaagtgtcgtggtgtcttttttcttcttagcttgcaactcatgccactattttggatacccatgtagcttgaaacaggtatcacgattgtgtctaacatttccacaacgtgtgcactttcctccatcagatggacccttcgacttcaagactgagttgtgtttcccagccactagtgttggcaattggccaaattttgaacctttagtggccataatagcatcacttgttgtattatctaccccagatgccattatagtctaacaagtgtcctcccttcgaacatgtgcataagcatgctcgattgtgggaaatggcttcagttggagtatgtcactccaaatgttatcgagttggtcatccaatccattgagaaagatataaactctttcctcttgtagtaaataattgtatttttgtatatctcttgggcactccatggcattagggcaccgaaagtctatctcacgccacagcccttgtagaacattgtaatatttttcaattaatccacttttctacgacatctgggttaccttacgtcgaaggttcctaaagttgtgaagtatcagatccatcaaaatatgtcatagcaatggagtcccaaacccctttggctgttgggaagcggatgaaattccccaccagtgagggatccattgagttgatcaaccatcctttcactatagcgttctcagttcgccatttacagaatgtcgaatcaatttccgacgacaaagggaatgatccgtcaatatatcacagtttgtctttgcctgagatgtacatctcagccacctgggaccatagggcatagttggaaccatccaacttgatcctgatcgatatcgtcgaggaattagagattgacagttgtgatgttgactaggcttttgacaaaacctccatcatttttgtcgtaagatttgccaaaatggaatcggtagcggaagcatttgatggctactttgtggaatcaccaatcgctgaggaatcaaccatctttgggtcccaataatatgctcttgataccatgtcagaacataaggtattttattcaccttgttattcctttgagtcatttacagagtataaatagatacaagagggagagagcttaggagttgtcctagcctatacacgatactcatggctaaatataagtaaatttgacccttgatctaagagatttgacagctatttacaattaatgttcttattttctggtaattctaatgactcacgctaacattttttagcatttcaggaagcatgcttcatacctacaaaatactgatacttgcatttgtttcatgtatcgatattagacatgtatctgatattgatacttttggatacttcaccaataataagtatcagaaaactatcaagtgttagatttaactaactacatcaaatctcaattttaaagtgggtcacatttttactagttttaggaagaaatgaagtagctactattcttgtttgtaggaaataggctatgatttatgatgactatgattcatagtttctattttgtttttagtaatgttcaaaaagtgtgcttttagtttgaatttgtagagtaacaatgttatattatgtttaatattttatgcacacatgtatgtgtatcttctacattacgtatctgacattaagaataattgtatcatatccaatatgtatccatgcatcatagtagttagcatggtatttatttgagattacctcatgattgctttttgttaatattcaaagatggacctaaatgcttctctcgcccattctatctcagactcatttccttctattactgaatctttatccatctttcttgacgtggaatagaatggattgtaaatatcaagcattcaagctggtataatgatgaagattgctaatgaaatagcctgggttctatgacagtaatttagcattggaaatgtagaaataagatgaccttgaaaatatacaaatagatgtgtatctttatttttcatccttttcaatgaggaaaaaaaagttaaattatcatattcatatctaatttcttagttctttctgcatggcaggaaaaaaggaaaggttgcttaggatatttgagggctggtgtgataatgctgtagatctgatacttgccacagaagaagaagcaattctaagacgagacatatatgacaggataccaacattgacatggggaaagggtcgcgtgactttgcttggtgattccgtccatgccatgcagccaaatatgggccaaggagggtgcatggctattgaggtatatattctttttatctggtatttagcttattcatcctctctcaatgaaatatgaagttattatgagttctacattagtttcagaaattttttacttactcagaccaggaatggatgcaagaattgactaatgatgggggtggagggggaatacacaagataaacgaaaattcttagtttaaacatatgtatcatacatgttatttcatataataaatttcatctatgattgagttagtgtcaaatttttaaacaaagcaatgaaagactatttgtaccatatgactattatatcctccaagtcttggattgtttacatactgtttactcatccaaaattacaataagaatccacttttcattgatgtgaatctatttaagcttcctctttaaatattctctttttacctgtgggtcttcttacttataaaataaaaataaaaaattgttataaattataatgtattagcccattaaacacaaacttatcaaaactgattgtagaggctatttttgttttgcattaaaatttgattacctgtaatataaaatggaaatgtaataaggtcaatagggagactcctcggctctctctacctttgtccctctttcatacctaagacattatcactgattttctggactttggtatttgaaactttttcttaactttcttgtcgttaagctttctattattttttttcttggcacagctttcaaataaacaattttatttccatctgtctgatggatatacactatttacagaaacatacataaaattaaaatatttcctactttaagaggagatggagataatagatgtcaaactatctcaacttttgttagaggctcgaaagggactggtaatcaatgaaacacttgaaaagtgaggcaaaaactccttctgactagtttgttttatcatttcaggacagttatcaacttgcatgggagttggagaatgcatgggaacaaagtattaaatcagggagtccaattgacattgattcttccctaaggaggtaatataccattttgcatttatgcaaatgcaaatgatgatattattgacttgattcgaaaagagagatcgtttcttgcatcaaccatggctcaatagttaatgtttcattattggtttaaatacatttttgtttcttggaatttagtgatcttttgtttttcgttcttgtaaaaaaaaaaaaaatttcagtcattgtaaattatattatttttttgtttttagtctgtaaaatattttagatgacgttttgaacaataaaaaatgttatataaagtattataaggacgaaaaacaaatataatttgtaatgactaaaaaataattttttacaagaataaaaaaaaacgctaaattatcatgactgaaaagatatttaagcattcatcattttcaaattggtaaatttgatgataaatctctttggtgattaatggtgcccatcttttttatcccttttgcattaatactttcagctacgagagagaaagaagactacgagttgccattattcatggaatggctagaatggcggctctcatggcttccacttacaaggcatatctgggtgttggtcttggccctttagaagtaagaacttaaaatttatcaataagagaatgaaaaatatggtgaaatgacactcaaattgaaataattaaatgcacaaattcattcttgcttcccttactctgcagtttttgactaagtttcgtataccacatcctggaagagttggaggaaggttttttgttgacatcatgatgccttctatgttgagctgggtcttaggtggcaataggtaacgtttagagcctatttttctttgctcatagtctttctttcatattgtcttcatctgatcttcttattcacatgcatgtagaaatctgagccactggataaatgatgagaactaagaagttcaagaaattgataattgaaaacaatgtttagtctttacttggacaaacaataaagaaatatttatgtgtgcaattttgttactaacaacatgaaaaatgatgctacttttgcagcgacaaacttgagggcagaccactaagttgcaggctcacagacaaagtacgttgctcaaataaagtttcttttgtactttgttgctgagaagatattgtatatctggtaatttatatgttcacatctattagacttgttgtgatgttgttgttaatagctatttgattatttatgaaggcaaatgatcagttacgtagatggtttgaagatgatgaagcactcgagcgtgctattaatggagagtatgtctctgaccaaatatttgtatcacctattacatatttcttaccacaaataagtagatcacataacttaatgttcatccttgttttctcaattatagatggattttattaccgcatggagatggaacaggtctttcaaagcctatatctttaagtcgaaatgagatgaaacccttcataatcgggtatgcctattgctacacctacttacaatgcagtggcttaatagttcttaattatcataaagataatctaatttttggtcaccaattagtgttgagaagtgtgcagttcaattcctaattatcataaagataatctaattttcactttcagcctattttctaatatgtttttgtaattgtaggagtgcaccaatgcaagataattcaggcagttcagttacaatttcttcaccacaggtagcacaaatttatcattacctgaatgaaacacggtcccatacttgatgtgcttcacatcattaacataatcaacaatttctctttttaaggtttctccaacgcatgctcgaattaactataaggatggtgccttcttcttgattgatttacggagtgagcatggcacctggatcattgagtaagtaaaagaagtgaaaggttctttgtttgacattaaaagttttaaattcttgagaaatctatttaatatcattctcattatgttttcagcaacgaaggaaagcagtaccgggtacctcctaattatcctgctcgcatccgtccatctgatgttattcagtttggttctgagaaggtgagatgagaggtcctaaaaagcagttgcagtatactcatgttacacatttgcatcttctaggacttatattaggtatatgaggtaaccactgatcaacatatgagaaagatactacaactgtttgattcactaattatgactagtttctttcacagcctataagaattgaacattattagttggatgattcagtctcattgagtagtgttacttgtacacactttcatcccacactcaattgtttaaagtaaaattattagtttattgcattgtttaaccaataccgaaatttcagttgttaattttatttaaatacttgtgggagaggtgctaaaaaattattcaagttacatttgatttcacaattcccctcatttatctgagacaaatgattcacagacataatcttttgcgtatattatctgttatccttgcctgaaaatgttcaattttagtttaaatggtgcactacttatttgatgtctcagcaaccaagttatgtgcaggtttcgttccgtgttaaggtgacaagctctgttccaagagtctcagaaaatgaaagcacactagctttgcagggagtatgactgattctgctcaattgcaatttgtaagttatggaaaaattatacagcacaaatttgctattgtatagtactatctgcattgttttagggtggggtattataccacagtctagtcatttaagatctgatatgttacatgcctatatggacatttaagagggactcttgggtataaatttgttactccactccaatactttttgtgtatgacatttgtaatttgttagagttagatttataacatgacacacataaacttgcacgtgattaatgttgaatggagtaagcaatgcctagctatgaatttttgcacttttaattgtgatttacagaatcaaactgtatagtttatcattataagaagagcaaattcaattgtcaccaaatt

SEQ ID NO: 99 - DNA, 4017 bp, synthetic construct
  source 1..4017: mol_type = other DNA
ccttattggactttagaatattgagtccatttgcaaggaaattggaagcaaatagaggcgtcgacgtgtctgttgaggttccagtgagtgacgggtctgtttgcagtccacacaaacaccatcatcatcatgttcttatctcatgttatgcgacggcgactcttctttcttcttcttcctcttcttcctcacattttcttattctatttctcattttacctgcacttggcctcttctacgccgcaaggcatgtttccaactttgctcattctaaattatatctctgcccttaactactattataaactaaattttgtaacttggctaatgaaaacaagtgggtatgcaatattcttggacaattatattttccttttattatagagtctttagtattccattttgggctatgattacctctgcattctaaatgatatcctgttgatcttgttgatttggttttcacagtatcaggttacatttggagttgaagacatatacatatattaatatattattattattattatattatatgtatatatggcaacgtggggtgcgcactcaatgttgttatcccatggggaaggcactggcactcccaaagtatgtgtaaaagcaggccttacggttagaggtgatttaaggtttcacaggactagaggttttaaatatgctcctagtgtggtagtgctcaaagtgttggccaccgccagaaagccaacacggttgcgcttgttaagaccttattgcagtgtgagggggcaacacgaccatgggaaaggatatttttctcccactaattcacaaataccacgggttagtaaccgctgcttagtttatcctacttcattgttaatgcaatcatgcttatcccttttgtgaaaatgcaaaatttgttcctaagattgcacctttatatccaatgtccatcctctgagtttactttacaccttgtttggatagcaacatatattgaaattcgccgcggatcgaatgcttgcgtttctcaaagattggagtaatttacgcatcacggcagtagctggcatattggtatccgttttaatgatcattccaccagctgatgctgttgacgctctcaaaacttgtgcttgcttgttgaaggattgcaggtatgccatctttctttctcttgttcttgttctgccatgactacatgttatggccaccaatttcatactcttttgtctatgcaggatagaactggctaagtgtctttcaaacccatcttgtgcggccaatattgcttgtcttcaaacttgcaacaatagacctgatgagaccgaatgccaagttagtctcgcgctttacatcaaattcatgcctgcaccattattcatgtctccaaatcctgctcatgaagcttttattttcttcttatttttccagattaaatgtggggacctgtttgaaaacagtgtggttgatgaattcaatgaatgtgcagtatcacggaagaaatgtgtacctaagaaatctgatgtgggagaatttcctgctccaaatcctgatgtccttgttaatagctttaacattgctgagttcagtggcaagtggttcatcactagtggcttaaatcctaccttcgatacctttgactgccaattacatgaatttcacacagaatccaacaaacttgtgggtaatttgtcatggagaatacggactccagatgctggatttattactaggtccgctgagcagagatttgtacaagatccttcctatcctgggatcctttacaatcatgacaataagtaccttcactatcaagatgactggtaatttgttattcataagctttcttttcaatttttacttagctagacatgtatagttaattgcaactacatccacacgaagcacattgacgtgagggttgtgctagagccaaaataaaactagcttcctacttacttgtagagttgtagtgtaattaaggcacagaccaaatcatgtttcttaattaagttaggagagttagaaaaggttgttttgatcattatcaggagataaaaaaattcagcaaacttgaataaaaatttaattggtatgaagctcgacttaagaaattcacacacatggtagaattcatcacaaatcactatactgattctagatccaatcattgttaatggttcaattgtctactggaaaacatcctctgattcaatcaatgaaggacaacgccaatttgcaattgcaacaccaatgtttaggtccaattgatttaagaatgatccaatttaattagaccgcaaattcattagtgtagcttctaaacaaattctgaaataaattccaaaacctaatcagatctagttcaggcataaacatgcttataaacagatttggaaaagaaggggaagtttgaatccaaaatagaattaagaacaagtctcagaatccaaatcattagccttctatagcaatcaccaatggagaaactaaaaatgcataaaacgcaaacaaggggggaaaaccttgaatctgaaaggtcacaactacacaaatggtccaagtctatttttgcactatgtttttcctcttttgtaactaattatctcctaatcccaagattacccttatcccaactattagcccatgtaataagcttttcaacgatatctcatttacctaacttgcccttgaatttaatgatatatgctcaaaagacaaaaggtatgtcatgcatgctcgcccaatacttctggtctaactctaaagctgaattttaacactgaaattcccgaattcgtcctttttacaaagtttgtagatatctttattaactttcgtagcatataaagaacgtcataattagaggtctatagcttaagttatgactaaaatgctgatatagtgtcaaaatataactaatgctgtcaaaattattcactgcccaatttggcccaataattcaaattagtggtaggactaatcaatcaagaccaaaacctcataagcaaggcaattaaatatgttaaaaatagtagttatcatacaccctgtgtttgaaggaagtaaatgttcctttcctagatcagaaaaagaatacaacatgttgttccttttacgtgtgcatacattaaatcatttaacatttgccagatatttgaatcaaagagtcttttgttaacattagaagacttacaaacaatgatatatatatatatataggtatattttgtcatccaagattgagaataaatcagatgactacgtatttgtatactaccgaggcagaaacgatgcttgggatggttatggaggtgctgttgtatacacaaggagtgcagttttgcctgaatctatagttcctgaacttgaaaaagcatctaagagtgtgggaagagactttagcaagttcatcaggacagataacacatgcgggccagagccttccctggtggaaaggctggagaaaaaggtagaggaaggagaagagaccattgtaagggaagttgaacagatagaagaagaggtggagaaggtggggaaaacggaggcgaccttatttcagaagttggcagaagggttcaaaatgtttcaagaagacgaggaaaatttcttaagagggttatcaaaagaagaaatggagattcttgatgggcttaaaatggaagccggtgaagtagaaaagctctttggacgtgccttgccgattaggaaactaagataagtctgagaaccatgtccaagataacttggcatcataatctttttttatcatctttgcttttattcgttgactacatatattgcatggctgtagatagctttcctccttaatcattgcttttatgttgacatactaatgattattcaaatatttgtaacagcatttatcaattgttgtatatatattcagagtattgcatttgcataggtttagatgcttgtgctttttttcaagtcgaaaatatttggaatgggaattccgacatgaggaatgctgtatactaccagtgttcttcctcaactactaataca

SEQ ID NO: 100 - DNA, 1779 bp, synthetic construct
  source 1..1779: mol_type = other DNA
gacgtggtcactctagttgcctgtcatcaactattacaccacatccacacccttgtttttggtaatctcttaacatatagaacactaccctgaacatagtaaagaagccagtgctaggatttgttagtgaagcaatggctcaaaccatgttgctcatgtctagtgtctctagcagttattccgtggatttgaagaaagaccttttcctccaattgcagagtcaaagtttaaggcctaagttctctcagctctcattcaacccacttccatcatcaacttcttccttttcttcaccccgtacattcacaactctggccctcttcaaatctaagaccaaagccgctcctgctaagactaaggtactctttttccatattttataaaaggaaacttgtgtctaatctaataatgcttcatttttcagcacactgaatttggtttcacgatatattgcaggttacaaagccaaagcaaaaggttgaagatggtatctttggcacttctggaggatttggttttactaagcagaacgagctctttgtgggtcgtgttgctatgcttggttttgcagtgagtaaatcttagcttataaactccttaattatcacatcacaggagaattgagcgtatttgattagttcggaagatttataaaaatatactcttttaaatcaagttgattttgaatttgcaccacaagcattaacaaaatgttgcattaatttgcaggcatcactgttgggtgaaggaataactgggaaaggaattctagcacaattgaatctggaaactggaattcccctttatgaagcggagcctcttcttctgtttttcatcctcttcaccctgctaggagccattggaggtttaggtgaccgtggaaaatttgttgatgacgaacctactactggaggtgttattcctccaggcaaaggcttcagggaagctcttggtcttggttcaggtacttaaatttttaatttcttaactttataaaagagtagtgattagtagttaagtctaaatgaatgtgtttatatacaattatcaatatgtaattgtaatactaatagctaaatgtgtctcacaaattattaataatatcataaaaatcttattgaaatgtctaagtagcataaagttgctttttaatactatatatacatcatcatagtctaaattggttacatttttcataatataaaacttgggttttggcatttttcaggtcctttatttggattcacgaaagcaaacgagctatttgtgggaagattggctcaattgggtttcgttttctcattgattggagaaattataaccggaaagggagcactagcccaactcaacattgagactggggtaccaatcaacgaaatcgagcccctagtgttgttcaatgttcttttcttcttcattgctgctttgaatcctggaactggcaaattcgttacagatgagggggaggatgattagacactaacccttttgtttgtttgtgtttaaacaaatactcgtttgctcatctatcataaaatgcattgagttcatcataaacaacaaatctatatgatttttctccatagttgcacgcgtcatgattagacattattttttgctttggtaaattaaaatcatagtgcttcgatttgccagtaaaaaatcataatggaactcaaattagaacgcccatgttctatataaaatctaatggggtaaatgaaatgagatatcattccgcgtgattgaagaaagtgagaagatattagtattgggatc

SEQ ID NO: 101 - DNA, 12832 bp, synthetic construct
  source 1..12832: mol_type = other DNA
acccaacactagcaaccactgagttcacccctcattctgttccccttgtttcttcatcattagattttaagtgccatgcattgatataatataaatatataacttcatttaattaatgcaagctattgagttttgagtcgaatctattagccacaaaacacacacacacatattctcacacaaactgcaaccatggctactaccttatgttacaattctcttaacccttcaacaaccgttttctcaagaacccatttctcagttcccttgaataaagagcttccactggatgcttcaccttttgttgttggctataactgtggtgtaggatgcagaacaaggaagcaaaggaagaaagtgatgcatgtgaagtgtgcagtggtggaggctccaccaggtgtttcaccctcagcaaaagatgggaatggaaccaccccttcgaagaagcagcttcgtatacttgtggctggtggagggattggagggttggtttttgctttggctgcaaagagaaaggggtttgaggtgatggtgtttgagaaggacttgagtgctataagaggggagggacagtataggggtccaattcagattcagagcaatgctttggctgctttggaagctattgattcagaggttgctgatgaagttatgagagttggttgcatcactggtgatagaatcaatggacttgtagatggggtttctggttcttggtaagtttaaaggttaagtacatatttgaattgaatcagtgatttgattgtcttttttctttttctttttttctttcttgttctatttttaggcatgcaatagggatactcatgctgattggtttaatatatcatttaaggtgttggaccctcaaaagctaattaggtagtttcaaccttgtggtaagtggcaactgtactcatttttggcaaatttaggacagagaaattctagcaatgagaaaagtacatggttaagggaaaagagtatattaaattattaatatcaattgagaactttgagttgacaaacttcaatatcatgaccgttgcaagaagtgccctagtaaaaaaactgtgaaatcttaacaacagcagcagggagtcaaacgcaactgcaatggaaattcagatgtttaacaatgtttgtgtcagtggtcctagttaacaagtctattaacatcctgtttgttaatgagtttgattactcttagtgagtacatccttcattttaaagttgggggaacttgggcttaataggattctgtagaactaaactaaccaaataattattctagcaaacatgcaaatgggtgttctgttattgtaaattttcctttctttcacaaacaacaatcttatttccttactatcaagttataaaaaaaaatgttttcttctattttctgtatgaaattttagcatttggtaccgtgaattgatatataggttgttgtcaggtacgtcaagtttgatacattcactcctgcagtggaacgtgggcttcctgtcacaagagttattagtcgaatggttttacaagagatccttgctcgcgcagttggggaagatatcattatgaatgccagtaatgttgttaattttgtggatgatggaaacaaggtaattagagaaagattgttaaagattctcctcatatttctgtctattatgatatctattttctgatgcttttatttctttcttattgaaatttgaaaacgcattggcagacaaatatcatttgattgatgtttactactttcccaccctgcattccacaatttcaggtaacagtagagctagagaatggtcagaaatatgaaggagatgtcttggttggagcggatggaatatggtccaaggtcttattaatttttctctgattatgatcttattctttagcaaataatttttcccatatgaaagtaaacatcatactgaaacatgacaagtactgtggagctcatatgtgtgttagatttgttctttcattgtgaagtagtgaactctttctgtcattctgtctgtaagcggttcgtataatcataaattctgcatttaattttattgtaattttttatgaataggtgaggaagcagttatttgggctcacagaagctgtttactctggttatacttgttatactggcattgcagattttgtgcctgctgacattgaaactgttgggtatgttgattatattcatgtatcgtatctctatttatccattttctgtggactatgttgaaattttgtaaacattacttacattgagttgagaagcttacttgaacttcttcgctggaagataccgagtattcttgggacacaaacaatactttgtatcttcagatgttggtgcgggaaagatgcaatggtatgcatttcacaaagaacctcctggtggtgttgatgagcccaacggtatgcattctctttgtgtttgagatttgaaaacagttagaagcacaccctttgcaaatgttaaattttcaatgtggtctacttatagacatacctttttaattgtttgtgttggcttttacataactgatgccgttttttaattgattgtacctggaaagaattttcaactgttatggtaatgaataattgattatttgtttgacattttgagcttagatcatttctatgctgaggatcactgattattatgttctgccactagttactactaataatgaacaattagtgggggttatatatgaaaaacgaaaaaaaaaggtaaagaaagctgctcatggcatcatctatttgtcggaatagaaggtattttattcaccttgttattcttgagtcatttacacagtataaatagatacaagagagagagagagagagtttaggggttgtcctagcctatacatgatactgatggctaaatataactaaatttgacccttgatctaagggatttgacagctatttacaattaatgttcttattttctggtaattctaatgactcgcgctaacactctccctcaagttagtacataaatgtctttaatgtccaacttgtcaagggagtcagggaagttcttatcggacactacctttgtaagaatgtctgtcaattggtcttccgacttgacaaacggaaatcaaataatcttcttgtcaagattttgcttgatgaaatgtctatctacctccaggtgctttgtttgatcatgctggatagaattttgtgagatatcaattgtatctttattgtcagagaacaagttcatctccatgctaggaccaaagccaatctccattagtaaccttttgagccataatagttcacaaagtcctttggtcatgcccctaaacttagcttcagcactagacaaggccactactttttgctttttacttctttgtgtaactaaattcccttccacaaatgtgaagtatcttgatgtggattttctgtctataacattccttgcccaatttgcatcagtgtacccttcaacactcaagtgattattcttagagaacatcaacccctttcttggtgaggattttaaatagcgaagtatttagtttattgcatctgtatgttcttcacttggattatgcataaattggctcaccacacttacggcataggcaatatcaaggtgagtatgtgacaaatagataagtttccccaccaatttttggtatcttcctttattcatgtgcacttggtctgtgtattgagaaagtttgtgattttggattgttggagtatctgttggtttgcagtccagcagccccaccttagtcaataagtcaaacacctactttctttgatacaagaaaatgcctttttgtgaccttgccacctttttccccaaaaaaatacttgaagtccacccaaattcttcatctcaaactcagttgtcaattgctcttgtagtcttgaaatttcttcgatatcatctccaataataatcatgtcatctacgtaaataattacagttgtcacatttccttgcagatgctttaagaatagggtgtgatttgaattactttgatcaaaatcataccttctcatagttgagctaaatcttccaaaccatgatcgaggttattctttcaatccatatagtgcttgctctaacttgcataccaccttggttccagaagagggcatatagctcgatgggatatctatataaacatcttcttcaagatctccatggagaaaagcgttcttcacgtcaacctgaagtaatgaccaatctaaatttgttgcaagagataataggactctaacaatatttagcttggccaacggtgaaaaagtctctgtataatctataccttaagtctatgtgaacccttttgccactagccttgccttgtgccgatcaatggatccatcacttttgtatttgattgagaacacccatttgcaccccaatatcttctttaccttaggaagtggtatcaaccttcatgaattattgttttgtaaagcttccatttcctcttgtatggctagtgcccacttaggatcagctaaggtttcttccacattttttggaatatgacaggataataatgtctttatgaaggcatgaataggatttgataaatgttgagtagacacataatttgcaatggggtatttggatcgcctttcttctacattagggcagtatctattgggtggctttcctcgattgttcctgataggcaatacatatcttacagaagcattagtagatgaggagatagtaggagagcttacctcaggattgttccaagtgagagggtcgggtattatgaaaagagggggctcttgacttccttgatcgatatagtcttcattgacgggaacttcttcactagcagttccttcattagtagaaggatcctctttacactcaacttcagttatatttaaatgttgcatttctataggttcatcatggattactccttcaacttcagattcatgctctccaatttgtttcttttcaatagctggtgatggagccaccaagtaccaatttagctctctattcatggactccccttgaagagctgaattggataccataggagaaaagaaaggttcatattctagctaggaaagttacatccatggtgatataagttttttcgatggtagggtccaagcatttgtagcattttttgtgtagtccatagcccaaaaacaagcacctaacaacacatgcatcaagtttggtgcgttggtttttgtggagatgaacaaaaaccacacacacaaaaatcctaggttaaaacatcaacacagtaggtaattgtacatgagaagagagtacttgcagaggtgttttaaaatgcaagaccttggagggcatgcagttgaggaggtatacaactgcaattacagcatcatcccaatggtgagttggaacatgggtgcaaagaagaaaagcacgtgttgtctctaagatatgacgatttttttgttcggcgacactattttgttgtggagtttgtgagcatgaggtctcctgaaagagaccatgttgttgggattatgctttgaaatgatggttaacaaactccattatatgaccgaagaacttgaattttggttgaaaactgattttgaatcatgatatgaaaattctgaaatatttgaaacacctcatctttagtcttcattaagtaaagccatgtcattcgtgtgcaatcatcgacaaatattacgaaccaacggtgaccaaatacgatagtcctaagggaaggtccccacacatcaaaatgttttagagcaaaaggaacactgcttttattcaaactcatacggtaggaggttctatggcttttagccaaaatacaagtcacacatgtaaacctaacatttttcaaatgtaaaaataaatatggaaataagtgtcttaaataactgaatgatggatgtcccaaatgacgatgccacaaccatatttctcattctttgctactagattgatggcacacgtgatgtgcttctcctttgctaaaattgtctaggtagtatagcccccctctcttagtaccacgcccaataatcttcttgttgagaacatcctgaagaaggaaaaaaagtagagtacatcaataccacataattgagctcttcagttatttgactcatagacattaatttattagataatgtaggaacgaggaaagtgtgagataaagagactgagggtgataatgccacagttccaacacctacgattggctatgcaactctatttgcattcgcaatgcaagtcctatgtggtggagttgactgggagaagttcatgggatcaaaggtcatatggtttgttgccccagagtcaataatccatgcacttgcatcctgagagttagaactacagaaaacttgacgccagttacctggtttcattgaggtggaggaactagttacaggacaaagcgagagttgagactcaacagtgactacaacagctttaccagtgccttcctcaagtgtcgtggtgtcttttttcttcttagcttgcaactcatgccactattttggatacccatgtagcttgaaacaggtatcacgattgtgtctaacatttccacaacgtgtgcactttcctccatcagatggacccttcgacttcaagactgagttgtgtttcccagccactagtgttggcaattggccaaattttgaacctttagtggccataatagcatcacttgttgtattatctaccccagatgccattatagtctaacaagtgtcctcccttcgaacatgtgcataagcatgctcgattgtgggaaatggcttcagttggagtatgtcactccaaatgttatcgagttggtcatccaatccattgagaaagatataaactctttcctcttgtagtaaataattgtatttttgtatatctcttgggcactccatggcattagggcaccgaaagtctatctcacgccacagcccttgtagaacattgtaatatttttcaattaatccacttttctacgacatctgggttaccttacgtcgaaggttcctaaagttgtgaagtatcagatccatcaaaatatgtcatagcaatggagtcccaaacccctttggctgttgggaagcggatgaaattccccaccagtgagggatccattgagttgatcaaccatcctttcactatagcgttctcagttcgccatttacagaatgtcgaatcaatttccgacgacaaagggaatgatccgtcaatatatcacagtttgtctttgcctgagatgtacatctcagccacctgggaccatagggcatagttggaaccatccaacttgatcctgatcgatatcgtcgaggaattagagattgacagttgtgatgttgactaggcttttgacaaaacctccatcatttttgtcgtaagatttgccaaaatggaatcggtagcggaagcatttgatggctactttgtggaatcaccaatcgctgaggaatcaaccatctttgggtcccaataatatgctcttgataccatgtcagaacataaggtattttattcaccttgttattcctttgagtcatttacagagtataaatagatacaagagggagagagcttaggagttgtcctagcctatacacgatactcatggctaaatataagtaaatttgacccttgatctaagagatttgacagctatttacaattaatgttcttattttctggtaattctaatgactcacgctaacattttttagcatttcaggaagcatgcttcatacctacaaaatactgatacttgcatttgtttcatgtatcgatattagacatgtatctgatattgatacttttggatacttcaccaataataagtatcagaaaactatcaagtgttagatttaactaactacatcaaatctcaattttaaagtgggtcacatttttactagttttaggaagaaatgaagtagctactattcttgtttgtaggaaataggctatgatttatgatgactatgattcatagtttctattttgtttttagtaatgttcaaaaagtgtgcttttagtttgaatttgtagagtaacaatgttatattatgtttaatattttatgcacacatgtatgtgtatcttctacattacgtatctgacattaagaataattgtatcatatccaatatgtatccatgcatcatagtagttagcatggtatttatttgagattacctcatgattgctttttgttaatattcaaagatggacctaaatgcttctctcgcccattctatctcagactcatttccttctattactgaatctttatccatctttcttgacgtggaatagaatggattgtaaatatcaagcattcaagctggtataatgatgaagattgctaatgaaatagcctgggttctatgacagtaatttagcattggaaatgtagaaataagatgaccttgaaaatatacaaatagatgtgtatctttatttttcatccttttcaatgaggaaaaaaaagttaaattatcatattcatatctaatttcttagttctttctgcatggcaggaaaaaaggaaaggttgcttaggatatttgagggctggtgtgataatgctgtagatctgatacttgccacagaagaagaagcaattctaagacgagacatatatgacaggataccaacattgacatggggaaagggtcgcgtgactttgcttggtgattccgtccatgccatgcagccaaatatgggccaaggagggtgcatggctattgaggtatatattctttttatctggtatttagcttattcatcctctctcaatgaaatatgaagttattatgagttctacattagtttcagaaattttttacttactcagaccaggaatggatgcaagaattgactaatgatgggggtggagggggaatacacaagataaacgaaaattcttagtttaaacatatgtatcatacatgttatttcatataataaatttcatctatgattgagttagtgtcaaatttttaaacaaagcaatgaaagactatttgtaccatatgactattatatcctccaagtcttggattgtttacatactgtttactcatccaaaattacaataagaatccacttttcattgatgtgaatctatttaagcttcctctttaaatattctctttttacctgtgggtcttcttacttataaaataaaaataaaaaattgttataaattataatgtattagcccattaaacacaaacttatcaaaactgattgtagaggctatttttgttttgcattaaaatttgattacctgtaatataaaatggaaatgtaataaggtcaatagggagactcctcggctctctctacctttgtccctctttcatacctaagacattatcactgattttctggactttggtatttgaaactttttcttaactttcttgtcgttaagctttctattattttttttcttggcacagctttcaaataaacaattttatttccatctgtctgatggatatacactatttacagaaacatacataaaattaaaatatttcctactttaagaggagatggagataatagatgtcaaactatctcaacttttgttagaggctcgaaagggactggtaatcaatgaaacacttgaaaagtgaggcaaaaactccttctgactagtttgttttatcatttcaggacagttatcaacttgcatgggagttggagaatgcatgggaacaaagtattaaatcagggagtccaattgacattgattcttccctaaggaggtaatataccattttgcatttatgcaaatgcaaatgatgatattattgacttgattcgaaaagagagatcgtttcttgcatcaaccatggctcaatagttaatgtttcattattggtttaaatacatttttgtttcttggaatttagtgatcttttgtttttcgttcttgtaaaaaaaaaaaaaatttcagtcattgtaaattatattatttttttgtttttagtctgtaaaatattttagatgacgttttgaacaataaaaaatgttatataaagtattataaggacgaaaaacaaatataatttgtaatgactaaaaaataattttttacaagaataaaaaaaaacgctaaattatcatgactgaaaagatatttaagcattcatcattttcaaattggtaaatttgatgataaatctctttggtgattaatggtgcccatcttttttatcccttttgcattaatactttcagctacgagagagaaagaagactacgagttgccattattcatggaatggctagaatggcggctctcatggcttccacttacaaggcatatctgggtgttggtcttggccctttagaagtaagaacttaaaatttatcaataagagaatgaaaaatatggtgaaatgacactcaaattgaaataattaaatgcacaaattcattcttgcttcccttactctgcagtttttgactaagtttcgtataccacatcctggaagagttggaggaaggttttttgttgacatcatgatgccttctatgttgagctgggtcttaggtggcaataggtaacgtttagagcctatttttctttgctcatagtctttctttcatattgtcttcatctgatcttcttattcacatgcatgtagaaatctgagccactggataaatgatgagaactaagaagttcaagaaattgataattgaaaacaatgtttagtctttacttggacaaacaataaagaaatatttatgtgtgcaattttgttactaacaacatgaaaaatgatgctacttttgcagcgacaaacttgagggcagaccactaagttgcaggctcacagacaaagtacgttgctcaaataaagtttcttttgtactttgttgctgagaagatattgtatatctggtaatttatatgttcacatctattagacttgttgtgatgttgttgttaatagctatttgattatttatgaaggcaaatgatcagttacgtagatggtttgaagatgatgaagcactcgagcgtgctattaatggagagtatgtctctgaccaaatatttgtatcacctattacatatttcttaccacaaataagtagatcacataacttaatgttcatccttgttttctcaattatagatggattttattaccgcatggagatggaacaggtctttcaaagcctatatctttaagtcgaaatgagatgaaacccttcataatcgggtatgcctattgctacacctacttacaatgcagtggcttaatagttcttaattatcataaagataatctaatttttggtcaccaattagtgttgagaagtgtgcagttcaattcctaattatcataaagataatctaattttcactttcagcctattttctaatatgtttttgtaattgtaggagtgcaccaatgcaagataattcaggcagttcagttacaatttcttcaccacaggtagcacaaatttatcattacctgaatgaaacacggtcccatacttgatgtgcttcacatcattaacataatcaacaatttctctttttaaggtttctccaacgcatgctcgaattaactataaggatggtgccttcttcttgattgatttacggagtgagcatggcacctggatcattgagtaagtaaaagaagtgaaaggttctttgtttgacattaaaagttttaaattcttgagaaatctatttaatatcattctcattatgttttcagcaacgaaggaaagcagtaccgggtacctcctaattatcctgctcgcatccgtccatctgatgttattcagtttggttctgagaaggtgagatgagaggtcctaaaaagcagttgcagtatactcatgttacacatttgcatcttctaggacttatattaggtatatgaggtaaccactgatcaacatatgagaaagatactacaactgtttgattcactaattatgactagtttctttcacagcctataagaattgaacattattagttggatgattcagtctcattgagtagtgttacttgtacacactttcatcccacactcaattgtttaaagtaaaattattagtttattgcattgtttaaccaataccgaaatttcagttgttaattttatttaaatacttgtgggagaggtgctaaaaaattattcaagttacatttgatttcacaattcccctcatttatctgagacaaatgattcacagacataatcttttgcgtatattatctgttatccttgcctgaaaatgttcaattttagtttaaatggtgcactacttatttgatgtctcagcaaccaagttatgtgcaggtttcgttccgtgttaaggtgacaagctctgttccaagagtctcagaaaatgaaagcacactagctttgcagggagtatgactgattctgctcaattgcaatttgtaagttatggaaaaattatacagcacaaatttgctattgtatagtactatctgcattgttttagggtggggtattataccacagtctagtcatttaagatctgatatgttacatgcctatatggacatttaagagggactcttgggtataaatttgttactccactccaatactttttgtgtatgacatttgtaatttgttagagttagatttataacatgacacacataaacttgcacgtgattaatgttgaatggagtaagcaatgcctagctatgaatttttgcacttttaattgtgatttacagaatcaaactgtatagtttatcattataagaagagcaaattcaattgtcaccaaatt

SEQ ID NO: 102 - DNA, 3879 bp, synthetic construct
  source 1..3879: mol_type = other DNA
ccttattggactttagaatattgagtccatttgcaaggaaattggaagcaaatagaggcgtcgacgtgtctgttgaggttccagtgcttggcctcttctacgccgcaaggcatgtttccaactttgctcattctaaattatatctctgcccttaactactattataaactaaattttgtaacttggctaatgaaaacaagtgggtatgcaatattcttggacaattatattttccttttattatagagtctttagtattccattttgggctatgattacctctgcattctaaatgatatcctgttgatcttgttgatttggttttcacagtatcaggttacatttggagttgaagacatatacatatattaatatattattattattattatattatatgtatatatggcaacgtggggtgcgcactcaatgttgttatcccatggggaaggcactggcactcccaaagtatgtgtaaaagcaggccttacggttagaggtgatttaaggtttcacaggactagaggttttaaatatgctcctagtgtggtagtgctcaaagtgttggccaccgccagaaagccaacacggttgcgcttgttaagaccttattgcagtgtgagggggcaacacgaccatgggaaaggatatttttctcccactaattcacaaataccacgggttagtaaccgctgcttagtttatcctacttcattgttaatgcaatcatgcttatcccttttgtgaaaatgcaaaatttgttcctaagattgcacctttatatccaatgtccatcctctgagtttactttacaccttgtttggatagcaacatatattgaaattcgccgcggatcgaatgcttgcgtttctcaaagattggagtaatttacgcatcacggcagtagctggcatattggtatccgttttaatgatcattccaccagctgatgctgttgacgctctcaaaacttgtgcttgcttgttgaaggattgcaggtatgccatctttctttctcttgttcttgttctgccatgactacatgttatggccaccaatttcatactcttttgtctatgcaggatagaactggctaagtgtctttcaaacccatcttgtgcggccaatattgcttgtcttcaaacttgcaacaatagacctgatgagaccgaatgccaagttagtctcgcgctttacatcaaattcatgcctgcaccattattcatgtctccaaatcctgctcatgaagcttttattttcttcttatttttccagattaaatgtggggacctgtttgaaaacagtgtggttgatgaattcaatgaatgtgcagtatcacggaagaaatgtgtacctaagaaatctgatgtgggagaatttcctgctccaaatcctgatgtccttgttaatagctttaacattgctgagttcagtggcaagtggttcatcactagtggcttaaatcctaccttcgatacctttgactgccaattacatgaatttcacacagaatccaacaaacttgtgggtaatttgtcatggagaatacggactccagatgctggatttattactaggtccgctgagcagagatttgtacaagatccttcctatcctgggatcctttacaatcatgacaataagtaccttcactatcaagatgactggtaatttgttattcataagctttcttttcaatttttacttagctagacatgtatagttaattgcaactacatccacacgaagcacattgacgtgagggttgtgctagagccaaaataaaactagcttcctacttacttgtagagttgtagtgtaattaaggcacagaccaaatcatgtttcttaattaagttaggagagttagaaaaggttgttttgatcattatcaggagataaaaaaattcagcaaacttgaataaaaatttaattggtatgaagctcgacttaagaaattcacacacatggtagaattcatcacaaatcactatactgattctagatccaatcattgttaatggttcaattgtctactggaaaacatcctctgattcaatcaatgaaggacaacgccaatttgcaattgcaacaccaatgtttaggtccaattgatttaagaatgatccaatttaattagaccgcaaattcattagtgtagcttctaaacaaattctgaaataaattccaaaacctaatcagatctagttcaggcataaacatgcttataaacagatttggaaaagaaggggaagtttgaatccaaaatagaattaagaacaagtctcagaatccaaatcattagccttctatagcaatcaccaatggagaaactaaaaatgcataaaacgcaaacaaggggggaaaaccttgaatctgaaaggtcacaactacacaaatggtccaagtctatttttgcactatgtttttcctcttttgtaactaattatctcctaatcccaagattacccttatcccaactattagcccatgtaataagcttttcaacgatatctcatttacctaacttgcccttgaatttaatgatatatgctcaaaagacaaaaggtatgtcatgcatgctcgcccaatacttctggtctaactctaaagctgaattttaacactgaaattcccgaattcgtcctttttacaaagtttgtagatatctttattaactttcgtagcatataaagaacgtcataattagaggtctatagcttaagttatgactaaaatgctgatatagtgtcaaaatataactaatgctgtcaaaattattcactgcccaatttggcccaataattcaaattagtggtaggactaatcaatcaagaccaaaacctcataagcaaggcaattaaatatgttaaaaatagtagttatcatacaccctgtgtttgaaggaagtaaatgttcctttcctagatcagaaaaagaatacaacatgttgttccttttacgtgtgcatacattaaatcatttaacatttgccagatatttgaatcaaagagtcttttgttaacattagaagacttacaaacaatgatatatatatatatataggtatattttgtcatccaagattgagaataaatcagatgactacgtatttgtatactaccgaggcagaaacgatgcttgggatggttatggaggtgctgttgtatacacaaggagtgcagttttgcctgaatctatagttcctgaacttgaaaaagcatctaagagtgtgggaagagactttagcaagttcatcaggacagataacacatgcgggccagagccttccctggtggaaaggctggagaaaaaggtagaggaaggagaagagaccattgtaagggaagttgaacagatagaagaagaggtggagaaggtggggaaaacggaggcgaccttatttcagaagttggcagaagggttcaaaatgtttcaagaagacgaggaaaatttcttaagagggttatcaaaagaagaaatggagattcttgatgggcttaaaatggaagccggtgaagtagaaaagctctttggacgtgccttgccgattaggaaactaagataagtctgagaaccatgtccaagataacttggcatcataatctttttttatcatctttgcttttattcgttgactacatatattgcatggctgtagatagctttcctccttaatcattgcttttatgttgacatactaatgattattcaaatatttgtaacagcatttatcaattgttgtatatatattcagagtattgcatttgcataggtttagatgcttgtgctttttttcaagtcgaaaatatttggaatgggaattccgacatgaggaatgctgtatactaccagtgttcttcctcaactactaataca

(19) United States
(12) Patent Application Publication
(10) Pub. No.: US 2024/0368616 A1
(43) Pub. Date: Nov. 7, 2024

(54) MODIFIED UPSTREAM OPEN READING FRAMES FOR MODULATING NPQ RELAXATION

(71) Applicant: The Board of Trustees of the University of Illinois, Urbana, IL (US)

(72) Inventors: Benjamin HAAS, Urbana, IL (US); Steven James BURGESS, Urbana, IL (US)

(73) Assignee: The Board of Trustees of the University of Illinois, Urbana, IL (US)

(21) Appl. No.: 18/654,999

(22) Filed: May 3, 2024

Related U.S. Application Data
(60) Provisional application No. 63/500,518, filed on May 5, 2023.

(51) Int. Cl. C12N 15/82; C07K 14/415; C12N 9/02; C12N 9/22; C12Q 1/6897

(52) U.S. Cl. CPC C12N 15/8269; C07K 14/415; C12N 9/0055; C12N 9/0073; C12N 9/22; C12Q 1/6897; C12Y 110/99003; C12Y 114/1309

(57) ABSTRACT

The present disclosure relates to genetically modified plants including one or more edited open reading frames (uORFs) in endogenous nucleotide sequences encoding photosystem II subunit S (PsbS), zeaxanthin epoxidase (ZEP), or violaxanthin de-epoxidase (VDE). The present disclosure further relates to methods of producing the genetically modified plants, as well as to isolated constructs and expression cassettes for use in producing the genetically modified plants. In addition, the present disclosure relates to transient screening methods to study gene expression.

### CROSS-REFERENCE TO RELATED APPLICATIONS

This application claims the benefit of U.S. Provisional Application No. 63/500,518, filed May 5, 2023, which is hereby incorporated by reference in its entirety.

### REFERENCE TO AN ELECTRONIC SEQUENCE LISTING

The content of the electronic sequence listing (794542002500seqlist.xml; Size: 176,096 bytes; and Date of Creation: Apr. 8, 2024) is herein incorporated by reference in its entirety.

### TECHNICAL FIELD

The present disclosure relates to genetically modified plants including one or more edited open reading frames (uORFs) in endogenous nucleotide sequences encoding photosystem II subunit S (PsbS), zeaxanthin epoxidase (ZEP), or violaxanthin de-epoxidase (VDE). The present disclosure further relates to methods of producing the genetically modified plants, as well as to isolated constructs and expression cassettes for use in producing the genetically modified plants. In addition, the present disclosure relates to transient screening methods to study gene expression.

### BACKGROUND

Light intensity in plant canopies is dynamic, and leaves routinely experience sharp fluctuations in levels of absorbed irradiance. When light intensity is too high, or increases too fast for photochemistry to utilize the absorbed energy, photoprotective mechanisms are induced to protect the photosynthetic antenna complexes from over-excitation. One of these photoprotective mechanisms is non-photochemical quenching of chlorophyll fluorescence (NPQ; Müller et al. Plant Physiol. Vol 125, 1558-1566, 2000), which is an inducible protective process that dissipates excess absorbed light energy in the photosystem II (PSII) antenna complex as heat. Changes in NPQ can be fast but not instantaneous, and therefore lag behind fluctuations in absorbed irradiance. In particular, the rate of NPQ relaxation is considerably slower than the rate of induction, and this asymmetry is exacerbated by prolonged or repeated exposure to excessive light conditions. Due to the relatively slow relaxation kinetics of NPQ, energy that could otherwise be used for photosynthesis is lost when the plant experiences frequent sun-to-shade transitions.

Previous research has shown that overexpression of genes involved in NPQ, the photoprotective process necessary to dissipate excess absorbed light energy, can increase the efficiency of photosynthesis and biomass accumulation (Kromdijk J, Glowacka K, Leonelli L, et al. Improving photosynthesis and crop productivity by accelerating recovery from photoprotection. Science. 2016; 354(6314):857-862) of the model crop Nicotiana tabacuin in field-relevant conditions. In addition, overexpression of genes involved in NPQ has been shown to increase seed yield in soybean (Souza A. P. D., Burgess S. J., Doran L., Hansen J., Manukyan L., Maryn N., et al. (2022). Soybean photosynthesis and crop yield are improved by accelerating recovery from photoprotection. Science 377, 851-854. doi: 10.1126/science.adc9831). In each of these studies, the three NPQ genes violaxanthin de-epoxidase (VDE), photosystem II subunit S (PsbS), and zeaxanthin epoxidase (ZEP) were introduced to increase total (transgenic plus native) transcript levels, which translated into higher protein levels. While these previous approaches were successful, they relied on expression of foreign DNA, or transgenes, which incur additional regulatory complications and can be susceptible to gene silencing across generations (James V A, Avart C, Worland B, Snape J W, Vain P. The relationship between homozygous and hemizygous transgene expression levels over generations in populations of transgenic rice plants. Theor Appl Genet. 2002; 104(4):553-561. doi:10.1007/s001220100745).

CRISPR/Cas9 has dramatically expanded the capacity to produce targeted edits of endogenous genes. However, the ability to increase gene expression without the use of persistent transgenes is still lacking, and finer genome engineering is bottlenecked by low editing, transformation, and regeneration efficiencies (Wada N, Ueta R, Osakabe Y, Osakabe K. Precision genome editing in plants: state-of-the-art in CRISPR/Cas9-based genome engineering. 2020:1-12). A targeted approach for CRISPR/Cas9 editing would rapidly accelerate what is possible by gene editing in crop plants.

In describing gene structure, an open reading frame (ORF) is the part of a reading frame that has the ability to be translated. An ORF is a continuous stretch of codons that begins with a start codon (usually AUG) and ends at a stop codon (usually UAA, UAG or UGA). A protein-coding part of a mRNA is referred to as a main ORF (mORF). Upstream of the mORF are upstream ORFs (uORFs), which are not part of the translated protein. These uORFs can be short (e.g., 9 nt) or long (e.g., hundreds of nt), overlapping or non-overlapping, and in-frame or out-of-frame with the mORF. Both canonical and non-canonical start codons are effective in uORFs. The protein initiation complex (PIC) scans uORFs from the 5′ end, and if a start codon in optimal sequence context is recognized, GTP is hydrolyzed and the 80S initiation complex is formed to start translation (Hinnebusch, Alan G., Ivaylo P. Ivanov, and Nahum Sonenberg. “Translational Control by 5′-Untranslated Regions of Eukaryotic MRNAs.” Science 352, no. 6292 (Jun. 17, 2016): 1413-1416).

There are a couple of known mechanisms of uORF repression. One is that ribosomes translate the uORF and fail to re-initiate at the mORF (i.e., re-initiation is inefficient). Another is that stalling of the ribosome prevents leaky scanning of the uORF, attenuated by small molecules or the peptide itself. Studies have shown that over 30% of transcripts across 11 plant species contain uORFs (Arnim, Albrecht G. von, Qidong Jia, and Justin N. Vaughn. 2014. “Regulation of Plant Translation by Upstream Open Reading Frames.” Plant Science 214 (January): 1-12.

Doi[dot]org/10[dot]1016/j[dot]plantsci[dot]2013[dot]09[dot]006[dot]), yet direct evidence of particular uORFs inhibiting downstream ORFs is missing for most. Moreover, the occurrence and translation of uORFs is “associated with below-average translational efficiency for the downstream ORFs genome-wide” (Hinnebusch, Alan G., Ivaylo P. Ivanov, and Nahum Sonenberg. 2016. “Translational Control by 5′-Untranslated Regions of Eukaryotic MRNAs.” Science 352 (6292): 1413-16). doi[dot]org/10[dot]1126/science[dot]aad9868). Recent research utilized CRISPR/Cas9 to edit specific uORFs (Si, Xiaomin, Huawei Zhang, Yanpeng Wang, Kunling Chen, and Caixia Gao. “Manipulating Gene Translation in Plants by CRISPR-Cas9-Mediated Genome Editing of Upstream Open Reading Frames.” Nature Protocols, Jan. 8, 2020, 1-26). This editing approach showed that by disruption of specific uORFs, target genes could be effectively overexpressed.

NPQ genes (which can include one, two, or three of photosystem II subunit S (PsbS), zeaxanthin epoxidase (ZEP), or violaxanthin de-epoxidase (VDE), preferably at least both of ZEP and VDE) represent a promising avenue for genome editing approaches across plant species, as it is known that NPQ genes are found in all plants, and NPQ proteins are highly conserved in their function. There exists a need for genetic engineering approaches that alter endogenous gene expression in order to achieve agronomic gains without the need for stable transgenes. In particular, there exists a need for such approaches able to augment photoprotection (e.g., by targeting specific NPQ components) in order to improve photosynthetic processes and ultimately plant yield.

### BRIEF SUMMARY

In order to meet these needs, the present disclosure provides targeted disruption of specific uORFs in NPQ genes to overexpress the NPQ genes without the need for stable transgenes. In particular, the present disclosure provides targeted disruption of uORFs in the NPQ genes photosystem II subunit S (PsbS), zeaxanthin epoxidase (ZEP), or violaxanthin de-epoxidase (VDE). Disruption of specific uORFs of these genes in both Glycine max (soybean) and Vigna unguiculata (cowpea), as exemplary plant species, results in overexpression of these genes, demonstrating that this approach is effective for multiple genes and across species. The present disclosure further provides transient screening methods to study gene expression that utilize dual-fluorescence expression vectors.

An aspect of the disclosure includes a genetically modified plant including one or more edited endogenous nucleotide sequences encoding a photosystem II subunit S (PsbS) polypeptide, a zeaxanthin epoxidase (ZEP) polypeptide, or a violaxanthin de-epoxidase (VDE) polypeptide, wherein the one or more edited nucleotide sequences are edited at one or more upstream open reading frames (uORFs) in a transcript leader sequence (TLS). In a further embodiment of this aspect, the edits at one or more uORFs increase expression of the downstream main open reading frame (mORF) encoding the PsbS polypeptide, the ZEP polypeptide, and/or the VDE polypeptide. In a further embodiment of this aspect, which may be combined with any of the preceding embodiments, the edited endogenous nucleotide sequences were edited at all uORFs in a TLS. In an additional embodiment of this aspect, which may be combined with any of the preceding embodiments, the edited endogenous nucleotide sequences were edited at two or more, three or more, four or more, five or more, six or more, seven or more, eight or more, or nine uORFs in a TLS. In still another embodiment of this aspect, which may be combined with any of the preceding embodiments, (i) the editing removed uORF repression, increased efficiency of mORF translation re-initiation, removed stalling of a ribosome, removed inhibition of translation of downstream ORFs, or increased translational efficiency of downstream ORFs; (ii) the editing resulted in disruption of an uORF start codon, and wherein disruption was achieved by replacement of the uORF start codon with a nucleotide triplet, deletion of the third base of the uORF start codon, a frameshift mutation to disrupt an uORF reading frame, insertion of one or more nucleotides in the uORF, or deletion of one or more nucleotides in the uORF; (iii) uORF repression is reduced or removed, efficiency of mORF translation re-initiation is increased, stalling of a ribosome is reduced or removed, inhibition of translation of downstream ORFs is reduced or removed, or translational efficiency of downstream ORFs is increased; (iv) an uORF start codon is disrupted; and/or (v) the uORF start codon was replaced with a nucleotide triplet, deletion of the third base of the uORF start codon, a frameshift mutation to disrupt an uORF reading frame, insertion of one or more nucleotides in the uORF, or deletion of one or more nucleotides in the uORF. In yet another embodiment of this aspect, which may be combined with any of the preceding embodiments, the genetically modified plant includes edited endogenous nucleotide sequences encoding the ZEP polypeptide and the VDE polypeptide or the PsbS polypeptide and the ZEP polypeptide. An additional embodiment of this aspect, which may be combined with any of the preceding embodiments, includes edited endogenous nucleotide sequences encoding the PsbS polypeptide, the ZEP polypeptide, and the VDE polypeptide.

Still another embodiment of this aspect, which may be combined with any of the preceding embodiments, includes further genetic modifications that increase expression of one or more of the PsbS polypeptide, the ZEP polypeptide, or the VDE polypeptide; wherein the expression of the PsbS polypeptide is increased by expressing a transfected nucleotide sequence encoding the PsbS polypeptide in the genetically modified plant, by a genetic modification in a promoter of the edited endogenous nucleotide sequence encoding the PsbS polypeptide and/or by a genetic modification in a promoter of an endogenous nucleotide sequence encoding the PsbS polypeptide; wherein the expression of the ZEP polypeptide is increased by expressing a transfected nucleotide sequence encoding the ZEP polypeptide in the genetically modified plant, by a genetic modification in a promoter of the edited endogenous nucleotide sequence encoding the ZEP polypeptide, and/or by a genetic modification in a promoter of an endogenous nucleotide sequence encoding the ZEP polypeptide; and/or wherein the expression of the VDE polypeptide is increased by expressing a transfected nucleotide sequence encoding the VDE polypeptide in the genetically modified plant, by a genetic modification in a promoter of the edited endogenous nucleotide sequence encoding the VDE polypeptide, and/or by a genetic modification in a promoter of an endogenous nucleotide sequence encoding the VDE polypeptide. In yet another embodiment of this aspect, the expression of the PsbS polypeptide is increased by expressing the transfected nucleotide sequence encoding the PsbS polypeptide, and wherein the transfected nucleotide sequence is operably linked to at least one expression control sequence; wherein the expression of the VDE polypeptide is increased by expressing the transfected nucleotide sequence encoding the VDE polypeptide, and wherein the transfected nucleotide sequence is operably linked to at least one expression control sequence; and/or wherein the expression of the ZEP polypeptide is increased by expressing the transfected nucleotide sequence encoding the ZEP polypeptide, and wherein the transfected nucleotide sequence is operably linked to at least one expression control sequence. In still another embodiment of this aspect, which may be combined with any of the preceding embodiments that has increased expression by expressing a transfected nucleotide sequence and/or by a genetic modification in a promoter of a nucleotide sequence, the one or more edited endogenous nucleotide sequences encodes the ZEP polypeptide, and wherein the expression of the VDE polypeptide is increased by expressing the transfected nucleotide sequence encoding the VDE polypeptide; wherein the one or more edited endogenous nucleotide sequences encodes the VDE polypeptide, and wherein the expression of the ZEP polypeptide is increased by expressing the transfected nucleotide sequence encoding the ZEP polypeptide; wherein the one or more edited endogenous nucleotide sequences encodes the PsbS polypeptide, and wherein the expression of the ZEP polypeptide is increased by expressing the transfected nucleotide sequence encoding the ZEP polypeptide; wherein the one or more edited endogenous nucleotide sequences encodes the ZEP polypeptide, and wherein the expression of the PsbS polypeptide is increased by expressing the transfected nucleotide sequence encoding the PsbS polypeptide. In a further embodiment of this aspect, which may be combined with any of the preceding embodiments that has the edits at one or more uORFs increasing expression of the downstream mORF encoding the PsbS polypeptide, the ZEP polypeptide, and/or the VDE polypeptide, the expression of both the ZEP polypeptide and the VDE polypeptide are increased under the same conditions as compared to a control plant or the expression of both the PsbS polypeptide and the ZEP polypeptide are increased as compared to a control plant grown under the same conditions. In yet another embodiment of this aspect, which may be combined with any of the preceding embodiments that has the edits at one or more uORFs increasing expression of the downstream mORF encoding the PsbS polypeptide, the ZEP polypeptide, and/or the VDE polypeptide, the expression of all three of the ZEP polypeptide, the PsbS polypeptide, and the VDE polypeptide are increased as compared to a control plant grown under the same conditions. In still another embodiment of this aspect, which may be combined with any of the preceding embodiments that has increased expression of two or more of the ZEP polypeptide, the PsbS polypeptide, and the VDE polypeptide under the same conditions, the conditions are fluctuating light conditions. In a further embodiment of this aspect, which may be combined with any of the preceding embodiments, the editing was done using a gene editing technique selected from a transcription activator-like effector nuclease (TALEN) gene editing technique, a clustered Regularly Interspaced Short Palindromic Repeat (CRISPR/Cas) gene editing technique, a zinc-finger nuclease (ZFN) gene editing technique, or combinations of the foregoing. In an additional embodiment of this aspect, the gene editing technique was a clustered Regularly Interspaced Short Palindromic Repeat (CRISPR/Cas) gene editing technique, and wherein the Cas enzymes were selected from the group of Cas9, Cas12, Cas12a, Cas13, Cas14, CasX, or CasY.

In still another embodiment of this aspect, which may be combined with any of the preceding embodiments, the plant is a crop plant, a model plant, a monocotyledonous plant, a dicotyledonous plant, a plant with Crassulacean acid metabolism (CAM) photosynthesis, a plant with C3 photosynthesis, a plant with C4 photosynthesis, an annual plant, a greenhouse plant, a horticultural flowering plant, a perennial plant, a switchgrass plant, a maize plant, a biomass plant, an Arabidopsis thaliana plant, a tobacco (Nicotiana tabacum) plant, a rice (Oryza sativa) plant, a corn (Zea mays) plant, a sorghum (Sorghum bicolor) (sweet sorghum or grain sorghum) plant, a soybean (Glycine max) plant, a cowpea (Vigna unguiculata) plant, a poplar (Populus spp.) plant, a eucalyptus (Eucalyptus spp.) plant, a cassava (Manihot esculenta) plant, a barley (Hordeum vulgare) plant, a potato (Solanum tuberosum) plant, a sugarcane (Saccharum spp.) plant, an alfalfa (Medicago sativa) plant, a Miscanthus plant, an energy cane plant, an elephant grass plant, a wheat plant, an oat plant, an oil palm plant, a safflower plant, a sesame plant, a flax plant, a cotton plant, a sunflower plant, a Camelina plant, a Brassica napus plant, a Brassica carinata plant, a Brassica juncea plant, a pearl millet plant, a foxtail millet plant, an other grain plant, an oilseed plant, a vegetable crop plant, a forage crop plant, an industrial crop plant, or a woody crop plant. In a further embodiment of this aspect, the plant is a soybean (Glycine max) plant or a cowpea (Vigna unguiculata) plant. In yet another embodiment of this aspect, which may be combined with any of the preceding embodiments, the plant has improved growth under fluctuation light conditions as compared to the control plant grown under the same fluctuating light conditions; the plant has increased lutein under fluctuating light conditions as compared to the control plant grown under the same fluctuating light conditions; the plant has increased photosynthetic efficiency under fluctuating light conditions as compared to the control plant grown under the same fluctuating light conditions; the plant has improved photoprotection efficiency under fluctuating light conditions as compared to the control plant grown under the same fluctuating light conditions; the plant has an increased rate of relaxation of non-photochemical quenching (NPQ) under fluctuating light conditions as compared to the control plant grown under the same fluctuating light conditions; and/or the plant has improved quantum yield and CO₂ fixation under fluctuating light conditions as compared to the control plant grown under the same fluctuating light conditions.

Some aspects of the present disclosure relate to a genetically modified plant including one or more edited uORFs of an endogenous PsbS gene, one or more edited uORFs of an endogenous ZEP gene, one or more edited uORFs of an endogenous VDE gene, or a combination thereof. In a further embodiment of this aspect, the one or more edited uORFs increase expression of the downstream mORF of the endogenous PsbS gene, the endogenous ZEP gene, and/or the endogenous VDE gene. An additional embodiment of this aspect, which may be combined with any of the preceding embodiments, includes further genetic modifications that increase expression of one or more of the PsbS polypeptide, the ZEP polypeptide, or the VDE polypeptide; wherein the expression of the PsbS polypeptide is increased by expressing a transfected nucleotide sequence encoding the PsbS polypeptide in the genetically modified plant, by a genetic modification in a promoter of the edited endogenous nucleotide sequence encoding the PsbS polypeptide and/or by a genetic modification in a promoter of an endogenous nucleotide sequence encoding the PsbS polypeptide; wherein the expression of the ZEP polypeptide is increased by expressing a transfected nucleotide sequence encoding the ZEP polypeptide in the genetically modified plant, by a genetic modification in a promoter of the edited endogenous nucleotide sequence encoding the ZEP polypeptide, and/or by a genetic modification in a promoter of an endogenous nucleotide sequence encoding the ZEP polypeptide; and/or wherein the expression of the VDE polypeptide is increased by expressing a transfected nucleotide sequence encoding the VDE polypeptide in the genetically modified plant, by a genetic modification in a promoter of the edited endogenous nucleotide sequence encoding the VDE polypeptide, and/or by a genetic modification in a promoter of an endogenous nucleotide sequence encoding the VDE polypeptide. In an additional embodiment of this aspect, the expression of the PsbS polypeptide is increased by expressing the transfected nucleotide sequence encoding the PsbS polypeptide, and wherein the transfected nucleotide sequence is operably linked to at least one expression control sequence; wherein the expression of the VDE polypeptide is increased by expressing the transfected nucleotide sequence encoding the VDE polypeptide, and wherein the transfected nucleotide sequence is operably linked to at least one expression control sequence; and/or wherein the expression of the ZEP polypeptide is increased by expressing the transfected nucleotide sequence encoding the ZEP polypeptide, and wherein the transfected nucleotide sequence is operably linked to at least one expression control sequence. In yet another embodiment of this aspect, which may be combined with any of the preceding embodiments that has the one or more edited uORFs increasing expression of the downstream mORF of the endogenous PsbS gene, the endogenous ZEP gene, and/or the endogenous VDE gene, the expression of both the ZEP polypeptide and the VDE polypeptide are increased under the same conditions as compared to a control plant or the expression of both the PsbS polypeptide and the ZEP polypeptide are increased as compared to a control plant grown under the same conditions. In a further embodiment of this aspect, which may be combined with any of the preceding embodiments that has the one or more edited uORFs increasing expression of the downstream mORF of the endogenous PsbS gene, the expression of all three of the ZEP polypeptide, the PsbS polypeptide, and the VDE polypeptide are increased as compared to a control plant grown under the same conditions. In still another embodiment of this aspect, which may be combined with any of the preceding embodiments that has increased expression of two or more of the ZEP polypeptide, the PsbS polypeptide, and the VDE polypeptide under the same conditions, the conditions are fluctuating light conditions. In still another embodiment of this aspect, which may be combined with any of the preceding embodiments, the plant has improved growth under fluctuation light conditions as compared to a control plant grown under the same fluctuating light conditions; the plant has increased lutein under fluctuating light conditions as compared to a control plant grown under the same fluctuating light conditions; the plant has increased photosynthetic efficiency under fluctuating light conditions as compared to a control plant grown under the same fluctuating light conditions; the plant has improved photoprotection efficiency under fluctuating light conditions as compared to a control plant grown under the same fluctuating light conditions; the plant has an increased rate of relaxation of non-photochemical quenching (NPQ) under fluctuating light conditions as compared to a control plant grown under the same fluctuating light conditions; and/or the plant has improved quantum yield and CO₂ fixation under fluctuating light conditions as compared to a control plant grown under the same fluctuating light conditions. In yet another embodiment of this aspect, which may be combined with any of the preceding embodiments, the plant is a soybean (Glycine max) plant or a cowpea (Vigna unguiculata) plant.

Further aspects of the disclosure include a plant part, plant cell, or seed of the genetically modified plant of any one of the preceding embodiments.

An additional aspect of the disclosure includes methods of producing the genetically modified plant of any one of the preceding embodiments, including: (a) providing a plant, plant part, plant cell, tissue, or other explant including an endogenous PsbS gene, an endogenous ZEP gene, and/or an endogenous VDE gene; (b) selecting one or more uORFs in a TLS of the endogenous PsbS gene, the endogenous ZEP gene, and/or the endogenous VDE gene for editing; and (c) using a gene editing technique to edit the one or more uORFs of the endogenous PsbS gene, the endogenous ZEP gene, and/or the endogenous VDE gene in the plant, plant part, plant cell, tissue, or other explant, and optionally regenerating the plant cell, tissue, or other explant into a genetically altered plantlet that is grown into a plant, to produce a genetically modified plant including one or more edited PsbS, ZEP, and/or VDE genes. In another embodiment of this aspect, the edits at one or more uORFs increase expression of the downstream mORF of the endogenous PsbS gene, the endogenous ZEP gene, and/or the endogenous VDE gene. In still another embodiment of this aspect, which may be combined with any of the preceding embodiments, the edited endogenous genes are edited at all uORFs in a TLS. In a further embodiment of this aspect, which may be combined with any of the preceding embodiments, the edited endogenous genes are edited at two or more, three or more, four or more, five or more, six or more, seven or more, eight or more, or nine uORFs in a TLS. In a further embodiment of this aspect, the editing removes uORF repression, increases efficiency of mORF translation re-initiation, removes stalling of a ribosome, removes inhibition of translation of downstream ORFs, or increases translational efficiency of downstream ORFs. In an additional embodiment of this aspect, which may be combined with any of the preceding embodiments, the editing results in disruption of an uORF start codon, and wherein disruption is achieved by replacement of the uORF start codon with a nucleotide triplet, deletion of the third base of the uORF start codon, a frameshift mutation to disrupt a uORF reading frame, insertion of one or more nucleotides, or deletion of one or more nucleotides. Yet another embodiment of this aspect, which may be combined with any of the preceding embodiments, optionally further includes increasing expression of one or more of the PsbS polypeptide, the VDE polypeptide, or the ZEP polypeptide before step (c) or in step (c), wherein the expression of the PsbS polypeptide is increased by expressing a transfected nucleotide sequence encoding the PsbS polypeptide in the genetically modified plant, by a genetic modification in a promoter of the edited endogenous nucleotide sequence encoding the PsbS polypeptide and/or by a genetic modification in a promoter of an endogenous nucleotide sequence encoding the PsbS polypeptide; wherein the expression of the ZEP polypeptide is increased by expressing a transfected nucleotide sequence encoding the ZEP polypeptide in the genetically modified plant, by a genetic modification in a promoter of the edited endogenous nucleotide sequence encoding the ZEP polypeptide, and/or by a genetic modification in a promoter of an endogenous nucleotide sequence encoding the ZEP polypeptide; and/or wherein the expression of the VDE polypeptide is increased by expressing a transfected nucleotide sequence encoding the VDE polypeptide in the genetically modified plant, by a genetic modification in a promoter of the edited endogenous nucleotide sequence encoding the VDE polypeptide, and/or by a genetic modification in a promoter of an endogenous nucleotide sequence encoding the VDE polypeptide. In a further embodiment of this aspect, the expression of the PsbS polypeptide is increased by expressing the transfected nucleotide sequence encoding the PsbS polypeptide, and wherein the transfected nucleotide sequence is operably linked to at least one expression control sequence; wherein the expression of the VDE polypeptide is increased by expressing the transfected nucleotide sequence encoding the VDE polypeptide, and wherein the transfected nucleotide sequence is operably linked to at least one expression control sequence; and/or wherein the expression of the ZEP polypeptide is increased by expressing the transfected nucleotide sequence encoding the ZEP polypeptide, and wherein the transfected nucleotide sequence is operably linked to at least one expression control sequence. In yet another embodiment of this aspect, which may be combined with any of the preceding embodiments that has the one or more edited uORFs increasing expression of the downstream mORF of the endogenous PsbS gene, the endogenous ZEP gene, and/or the endogenous VDE gene, the expression of both the ZEP polypeptide and the VDE polypeptide are increased under the same conditions as compared to a control plant or the expression of both the PsbS polypeptide and the ZEP polypeptide are increased as compared to a control plant grown under the same conditions. In a further embodiment of this aspect, which may be combined with any of the preceding embodiments that has the one or more edited uORFs increasing expression of the downstream mORF of the endogenous PsbS gene, the expression of all three of the ZEP polypeptide, the PsbS polypeptide, and the VDE polypeptide are increased as compared to a control plant grown under the same conditions. In still another embodiment of this aspect, which may be combined with any of the preceding embodiments that has increased expression of two or more of the ZEP polypeptide, the PsbS polypeptide, and the VDE polypeptide under the same conditions, the conditions are fluctuating light conditions. In still another embodiment of this aspect, which may be combined with any of the preceding embodiments, the editing is done using a gene editing technique selected from a transcription activator-like effector nuclease (TALEN) gene editing technique, a clustered Regularly Interspaced Short Palindromic Repeat (CRISPR/Cas) gene editing technique, a zinc-finger nuclease (ZFN) gene editing technique, or combinations of the foregoing. In a further embodiment of this aspect, the gene editing technique is a clustered Regularly Interspaced Short Palindromic Repeat (CRISPR/Cas) gene editing technique. In yet another embodiment of this aspect, the Cas enzymes are selected from the group of Cas9, Cas12, Cas12a, Cas13, Cas14, CasX, or CasY. In yet another embodiment of this aspect, which may be combined with any of the preceding embodiments, the genetically modified plant produced in step (c): has improved growth under fluctuation light conditions as compared to a control plant grown under the same fluctuating light conditions; has increased lutein under fluctuating light conditions as compared to a control plant grown under the same fluctuating light conditions; has increased photosynthetic efficiency under fluctuating light conditions as compared to a control plant grown under the same fluctuating light conditions; has improved photoprotection efficiency under fluctuating light conditions as compared to a control plant grown under the same fluctuating light conditions; has an increased rate of relaxation of non-photochemical quenching (NPQ) under fluctuating light conditions as compared to a control plant grown under the same fluctuating light conditions; and/or has improved quantum yield and CO₂ fixation under fluctuating light conditions as compared to a control plant grown under the same fluctuating light conditions. In still another embodiment of this aspect, which may be combined with any of the preceding embodiments, the plant is a crop plant, a model plant, a monocotyledonous plant, a dicotyledonous plant, a plant with Crassulacean acid metabolism (CAM) photosynthesis, a plant with C3 photosynthesis, a plant with C4 photosynthesis, an annual plant, a greenhouse plant, a horticultural flowering plant, a perennial plant, a switchgrass plant, a maize plant, a biomass plant, an Arabidopsis thaliana plant, a tobacco (Nicotiana tabacum) plant, a rice (Oryza sativa) plant, a corn (Zea mays) plant, a sorghum (Sorghum bicolor) (sweet sorghum or grain sorghum) plant, a soybean (Glycine max) plant, a cowpea (Vigna unguiculata) plant, a poplar (Populus spp.) plant, a eucalyptus (Eucalyptus spp.) plant, a cassava (Manihot esculenta) plant, a barley (Hordeum vulgare) plant, a potato (Solanum tuberosum) plant, a sugarcane (Saccharum spp.) plant, an alfalfa (Medicago sativa) plant, a Miscanthus plant, an energy cane plant, an elephant grass plant, a wheat plant, an oat plant, an oil palm plant, a safflower plant, a sesame plant, a flax plant, a cotton plant, a sunflower plant, a Camelina plant, a Brassica napus plant, a Brassica carinata plant, a Brassica juncea plant, a pearl millet plant, a foxtail millet plant, an other grain plant, an oilseed plant, a vegetable crop plant, a forage crop plant, an industrial crop plant, or a woody crop plant. In a further embodiment of this aspect, the plant is a soybean (Glycine max) plant or a cowpea (Vigna unguiculata) plant.

A further aspect of the disclosure includes an expression vector or isolated DNA molecule including one or more gene editing components that target one or more uORF sequences of an endogenous PsbS gene, wherein the uORF sequence is edited by the one or more gene editing components to remove uORF translational repression.

An additional aspect of the disclosure includes an expression vector or isolated DNA molecule including one or more gene editing components that target one or more uORF sequences of an endogenous VDE gene, wherein the uORF sequence is edited by the one or more gene editing components to remove uORF translational repression.

Yet another aspect of the disclosure includes an expression vector or isolated DNA molecule including one or more gene editing component that target one or more uORF sequences of an endogenous ZEP gene, wherein the uORF sequence is edited by the one or more gene editing components to remove uORF translational repression.

In a further embodiment of any of the preceding aspects that have an expression vector or isolated DNA molecule, the one or more gene editing components are selected from the group of a ribonucleoprotein complex that targets the one or more uORF sequences; a vector including a TALEN protein encoding sequence, wherein the TALEN protein targets the one or more uORF sequences; a vector including a ZFN protein encoding sequence, wherein the ZFN protein targets the one or more uORF sequences; an oligonucleotide donor (ODN), wherein the ODN targets the one or more uORF sequences; or a vector including a CRISPR/Cas enzyme encoding sequence and a targeting sequence, wherein the targeting sequence targets the one or more uORF sequences.

Some aspects of the present disclosure relate to a bacterial cell or an Agrobacterium cell including the expression vector or isolated DNA molecule of any of the preceding embodiments.

Further aspects of the present disclosure relate to a composition or kit including the expression vector or isolated DNA molecule of any of the preceding embodiments, or the bacterial cell or the Agrobacterium cell of the preceding embodiment.

Additional aspects of the present disclosure relate to genetically modified plant, plant part, plant cell, or seed including the expression vector or isolated DNA molecule of any of the preceding embodiments.

Further aspects of the present disclosure relate to a composition or kit including the genetically modified plant of any of the preceding embodiments, the genetically modified plant, plant part, plant cell, or seed of the preceding embodiment, or the genetically modified plant produced by the method of any of the preceding embodiments.

Still further aspects of the present disclosure relate to methods of increasing the rate of relaxation of NPQ in a plant, including: introducing a genetic alteration via the expression vector or isolated DNA molecule of any one of the preceding embodiments, or a combination thereof, to a cell. In a further embodiment of this aspect, the cell is a plant cell.

Yet another aspect of the disclosure includes transient screening methods to study gene expression, including: a) providing one or more dual-fluorescence expression vectors including a test insert to produce one or more test vectors and at least one dual-fluorescence expression vector including a control insert to produce at least one control vector, wherein the dual-fluorescence expression vectors include a first fluorescent reporter and a second fluorescent reporter; b) introducing the one or more test vectors into a first leaf tissue and introducing the at least one control vector into a second leaf tissue, wherein the first leaf tissue and the second leaf tissue are of the same plant variety; d) excising leaf portions from the first leaf tissue and from the second leaf tissue 3 days post injection; e) placing the leaf portions abaxially in a multiwell plate; and f) measuring fluorescence on a monochromator-based plate reader in two separate channels, wherein a first channel is used to obtain a signal from the first fluorescent reporter and a second channel is used to obtain a signal from the second fluorescent reporter. In a further embodiment of this aspect, the plant variety is an N. benthamiana variety. In an additional embodiment of this aspect, which may be combined with any of the preceding embodiments, step b) includes: b-1) transforming a first Agrobacterium tumefaciens strain with the one or more test vectors and transforming a second A. tumefaciens strain with the at least one control vector; and b-2) injecting the first A. tumefaciens strain harboring the one or more test vectors into the first leaf tissue and injecting the second A. tumefaciens strain into the second leaf tissue. In yet another embodiment of this aspect, which may be combined with any of the preceding embodiments, the leaf portions are discs. In still another embodiment of this aspect, which may be combined with any of the preceding embodiments, the multiwall plate is a 96-well plate or a 384-well plate. In an additional embodiment of this aspect, which may be combined with any of the preceding embodiments, the first fluorescent reporter is a green fluorescent reporting and the second fluorescent reporter is a red fluorescent reporter. In yet another embodiment of this aspect, the first fluorescent reporter is mNeonGreen and the second fluorescent reporter is tdTomato. Still another embodiment of this aspect, which may be combined with any of the preceding embodiments, further includes step (g) normalizing the signal obtained from the first fluorescent reporter in step (f) to the signal obtained from the second fluorescent reporter in step (f) to reduce noise, and to produce a graph of normalized results comparing results obtained from the one or more test vectors with results obtained from the at least one control vector to determine expression differences between the one or more test vectors and the at least one control vector. In a further embodiment of this aspect, comparing results further includes determining p-values using a nonparametric bootstrap for two-sample t-testing that is adjusted for multiple testing. In yet another embodiment of the aspect, which may be combined with any of the preceding embodiments, the test insert includes a mutated TLS, optionally wherein the mutated TLS is mutated in one or more uORFs, and wherein the control insert includes a wild-type TLS.

Further aspects of the present disclosure relate to a genetically altered plant genome including (i) the one or more edited endogenous nucleotide sequences in the genetically modified plant of any one of the preceding embodiments, or (ii) the one or more edited endogenous nucleotide sequences in the genetically modified plant produced by the method of any one of the preceding embodiments.

Additional aspects of the present disclosure relate to a non-regenerable part or cell of the genetically modified plant of any one of the preceding embodiments.

### BRIEF DESCRIPTION OF THE DRAWINGS

FIG. 1 shows a transient expression workflow to screen transcript leader sequences for upstream open reading frame (uORF) editing targets, from construct assembly to a comparison of fluorescence signals. In step 1, at top, dual-fluorescence expression vectors are produced that incorporate either a wild-type transcript leader sequence from a target gene with a functional upstream open reading frame (uORF) or a mutated, nonfunctional uORF sequence (AAA), with the start codon replaced or deleted. In the expression vectors, “LB” represents the left border, “35Spro” represents a Cauliflower mosaic virus (CaMV) 35S promoter, “ccdB” represents a toxin required for bacteria selection that is replaced by either the uORF or the AAA insert, “mNeonGreen” and white stars represent the coding sequence (CDS) for a green fluorescent protein variant, “Term” represents a terminator, “OCSpro” represents an Octopine synthase promoter (Agrobacterium tumefaciens), “tdTomato” (and striped boxes) represents the coding sequence (CDS) for a red fluorescent protein variant, “Term” represents a terminator, and “RB” represents the right border. In step 2, in middle, A. tumefaciens C58C1 harboring a construct is injected into 4-6-week-old Nicotiana benthamiana leaf tissue. 6 mm leaf discs (indicated by patterned circles on leaf diagram on left) are excised 3 days post infection and placed abaxially in a 96-well plate (shown on right) or processed for RT-qPCR (not shown). In step 3, at bottom, fluorescence is measured on a monochromator-based plate reader in two separate channels (shown on left). The signal obtained from mNeonGreen is normalized to the tdTomato control to reduce noise stemming from pipetting error, differences in transformation efficiency and cell viability (graph of normalized results shown on right).

FIG. 2 shows boxplots of mean-normalized fluorescence data of wild-type and mutated transcript leaders from A. thaliana BRASSINOSTEROID-INSENSITIVE 1 (AtBRI1) and L. sativa GDP-l-galactose phosphorylase 2 (LsGGP2). From top to bottom, data from the wild-type transcript leader from AtBRI1 (“AtBRI1”; white), the mutated transcript leader from AtBRI1 (“AtBRI1-AAA”; white), the wild-type transcript leader from LsGGP2 (“LsGGP2”; striped), and the mutated transcript leader from LsGGP2 (“LsGGP2-AAA”; striped) are shown. Relative fluorescence for each construct is reported in a ratio of the Relative Fluorescence Units (RFU) measured in the mNeonGreen vs. the tdTomato channel. The excitation/emission wavelengths are 490 nm/520 nm for mNeonGreen and 540 nm/595 nm for tdTomato, respectively. Obtained relative fluorescence values are normalized to the mean values of the wild-type transcript leaders, respectively. The mean relative fluorescence of the respective wild-types are highlighted by the dashed line. Values with corresponding brackets at the right of the boxplot indicate the adjusted p-values of comparisons between each transcript leader with and without uORF disruption. P-values are determined by a nonparametric bootstrap for two-sample t-testing and are adjusted for multiple testing after Benjamini & Hochberg (1995). Adjusted p-values are indicated for n=13-14.

FIGS. 3A-3C show wild-type transcript leader sequences, boxplots of transient expression assay results, and a bar plot of relative mRNA expression. FIG. 3A shows sequences of the wild-type transcript leaders of Glycine max photosystem II subunit S (GmPsbS1, Glyma.04G249700; SEQ ID NO: 22), violaxanthin de-epoxidase (GmVDE1; Glyma.19G251000; SEQ ID NO: 23) and zeaxanthin epoxidase (GmZEP3; Glyma.17G174500; SEQ ID NO: 24) as well as Vigna unguiculata photosystem II subunit S (VuPsbS; Vigun09g165900; SEQ ID NO: 25) and zeaxanthin epoxidase (VuZEP1, Vigun03g277500; SEQ ID NO: 26). Start-codons of uORFs of interest are highlighted in grey and underlined. FIG. 3B shows boxplots of transient expression assay results for the transcript leaders of GmPsbS1, GmVDE1, GmZEP3, VuPsbS and VuZEP1. From top to bottom, “GmPsbS” (white) designates a construct containing the wild-type transcript leader of GmPsbS1, “gmpsbs-atg1” (white) designates a construct containing the mutated ATG1-uORF of GmPsbS1, “gmpsbs-ttg6” (white) designates a construct containing the mutated TTG6-uORF of GmPsbS1; “GmVDE” (dense stipples) designates a construct containing the wild-type transcript leader of GmVDE1, “gmvde-atg1” (dense stipples) designates a construct containing the mutated ATG1-uORF of GmVDE1, “gmvde-atg2” (dense stipples) designates a construct containing the mutated ATG2-uORF of GmVDE1, and “gmvde-atg3” (dense stipples) designates a construct containing the mutated ATG3-uORF of GmVDE1; “GmZEP” (light stripes) designates a construct containing the wild-type transcript leader of GmZEP3, “gmzep-ctg1” (light stripes) designates a construct containing the mutated CTG1-uORF of GmZEP3, “gmzep-ctg2” (light stripes) designates a construct containing the mutated CTG2-uORF of GmZEP3, and “gmzep-ttg1” (light stripes) designates a construct containing the mutated TTG1-uORF of GmZEP3; “VuPsbS” (light stippling) designates a construct containing the wild-type transcript leader of VuPsbS, “vupsbs-atg1” (light stippling) designates a construct containing the mutated atg1-uORF of VuPsbS, “vupsbs-ttg1” (light stippling) designates a construct containing the mutated ttg1-uORF of VuPsbS, and “vupsbs-ttg2” (light stippling) designates a construct containing the mutated ttg2-uORF of VuPsbS; and “VuZEP” (bold stripes) designates a construct containing the wild-type transcript leader of VuZEP1, “vuzep-atg1” (bold stripes) designates a construct containing the mutated atg1-uORF of VuZEP1, “vuzep-atg2” (bold stripes) designates a construct containing the mutated atg2-uORF of VuZEP1, and “vuzep-atg3” (bold stripes) designates a construct containing the mutated atg3-uORF of VuZEP1. Relative fluorescence for each construct is reported in a ratio of the Relative Fluorescence Units (RFU) measured in the mNeonGreen vs. the tdTomato channel. The excitation/emission wavelengths are 490 nm/520 nm for mNeonGreen and 540 nm/595 nm for tdTomato respectively. Obtained relative fluorescence values are normalized to the mean values of the wild-type transcript leaders respectively. The mean relative fluorescence of the respective wild-type are highlighted by the dashed line. Values with corresponding brackets at the right of the boxplot indicate the adjusted p-values of comparisons between transcript leader gene with and without uORF disruption. P-values are determined by a nonparametric bootstrap for two-sample t-testing and are adjusted for multiple testing after Benjamini & Hochberg (1995). Adjusted p-values are indicated for mutations resulting in signal increases >40%, n=8-12. FIG. 3C shows a bar plot of average relative quantities of GmPsbS, GmVDE1, GmZEP, and VuPsbS mRNA between tissue transiently expressing the GmPsbS, GmVDE, GmZEP or VuPsbS constructs (WT; constructs containing the functional uORFs) and tissue expressing gmpsbs-atg1, gmvde-atg1, gmzep-ttg1, vupsbs-atg1 and vupsbs-ttg4 constructs (constructs containing the mutated uORFs) plotted along the y-axis. Average relative transcript levels were compared via analysis of variance testing (ANOVA). The sample size for each tested construct was 3-4.

FIG. 4 shows a plasmid 15,702 base pairs (15,702 bp) in length containing the construct designated RC00530, wherein there are two single guide RNAs (sgRNAs) flanking each uORF for a total of 6 gRNAs, designed to confer a triple uORF knockout (“KO”) of genes GmVDE, GmPsbS1, and GmZEP (referred to in combination as “GmVPZ”). The plasmid includes part of publicly available direct cloning plasmid “pDIRECT_23C” (Cermak et al. 2017. A Multipurpose Toolkit to Enable Advanced Genome Engineering in Plants. Plant Cell. 29(6):1196-1217). Included plasmid components are as follows: “KanR”=Kanamycin resistance 2 gene sequence; “T-DNA border (L)”=the left border of the T-DNA sequence; “35S terminator”=a terminator sequence originally from the Cauliflower Mosaic Virus (CaMV); “SV40 NLS (with linker)”=simian virus 40 nuclear localization signals with a linker; “CmYLCV Promoter”=the promoter from the Cestrum yellow leaf curling virus; “Cys4”=Csy4 endoribonuclease from Pseudomonas aeruginosa; “gRNA repeat”=direct repeats used in gRNA design; a DraIII cutting site is included; “rep origin”=replication of origin; “BsaI site removed (T>C)”=location of mutation from T to C of a nucleotide for the coding of BsaI restriction endonuclease originally found in Bacillus stearothermophilus 6-55 (Z. Chen); “SapI site removed (G>C)”=location of mutation from G to C of a nucleotide for the coding of SapI restriction endonuclease originally found in Saccharopolyspora species (D. Comb); “bom site from pBR322”=basis of mobility region from plasmid BR322 (Boyer 1977); “bar (phosphinothricinR)”=the Bialaphos resistance (BAR) gene that inactivates glufosinate (also known as phosphinothricin); “P2A”=2A self-cleaving peptide from porcine teschovirus-1; “D10”=Aspartic acid 10, a catalytic residue in the RuvC domain; “H840”=Histidine 840, a catalytic residue in the HNH domain; “AtCas9”=Cas9 endonuclease from Arabidopsis thaliana; and “SpCas9_AtCO”=Cas9 isolated from Streptococcus pyogenes, codon-optimized for expression in A. thaliana.

FIG. 5 shows a plasmid 15,702 base pairs (15,702 bp) in length containing the construct designated RC00531, wherein there are three single guide RNAs (sgRNAs) flanking each uORF for a total of 6 gRNAs, designed to confer a double uORF knockout (“KO”) of genes GmVDE and GmZEP (referred to in combination as “GmVZ”). The plasmid includes part of publicly available direct cloning plasmid “pDIRECT_23C” (Cermak et al. 2017. A Multipurpose Toolkit to Enable Advanced Genome Engineering in Plants. Plant Cell. 29(6):1196-1217). Included plasmid components are as follows: “KanR”=Kanamycin resistance 2 gene; “P2A”=2A self-cleaving peptide from porcine teschovirus-1; “T-DNA border (L)”=the left border of the T-DNA sequence; “35S terminator”=a terminator sequence originally from the Cauliflower Mosaic Virus (CaMV); a DraIII cut site is included; “SV40 NLS (with linker)”=simian virus 40 nuclear localization signals with a linker; “CmYLCV Promoter”=the promoter from the Cestrum yellow leaf curling virus; “Cys4”=Csy4 endoribonuclease from Pseudomonas aeruginosa; “gRNA repeat”=direct repeats used in gRNA design; “rep origin”=replication of origin; “BsaI site removed (T>C)”=location of mutation from T to C of a nucleotide for the coding of BsaI restriction endonuclease originally found in Bacillus stearothermophilus 6-55 (Z. Chen); “SapI site removed (G>C)”=location of mutation from G to C of a nucleotide for the coding of SapI restriction endonuclease originally found in Saccharopolyspora species (D. Comb); “bom site from pBR322”=basis of mobility region from plasmid BR322 (Boyer 1977); “bar (phosphinothricinR)”=the Bialaphos resistance (BAR) gene that inactivates glufosinate (also known as phosphinothricin); “P2A”=2A self-cleaving peptide from porcine teschovirus-1; “D10”=Aspartic acid 10, a catalytic residue in the RuvC domain; “H840”=Histidine 840, a catalytic residue in the RuvC domain; “AtCas9”=Cas9 endonuclease from Arabidopsis thaliana; and “SpCas9_AtCO”=Cas9 isolated from Streptococcus pyogenes, codon-optimized for expression in A. thaliana.

### DETAILED DESCRIPTION

The following description sets forth exemplary methods, parameters, and the like. It should be recognized, however, that such description is not intended as a limitation on the scope of the present disclosure but is instead provided as a description of exemplary embodiments.

### Genetically Modified Plants and Related Methods

An aspect of the disclosure includes a genetically modified plant including one or more edited endogenous nucleotide sequences encoding a photosystem II subunit S (PsbS) polypeptide, a zeaxanthin epoxidase (ZEP) polypeptide, or a violaxanthin de-epoxidase (VDE) polypeptide, wherein the one or more edited nucleotide sequences are edited at one or more upstream open reading frames (uORFs) in a transcript leader sequence (TLS). In a further embodiment of this aspect, the edits at one or more uORFs increase expression of the downstream main open reading frame (mORF) encoding the PsbS polypeptide, the ZEP polypeptide, and/or the VDE polypeptide. In a further embodiment of this aspect, which may be combined with any of the preceding embodiments, the edited endogenous nucleotide sequences were edited at all uORFs in a TLS. In an additional embodiment of this aspect, which may be combined with any of the preceding embodiments, the edited endogenous nucleotide sequences were edited at two or more, three or more, four or more, five or more, six or more, seven or more, eight or more, or nine uORFs in a TLS. In still another embodiment of this aspect, which may be combined with any of the preceding embodiments, (i) the editing removed uORF repression, increased efficiency of mORF translation re-initiation, removed stalling of a ribosome, removed inhibition of translation of downstream ORFs, or increased translational efficiency of downstream ORFs; (ii) the editing resulted in disruption of an uORF start codon, and wherein disruption was achieved by replacement of the uORF start codon with a nucleotide triplet, deletion of the third base of the uORF start codon, a frameshift mutation to disrupt an uORF reading frame, insertion of one or more nucleotides in the uORF, or deletion of one or more nucleotides in the uORF; (iii) uORF repression is reduced or removed, efficiency of mORF translation re-initiation is increased, stalling of a ribosome is reduced or removed, inhibition of translation of downstream ORFs is reduced or removed, or translational efficiency of downstream ORFs is increased; (iv) an uORF start codon is disrupted; and/or (v) the uORF start codon was replaced with a nucleotide triplet, deletion of the third base of the uORF start codon, a frameshift mutation to disrupt an uORF reading frame, insertion of one or more nucleotides in the uORF, or deletion of one or more nucleotides in the uORF. The nucleotide triplet may be AAA, TTT, CCC, or GGG. In another embodiment of this aspect, which may be combined with any of the preceding embodiments, edited endogenous nucleotide sequences encoding the PsbS polypeptide include SEQ ID NO: 2 (mutated TLS used in gmpsbs-atg1 construct), SEQ ID NO: 3 (mutated TLS used in gmpsbs-ttg6 construct), SEQ ID NO: 13 (mutated TLS used in vupsbs-atg4 construct), SEQ ID NO: 14 (mutated TLS used in vupsbs-ttg3 construct), and SEQ ID NO: 15 (mutated TLS used in vupsbs-ttg4 construct). In an additional embodiment of this aspect, the edited endogenous nucleotide sequences encoding the PsbS polypeptide include SEQ ID NO: 2 (mutated TLS used in vupsbs-atg4 construct) or SEQ ID NO: 15 (mutated TLS used in vupsbs-ttg4 construct). In a further embodiment of this aspect, which may be combined with any of the preceding embodiments, edited endogenous nucleotide sequences encoding the ZEP polypeptide include SEQ ID NO: 9 (mutated TLS used in gmzep-ctg1 construct), SEQ ID NO: 10 (mutated TLS used in gmzep-ctg2 construct), SEQ ID NO: 11 (mutated TLS used in gmzep-ttg1 construct), SEQ ID NO: 17 (mutated TLS used in vuzep-atg1 construct), SEQ ID NO: 18 (mutated TLS used in vuzep-atg2 construct), and SEQ ID NO: 19 (mutated TLS used in vuzep-atg3 construct). In an additional embodiment of this aspect, the edited endogenous nucleotide sequences encoding the ZEP polypeptide include SEQ ID NO: 11 (mutated TLS used in gmzep-ttg1 construct). In yet another embodiment of this aspect, which may be combined with any of the preceding embodiments, edited endogenous nucleotide sequences encoding the VDE polypeptide include SEQ ID NO: 5 (mutated TLS used in gmvde-atg1 construct), SEQ ID NO: 6 (mutated TLS used in gmvde-atg2 construct), and SEQ ID NO: 7 (mutated TLS used in gmvde-atg3 construct). In an additional embodiment of this aspect, the edited endogenous nucleotide sequences encoding the VDE polypeptide include SEQ ID NO: 5 (mutated TLS used in gmvde-atg1 construct). In yet another embodiment of this aspect, which may be combined with any of the preceding embodiments, the genetically modified plant includes edited endogenous nucleotide sequences encoding the ZEP polypeptide and the VDE polypeptide or the PsbS polypeptide and the ZEP polypeptide. An additional embodiment of this aspect, which may be combined with any of the preceding embodiments, includes edited endogenous nucleotide sequences encoding the PsbS polypeptide, the ZEP polypeptide, and the VDE polypeptide.

Still another embodiment of this aspect, which may be combined with any of the preceding embodiments, includes further genetic modifications that increase expression of one or more of the PsbS polypeptide, the ZEP polypeptide, or the VDE polypeptide; wherein the expression of the PsbS polypeptide is increased by expressing a transfected nucleotide sequence encoding the PsbS polypeptide in the genetically modified plant, by a genetic modification in a promoter of the edited endogenous nucleotide sequence encoding the PsbS polypeptide and/or by a genetic modification in a promoter of an endogenous nucleotide sequence encoding the PsbS polypeptide; wherein the expression of the ZEP polypeptide is increased by expressing a transfected nucleotide sequence encoding the ZEP polypeptide in the genetically modified plant, by a genetic modification in a promoter of the edited endogenous nucleotide sequence encoding the ZEP polypeptide, and/or by a genetic modification in a promoter of an endogenous nucleotide sequence encoding the ZEP polypeptide; and/or wherein the expression of the VDE polypeptide is increased by expressing a transfected nucleotide sequence encoding the VDE polypeptide in the genetically modified plant, by a genetic modification in a promoter of the edited endogenous nucleotide sequence encoding the VDE polypeptide, and/or by a genetic modification in a promoter of an endogenous nucleotide sequence encoding the VDE polypeptide. In yet another embodiment of this aspect, the expression of the PsbS polypeptide is increased by expressing the transfected nucleotide sequence encoding the PsbS polypeptide, and wherein the transfected nucleotide sequence is operably linked to at least one expression control sequence; wherein the expression of the VDE polypeptide is increased by expressing the transfected nucleotide sequence encoding the VDE polypeptide, and wherein the transfected nucleotide sequence is operably linked to at least one expression control sequence; and/or wherein the expression of the ZEP polypeptide is increased by expressing the transfected nucleotide sequence encoding the ZEP polypeptide, and wherein the transfected nucleotide sequence is operably linked to at least one expression control sequence. In still another embodiment of this aspect, which may be combined with any of the preceding embodiments that has increased expression by expressing a transfected nucleotide sequence and/or by a genetic modification in a promoter of a nucleotide sequence, the one or more edited endogenous nucleotide sequences encodes the ZEP polypeptide, and wherein the expression of the VDE polypeptide is increased by expressing the transfected nucleotide sequence encoding the VDE polypeptide; wherein the one or more edited endogenous nucleotide sequences encodes the VDE polypeptide, and wherein the expression of the ZEP polypeptide is increased by expressing the transfected nucleotide sequence encoding the ZEP polypeptide; wherein the one or more edited endogenous nucleotide sequences encodes the PsbS polypeptide, and wherein the expression of the ZEP polypeptide is increased by expressing the transfected nucleotide sequence encoding the ZEP polypeptide; wherein the one or more edited endogenous nucleotide sequences encodes the ZEP polypeptide, and wherein the expression of the PsbS polypeptide is increased by expressing the transfected nucleotide sequence encoding the PsbS polypeptide. In a further embodiment of this aspect, which may be combined with any of the preceding embodiments that has the edits at one or more uORFs increasing expression of the downstream mORF encoding the PsbS polypeptide, the ZEP polypeptide, and/or the VDE polypeptide, the expression of both the ZEP polypeptide and the VDE polypeptide are increased under the same conditions as compared to a control plant or the expression of both the PsbS polypeptide and the ZEP polypeptide are increased as compared to a control plant grown under the same conditions. In yet another embodiment of this aspect, which may be combined with any of the preceding embodiments that has the edits at one or more uORFs increasing expression of the downstream mORF encoding the PsbS polypeptide, the ZEP polypeptide, and/or the VDE polypeptide, the expression of all three of the ZEP polypeptide, the PsbS polypeptide, and the VDE polypeptide are increased as compared to a control plant grown under the same conditions. In still another embodiment of this aspect, which may be combined with any of the preceding embodiments that has increased expression of two or more of the ZEP polypeptide, the PsbS polypeptide, and the VDE polypeptide under the same conditions, the conditions are fluctuating light conditions. In an additional embodiment of this aspect, which may be combined with any of the preceding embodiments, the transfected nucleotide sequence was introduced with an expression vector having one or more heterologous nucleotide sequences that encode PsbS, ZEP and/or VDE. In yet another embodiment of this aspect, the vector contained a promoter of Rbcs1A, GAPA-1 or FBA2. In an additional embodiment of this aspect, the Rbcs1A promoter drove expression of ZEP, a GAPA-1 promoter drove expression of PsbS, and an FBA2 promoter drove expression of VDE. In yet another embodiment of this aspect, the vector was a T-DNA. In a further embodiment of this aspect, the vector had a nucleotide sequence encoding a polypeptide that provided antibiotic resistance. In still another embodiment of this aspect, the vector had a left border (LB) and right border (RB) domain flanking the expression control sequences and the nucleotide sequence encoding the PsbS, ZEP and VDE polypeptides. In a further embodiment of this aspect, the genetic modification in the promoter of the nucleotide sequence was achieved by a genome editing system. In some embodiments, the genome editing system was CRISPR. In an additional embodiment of this aspect, which may be combined with any of the preceding embodiments, the transcript level of the nucleotide sequence encoding the VDE polypeptide is increased 3-fold as compared to the control plant, and wherein the transcript level of the nucleotide sequence encoding the ZEP polypeptide is increased 8-fold as compared to the control plant; the transcript level of the nucleotide sequence encoding the VDE polypeptide is increased 10-fold as compared to the control plant, and wherein the transcript level of the nucleotide sequence encoding the ZEP polypeptide is increased 6-fold as compared to the control plant; the transcript level of the nucleotide sequence encoding the VDE polypeptide is increased 4-fold as compared to the control plant, and wherein the transcript level of the nucleotide sequence encoding the ZEP polypeptide is increased 7-fold as cornpared to the control plant; the protein level of the VDE polypeptide is increased 16-fold as compared to the control plant, and wherein the protein level of the ZEP polypeptide is increased 80-fold as compared to the control plant; the protein level of the VDE polypeptide is increased 30-fold as compared to the control plant, and wherein the protein level of the ZEP polypeptide is increased 74-fold as compared to the control plant; the protein level of the VDE polypeptide is increased 47-fold as compared to the control plant, and wherein the protein level of the ZEP polypeptide is increased 75-fold as compared to the control plant; the increase of transcript level as compared to the control plant between the VDE polypeptide and the ZEP polypeptide has a ratio selected from the group consisting of 3:8, 10:6, and 4:7; the increase of protein level as compared to the control plant between the VDE polypeptide and the ZEP polypeptide has a ratio selected from the group consisting of 16:80, 30:74, and 47:75; the increase of transcript level of the VDE polypeptide as compared to the control plant is from about 3-fold to about 10-fold, and wherein the increase of transcript level of the ZEP polypeptide as compared to the control plant is from about 6-fold to about 8-fold; or the increase of protein level of the VDE polypeptide as compared to the control plant is in from about 16-fold to about 47-fold, and wherein the increase of protein level of the ZEP polypeptide as compared to the control plant is from about 74-fold to about 80-fold. PsbS homolog sequences for use in any of the above embodiments include the nucleotide sequence SEQ ID NO: 27, and nucleotide sequences encoding any of the polypeptide sequences SEQ ID NO: 30, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, or SEQ ID NO: 51. VDE homolog sequences for use in any of the above embodiments include the nucleotide sequence SEQ ID NO: 29, and nucleotide sequences encoding any of the polypeptide sequences SEQ ID NO: 32, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, or SEQ ID NO: 88. ZEP homolog sequences for use in any of the above embodiments include the nucleotide sequence SEQ ID NO: 28, and nucleotide sequences encoding any of the polypeptide sequences SEQ ID NO: 31, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, or SEQ ID NO: 70.

In a further embodiment of this aspect, which may be combined with any of the preceding embodiments, the editing was done using a gene editing technique selected from a transcription activator-like effector nuclease (TALEN) gene editing technique, a clustered Regularly Interspaced Short Palindromic Repeat (CRISPR/Cas) gene editing technique, a zinc-finger nuclease (ZFN) gene editing technique, or combinations of the foregoing. In an additional embodiment of this aspect, the gene editing technique was a clustered Regularly Interspaced Short Palindromic Repeat (CRISPR/Cas) gene editing technique, and wherein the Cas enzymes were selected from the group of Cas9, Cas12, Cas12a, Cas13, Cas14, CasX, or CasY. In some embodiments, the editing was introduced with targeted edits (e.g., with prime editing or base editing), which may be more effective than, e.g., Cas9-mediated editing.

Other methods of modulating, downregulating, or knocking down/knocking out uORFs known in the art can be used. For example, introducing additions, deletions, substitutions, mutations, modifications of start codons, or stop codons can be used. Modifications can be made at regions in the uORF associated with expression modulation. RNA can be edited by a molecular process through which the nucleotide sequence of an RNA molecule is altered post-transcriptionally. For example, ADAR (Adenosine Deaminase Acting on RNA), CRISPR/Cas systems adapted to perform targeted RNA editing without altering the DNA itself, or antisense oligonucleotides can be used to direct the editing enzymes to specific RNA sequences.

In still another embodiment of this aspect, which may be combined with any of the preceding embodiments, the plant is a crop plant, a model plant, a monocotyledonous plant, a dicotyledonous plant, a plant with Crassulacean acid metabolism (CAM) photosynthesis, a plant with C3 photosynthesis, a plant with C4 photosynthesis, an annual plant, a greenhouse plant, a horticultural flowering plant, a perennial plant, a switchgrass plant, a maize plant, a biomass plant, an Arabidopsis thaliana plant, a tobacco (Nicotiana tabacum) plant, a rice (Oryza sativa) plant, a corn (Zea mays) plant, a sorghum (Sorghum bicolor) (sweet sorghum or grain sorghum) plant, a soybean (Glycine max) plant, a cowpea (Vigna unguiculata) plant, a poplar (Populus spp.) plant, a eucalyptus (Eucalyptus spp.) plant, a cassava (Manihot esculenta) plant, a barley (Hordeum vulgare) plant, a potato (Solanum tuberosum) plant, a sugarcane (Saccharum spp.) plant, an alfalfa (Medicago sativa) plant, a Miscanthus plant, an energy cane plant, an elephant grass plant, a wheat plant, an oat plant, an oil palm plant, a safflower plant, a sesame plant, a flax plant, a cotton plant, a sunflower plant, a Camelina plant, a Brassica napus plant, a Brassica carinata plant, a Brassica juncea plant, a pearl millet plant, a foxtail millet plant, an other grain plant, an oilseed plant, a vegetable crop plant, a forage crop plant, an industrial crop plant, or a woody crop plant. In a further embodiment of this aspect, the plant is a soybean (Glycine max) plant or a cowpea (Vigna unguiculata) plant. In yet another embodiment of this aspect, which may be combined with any of the preceding embodiments, the plant has improved growth under fluctuation light conditions as compared to the control plant grown under the same fluctuating light conditions; the plant has increased lutein under fluctuating light conditions as compared to the control plant grown under the same fluctuating light conditions; the plant has increased photosynthetic efficiency under fluctuating light conditions as compared to the control plant grown under the same fluctuating light conditions; the plant has improved photoprotection efficiency under fluctuating light conditions as compared to the control plant grown under the same fluctuating light conditions; the plant has an increased rate of relaxation of non-photochemical quenching (NPQ) under fluctuating light conditions as compared to the control plant grown under the same fluctuating light conditions; and/or the plant has improved quantum yield and CO₂ fixation under fluctuating light conditions as compared to the control plant grown under the same fluctuating light conditions.

Some aspects of the present disclosure relate to a genetically modified plant including one or more edited uORFs of an endogenous PsbS gene, one or more edited uORFs of an endogenous ZEP gene, one or more edited uORFs of an endogenous VDE gene, or a combination thereof. In a further embodiment of this aspect, the one or more edited uORFs increase expression of the downstream mORF of the endogenous PsbS gene, the endogenous ZEP gene, and/or the endogenous VDE gene. In a further embodiment of this aspect, which may be combined with any of the preceding embodiments, the edited endogenous nucleotide sequences were edited at all uORFs in a transcript leader sequence (TLS). In an additional embodiment of this aspect, which may be combined with any of the preceding embodiments, the edited endogenous nucleotide sequences were edited at two or more, three or more, four or more, five or more, six or more, seven or more, eight or more, or nine uORFs in a TLS. In still another embodiment of this aspect, which may be combined with any of the preceding embodiments, (i) the editing removed uORF repression, increased efficiency of mORF translation re-initiation, removed stalling of a ribosome, removed inhibition of translation of downstream ORFs, or increased translational efficiency of downstream ORFs; (ii) the editing resulted in disruption of an uORF start codon, and wherein disruption was achieved by replacement of the uORF start codon with a nucleotide triplet, deletion of the third base of the uORF start codon, a frameshift mutation to disrupt an uORF reading frame, insertion of one or more nucleotides in the uORF, or deletion of one or more nucleotides in the uORF; (iii) uORF repression is reduced or removed, efficiency of mORF translation re-initiation is increased, stalling of a ribosome is reduced or removed, inhibition of translation of downstream ORFs is reduced or removed, or translational efficiency of downstream ORFs is increased; (iv) an uORF start codon is disrupted; and/or (v) the uORF start codon was replaced with a nucleotide triplet, deletion of the third base of the uORF start codon, a frameshift mutation to disrupt an uORF reading frame, insertion of one or more nucleotides in the uORF, or deletion of one or more nucleotides in the uORF. The nucleotide triplet may be AAA, TTT, CCC, or GGG. In another embodiment of this aspect, which may be combined with any of the preceding embodiments, edited endogenous nucleotide sequences encoding the PsbS polypeptide include SEQ ID NO: 2 (mutated TLS used in gmpsbs-atg1 construct), SEQ ID NO: 3 (mutated TLS used in gmpsbs-ttg6 construct), SEQ ID NO: 13 (mutated TLS used in vupsbs-atg4 construct), SEQ ID NO: 14 (mutated TLS used in vupsbs-ttg3 construct), and SEQ ID NO: 15 (mutated TLS used in vupsbs-ttg4 construct). In an additional embodiment of this aspect, the edited endogenous nucleotide sequences encoding the PsbS polypeptide include SEQ ID NO: 2 (mutated TLS used in vupsbs-atg4 construct) or SEQ ID NO: 15 (mutated TLS used in vupsbs-ttg4 construct). In a further embodiment of this aspect, which may be combined with any of the preceding embodiments, edited endogenous nucleotide sequences encoding the ZEP polypeptide include SEQ ID NO: 9 (mutated TLS used in gmzep-ctg1 construct), SEQ ID NO: 10 (mutated TLS used in gmzep-ctg2 construct), SEQ ID NO: 11 (mutated TLS used in gmzep-ttg1 construct), SEQ ID NO: 17 (mutated TLS used in vuzep-atg1 construct), SEQ ID NO: 18 (mutated TLS used in vuzep-atg2 construct), and SEQ ID NO: 19 (mutated TLS used in vuzep-atg3 construct). In an additional embodiment of this aspect, the edited endogenous nucleotide sequences encoding the ZEP polypeptide include SEQ ID NO: 11 (mutated TLS used in gmzep-ttg1 construct). In yet another embodiment of this aspect, which may be combined with any of the preceding embodiments, edited endogenous nucleotide sequences encoding the VDE polypeptide include SEQ ID NO: 5 (mutated TLS used in gmvde-atg1 construct), SEQ ID NO: 6 (mutated TLS used in gmvde-atg2 construct), and SEQ ID NO: 7 (mutated TLS used in gmvde-atg3 construct). In an additional embodiment of this aspect, the edited endogenous nucleotide sequences encoding the VDE polypeptide include SEQ ID NO: 5 (mutated TLS used in gmvde-atg1 construct).

An additional embodiment of this aspect, which may be combined with any of the preceding embodiments, includes further genetic modifications that increase expression of one or more of the PsbS polypeptide, the ZEP polypeptide, or the VDE polypeptide; wherein the expression of the PsbS polypeptide is increased by expressing a transfected nucleotide sequence encoding the PsbS polypeptide in the genetically modified plant, by a genetic modification in a promoter of the edited endogenous nucleotide sequence encoding the PsbS polypeptide and/or by a genetic modification in a promoter of an endogenous nucleotide sequence encoding the PsbS polypeptide; wherein the expression of the ZEP polypeptide is increased by expressing a transfected nucleotide sequence encoding the ZEP polypeptide in the genetically modified plant, by a genetic modification in a promoter of the edited endogenous nucleotide sequence encoding the ZEP polypeptide, and/or by a genetic modification in a promoter of an endogenous nucleotide sequence encoding the ZEP polypeptide; and/or wherein the expression of the VDE polypeptide is increased by expressing a transfected nucleotide sequence encoding the VDE polypeptide in the genetically modified plant, by a genetic modification in a promoter of the edited endogenous nucleotide sequence encoding the VDE polypeptide, and/or by a genetic modification in a promoter of an endogenous nucleotide sequence encoding the VDE polypeptide. In an additional embodiment of this aspect, the expression of the PsbS polypeptide is increased by expressing the transfected nucleotide sequence encoding the PsbS polypeptide, and wherein the transfected nucleotide sequence is operably linked to at least one expression control sequence; wherein the expression of the VDE polypeptide is increased by expressing the transfected nucleotide sequence encoding the VDE polypeptide, and wherein the transfected nucleotide sequence is operably linked to at least one expression control sequence; and/or wherein the expression of the ZEP polypeptide is increased by expressing the transfected nucleotide sequence encoding the ZEP polypeptide, and wherein the transfected nucleotide sequence is operably linked to at least one expression control sequence. In yet another embodiment of this aspect, which may be combined with any of the preceding embodiments that has the one or more edited uORFs increasing expression of the downstream mORF of the endogenous PsbS gene, the endogenous ZEP gene, and/or the endogenous VDE gene, the expression of both the ZEP polypeptide and the VDE polypeptide are increased under the same conditions as compared to a control plant or the expression of both the PsbS polypeptide and the ZEP polypeptide are increased as compared to a control plant grown under the same conditions. In a further embodiment of this aspect, which may be combined with any of the preceding embodiments that has the one or more edited uORFs increasing expression of the downstream mORF of the endogenous PsbS gene, the expression of all three of the ZEP polypeptide, the PsbS polypeptide, and the VDE polypeptide are increased as compared to a control plant grown under the same conditions. In still another embodiment of this aspect, which may be combined with any of the preceding embodiments that has increased expression of two or more of the ZEP polypeptide, the PsbS polypeptide, and the VDE polypeptide under the same conditions, the conditions are fluctuating light conditions. In an additional embodiment of this aspect, which may be combined with any of the preceding embodiments, the transfected nucleotide sequence was introduced with an expression vector having one or more heterologous nucleotide sequences that encode PsbS, ZEP and/or VDE. In yet another embodiment of this aspect, the vector contained a promoter of Rbcs1A, GAPA-1 or FBA2. In an additional embodiment of this aspect, the Rbcs1A promoter drove expression of ZEP, a GAPA-1 promoter drove expression of PsbS, and an FBA2 promoter drove expression of VDE. In yet another embodiment of this aspect, the vector was a T-DNA. In a further embodiment of this aspect, the vector had a nucleotide sequence encoding a polypeptide that provided antibiotic resistance. In still another embodiment of this aspect, the vector had a left border (LB) and right border (RB) domain flanking the expression control sequences and the nucleotide sequence encoding the PsbS, ZEP and VDE polypeptides. In a further embodiment of this aspect, the genetic modification in the promoter of the nucleotide sequence was achieved by a genome editing system. In some embodiments, the genome editing system was CRISPR. In an additional embodiment of this aspect, which may be combined with any of the preceding embodiments, the transcript level of the nucleotide sequence encoding the VDE polypeptide is increased 3-fold as compared to the control plant, and wherein the transcript level of the nucleotide sequence encoding the ZEP polypeptide is increased 8-fold as compared to the control plant; the transcript level of the nucleotide sequence encoding the VDE polypeptide is increased 10-fold as compared to the control plant, and wherein the transcript level of the nucleotide sequence encoding the ZEP polypeptide is increased 6-fold as compared to the control plant; the transcript level of the nucleotide sequence encoding the VDE polypeptide is increased 4-fold as compared to the control plant, and wherein the transcript level of the nucleotide sequence encoding the ZEP polypeptide is increased 7-fold as cornpared to the control plant; the protein level of the VDE polypeptide is increased 16-fold as compared to the control plant, and wherein the protein level of the ZEP polypeptide is increased 80-fold as compared to the control plant; the protein level of the VDE polypeptide is increased 30-fold as compared to the control plant, and wherein the protein level of the ZEP polypeptide is increased 74-fold as compared to the control plant; the protein level of the VDE polypeptide is increased 47-fold as compared to the control plant, and wherein the protein level of the ZEP polypeptide is increased 75-fold as compared to the control plant; the increase of transcript level as compared to the control plant between the VDE polypeptide and the ZEP polypeptide has a ratio selected from the group consisting of 3:8, 10:6, and 4:7; the increase of protein level as compared to the control plant between the VDE polypeptide and the ZEP polypeptide has a ratio selected from the group consisting of 16:80, 30:74, and 47:75; the increase of transcript level of the VDE polypeptide as compared to the control plant is from about 3-fold to about 10-fold, and wherein the increase of transcript level of the ZEP polypeptide as compared to the control plant is from about 6-fold to about 8-fold; or the increase of protein level of the VDE polypeptide as compared to the control plant is in from about 16-fold to about 47-fold, and wherein the increase of protein level of the ZEP polypeptide as compared to the control plant is from about 74-fold to about 80-fold. PsbS homolog sequences for use in any of the above embodiments include the nucleotide sequence SEQ ID NO: 27, and nucleotide sequences encoding any of the polypeptide sequences SEQ ID NO: 30, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, or SEQ ID NO: 51. VDE homolog sequences for use in any of the above embodiments include the nucleotide sequence SEQ ID NO: 29, and nucleotide sequences encoding any of the polypeptide sequences SEQ ID NO: 32, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, or SEQ ID NO: 88. ZEP homolog sequences for use in any of the above embodiments include the nucleotide sequence SEQ ID NO: 28, and nucleotide sequences encoding any of the polypeptide sequences SEQ ID NO: 31, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, or SEQ ID NO: 70. In still another embodiment of this aspect, which may be combined with any of the preceding embodiments, the plant has improved growth under fluctuation light conditions as compared to a control plant grown under the same fluctuating light conditions; the plant has increased lutein under fluctuating light conditions as compared to a control plant grown under the same fluctuating light conditions; the plant has increased photosynthetic efficiency under fluctuating light conditions as compared to a control plant grown under the same fluctuating light conditions; the plant has improved photoprotection efficiency under fluctuating light conditions as compared to a control plant grown under the same fluctuating light conditions; the plant has an increased rate of relaxation of non-photochemical quenching (NPQ) under fluctuating light conditions as compared to a control plant grown under the same fluctuating light conditions; and/or the plant has improved quantum yield and CO₂ fixation under fluctuating light conditions as compared to a control plant grown under the same fluctuating light conditions. In yet another embodiment of this aspect, which may be combined with any of the preceding embodiments, the plant is a soybean (Glycine max) plant or a cowpea (Vigna unguiculata) plant.

Further aspects of the disclosure include a plant part, plant cell, or seed of the genetically modified plant of any one of the preceding embodiments.

An additional aspect of the disclosure includes methods of producing the genetically modified plant of any one of the preceding embodiments, including: (a) providing a plant, plant part, plant cell, tissue, or other explant including an endogenous PsbS gene, an endogenous ZEP gene, and/or an endogenous VDE gene; (b) selecting one or more uORFs in a TLS of the endogenous PsbS gene, the endogenous ZEP gene, and/or the endogenous VDE gene for editing; and (c) using a gene editing technique to edit the one or more uORFs of the endogenous PsbS gene, the endogenous ZEP gene, and/or the endogenous VDE gene in the plant, plant part, plant cell, tissue, or other explant, and optionally regenerating the plant cell, tissue, or other explant into a genetically altered plantlet that is grown into a plant, to produce a genetically modified plant including one or more edited PsbS, ZEP, and/or VDE genes. In another embodiment of this aspect, the edits at one or more uORFs increase expression of the downstream mORF of the endogenous PsbS gene, the endogenous ZEP gene, and/or the endogenous VDE gene. In still another embodiment of this aspect, which may be combined with any of the preceding embodiments, the edited endogenous genes are edited at all uORFs in a TLS. In a further embodiment of this aspect, which may be combined with any of the preceding embodiments, the edited endogenous genes are edited at two or more, three or more, four or more, five or more, six or more, seven or more, eight or more, or nine uORFs in a TLS. In a further embodiment of this aspect, the editing removes uORF repression, increases efficiency of mORF translation re-initiation, removes stalling of a ribosome, removes inhibition of translation of downstream ORFs, or increases translational efficiency of downstream ORFs. In an additional embodiment of this aspect, which may be combined with any of the preceding embodiments, the editing results in disruption of an uORF start codon, and wherein disruption is achieved by replacement of the uORF start codon with a nucleotide triplet, deletion of the third base of the uORF start codon, a frameshift mutation to disrupt a uORF reading frame, insertion of one or more nucleotides, or deletion of one or more nucleotides. The nucleotide triplet may be AAA, TTT, CCC, or GGG. In another embodiment of this aspect, which may be combined with any of the preceding embodiments, edited endogenous nucleotide sequences encoding the PsbS polypeptide include SEQ ID NO: 2 (mutated TLS used in gmpsbs-atg1 construct), SEQ ID NO: 3 (mutated TLS used in gmpsbs-ttg6 construct), SEQ ID NO: 13 (mutated TLS used in vupsbs-atg4 construct), SEQ ID NO: 14 (mutated TLS used in vupsbs-ttg3 construct), and SEQ ID NO: 15 (mutated TLS used in vupsbs-ttg4 construct). In an additional embodiment of this aspect, the edited endogenous nucleotide sequences encoding the PsbS polypeptide include SEQ ID NO: 2 (mutated TLS used in vupsbs-atg4 construct) or SEQ ID NO: 15 (mutated TLS used in vupsbs-ttg4 construct). In a further embodiment of this aspect, which may be combined with any of the preceding embodiments, edited endogenous nucleotide sequences encoding the ZEP polypeptide include SEQ ID NO: 9 (mutated TLS used in gmzep-ctg1 construct), SEQ ID NO: 10 (mutated TLS used in gmzep-ctg2 construct), SEQ ID NO: 11 (mutated TLS used in gmzep-ttg1 construct), SEQ ID NO: 17 (mutated TLS used in vuzep-atg1 construct), SEQ ID NO: 18 (mutated TLS used in vuzep-atg2 construct), and SEQ ID NO: 19 (mutated TLS used in vuzep-atg3 construct). In an additional embodiment of this aspect, the edited endogenous nucleotide sequences encoding the ZEP polypeptide include SEQ ID NO: 11 (mutated TLS used in gmzep-ttg1 construct). In yet another embodiment of this aspect, which may be combined with any of the preceding embodiments, edited endogenous nucleotide sequences encoding the VDE polypeptide include SEQ ID NO: 5 (mutated TLS used in gmvde-atg1 construct), SEQ ID NO: 6 (mutated TLS used in gmvde-atg2 construct), and SEQ ID NO: 7 (mutated TLS used in gmvde-atg3 construct). In an additional embodiment of this aspect, the edited endogenous nucleotide sequences encoding the VDE polypeptide include SEQ ID NO: 5 (mutated TLS used in gmvde-atg1 construct).

Yet another embodiment of this aspect, which may be combined with any of the preceding embodiments, optionally further includes increasing expression of one or more of the PsbS polypeptide, the VDE polypeptide, or the ZEP polypeptide before step (c) or in step (c), wherein the expression of the PsbS polypeptide is increased by expressing a transfected nucleotide sequence encoding the PsbS polypeptide in the genetically modified plant, by a genetic modification in a promoter of the edited endogenous nucleotide sequence encoding the PsbS polypeptide and/or by a genetic modification in a promoter of an endogenous nucleotide sequence encoding the PsbS polypeptide; wherein the expression of the ZEP polypeptide is increased by expressing a transfected nucleotide sequence encoding the ZEP polypeptide in the genetically modified plant, by a genetic modification in a promoter of the edited endogenous nucleotide sequence encoding the ZEP polypeptide, and/or by a genetic modification in a promoter of an endogenous nucleotide sequence encoding the ZEP polypeptide; and/or wherein the expression of the VDE polypeptide is increased by expressing a transfected nucleotide sequence encoding the VDE polypeptide in the genetically modified plant, by a genetic modification in a promoter of the edited endogenous nucleotide sequence encoding the VDE polypeptide, and/or by a genetic modification in a promoter of an endogenous nucleotide sequence encoding the VDE polypeptide. In a further embodiment of this aspect, the expression of the PsbS polypeptide is increased by expressing the transfected nucleotide sequence encoding the PsbS polypeptide, and wherein the transfected nucleotide sequence is operably linked to at least one expression control sequence; wherein the expression of the VDE polypeptide is increased by expressing the transfected nucleotide sequence encoding the VDE polypeptide, and wherein the transfected nucleotide sequence is operably linked to at least one expression control sequence; and/or wherein the expression of the ZEP polypeptide is increased by expressing the transfected nucleotide sequence encoding the ZEP polypeptide, and wherein the transfected nucleotide sequence is operably linked to at least one expression control sequence. In yet another embodiment of this aspect, which may be combined with any of the preceding embodiments that has the one or more edited uORFs increasing expression of the downstream mORF of the endogenous PsbS gene, the endogenous ZEP gene, and/or the endogenous VDE gene, the expression of both the ZEP polypeptide and the VDE polypeptide are increased under the same conditions as compared to a control plant or the expression of both the PsbS polypeptide and the ZEP polypeptide are increased as compared to a control plant grown under the same conditions. In a further embodiment of this aspect, which may be combined with any of the preceding embodiments that has the one or more edited uORFs increasing expression of the downstream mORF of the endogenous PsbS gene, the expression of all three of the ZEP polypeptide, the PsbS polypeptide, and the VDE polypeptide are increased as compared to a control plant grown under the same conditions. In still another embodiment of this aspect, which may be combined with any of the preceding embodiments that has increased expression of two or more of the ZEP polypeptide, the PsbS polypeptide, and the VDE polypeptide under the same conditions, the conditions are fluctuating light conditions. In an additional embodiment of this aspect, which may be combined with any of the preceding embodiments, the transfected nucleotide sequence was introduced with an expression vector having one or more heterologous nucleotide sequences that encode PsbS, ZEP and/or VDE. In yet another embodiment of this aspect, the vector contained a promoter of Rbcs1A, GAPA-1 or FBA2. In an additional embodiment of this aspect, the Rbcs1A promoter drove expression of ZEP, a GAPA-1 promoter drove expression of PsbS, and an FBA2 promoter drove expression of VDE. In yet another embodiment of this aspect, the vector was a T-DNA. In a further embodiment of this aspect, the vector had a nucleotide sequence encoding a polypeptide that provided antibiotic resistance. In still another embodiment of this aspect, the vector had a left border (LB) and right border (RB) domain flanking the expression control sequences and the nucleotide sequence encoding the PsbS, ZEP and VDE polypeptides. In a further embodiment of this aspect, the genetic modification in the promoter of the nucleotide sequence was achieved by a genome editing system. In some embodiments, the genome editing system was CRISPR. In an additional embodiment of this aspect, which may be combined with any of the preceding embodiments, the transcript level of the nucleotide sequence encoding the VDE polypeptide is increased 3-fold as compared to the control plant, and wherein the transcript level of the nucleotide sequence encoding the ZEP polypeptide is increased 8-fold as compared to the control plant; the transcript level of the nucleotide sequence encoding the VDE polypeptide is increased 10-fold as compared to the control plant, and wherein the transcript level of the nucleotide sequence encoding the ZEP polypeptide is increased 6-fold as compared to the control plant; the transcript level of the nucleotide sequence encoding the VDE polypeptide is increased 4-fold as compared to the control plant, and wherein the transcript level of the nucleotide sequence encoding the ZEP polypeptide is increased 7-fold as compared to the control plant; the protein level of the VDE polypeptide is increased 16-fold as compared to the control plant, and wherein the protein level of the ZEP polypeptide is increased 80-fold as compared to the control plant; the protein level of the VDE polypeptide is increased 30-fold as compared to the control plant, and wherein the protein level of the ZEP polypeptide is increased 74-fold as compared to the control plant; the protein level of the VDE polypeptide is increased 47-fold as compared to the control plant, and wherein the protein level of the ZEP polypeptide is increased 75-fold as compared to the control plant; the increase of transcript level as compared to the control plant between the VDE polypeptide and the ZEP polypeptide has a ratio selected from the group consisting of 3:8, 10:6, and 4:7; the increase of protein level as compared to the control plant between the VDE polypeptide and the ZEP polypeptide has a ratio selected from the group consisting of 16:80, 30:74, and 47:75; the increase of transcript level of the VDE polypeptide as compared to the control plant is from about 3-fold to about 10-fold, and wherein the increase of transcript level of the ZEP polypeptide as compared to the control plant is from about 6-fold to about 8-fold; or the increase of protein level of the VDE polypeptide as compared to the control plant is in from about 16-fold to about 47-fold, and wherein the increase of protein level of the ZEP polypeptide as compared to the control plant is from about 74-fold to about 80-fold. PsbS homolog sequences for use in any of the above embodiments include the nucleotide sequence SEQ ID NO: 27, and nucleotide sequences encoding any of the polypeptide sequences SEQ ID NO: 30, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, or SEQ ID NO: 51. VDE homolog sequences for use in any of the above embodiments include the nucleotide sequence SEQ ID NO: 29, and nucleotide sequences encoding any of the polypeptide sequences SEQ ID NO: 32, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, or SEQ ID NO: 88. ZEP homolog sequences for use in any of the above embodiments include the nucleotide sequence SEQ ID NO: 28, and nucleotide sequences encoding any of the polypeptide sequences SEQ ID NO: 31, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, or SEQ ID NO: 70.

In still another embodiment of this aspect, which may be combined with any of the preceding embodiments, the gene editing technique is a transcription activator-like effector nuclease (TALEN) gene editing technique, a clustered Regularly Interspaced Short Palindromic Repeat (CRISPR/Cas) gene editing technique, a zinc-finger nuclease (ZFN) gene editing technique, or combinations of the foregoing. In a further embodiment of this aspect, the gene editing technique is a clustered Regularly Interspaced Short Palindromic Repeat (CRISPR/Cas) gene editing technique. In yet another embodiment of this aspect, the Cas enzymes are selected from the group of Cas9, Cas12, Cas12a, Cas13, Cas14, CasX, or CasY. In some embodiments, the gene-editing technique is introducing targeted edits (e.g., with prime editing or base editing), which may be more effective than, e.g., Cas9-mediated editing. In yet another embodiment of this aspect, which may be combined with any of the preceding embodiments, the genetically modified plant produced in step (c): has improved growth under fluctuation light conditions as compared to a control plant grown under the same fluctuating light conditions; has increased lutein under fluctuating light conditions as compared to a control plant grown under the same fluctuating light conditions; has increased photosynthetic efficiency under fluctuating light conditions as compared to a control plant grown under the same fluctuating light conditions; has improved photoprotection efficiency under fluctuating light conditions as compared to a control plant grown under the same fluctuating light conditions; has an increased rate of relaxation of non-photochemical quenching (NPQ) under fluctuating light conditions as compared to a control plant grown under the same fluctuating light conditions; and/or has improved quantum yield and CO₂ fixation under fluctuating light conditions as compared to a control plant grown under the same fluctuating light conditions. In still another embodiment of this aspect, which may be combined with any of the preceding embodiments, the plant is a crop plant, a model plant, a monocotyledonous plant, a dicotyledonous plant, a plant with Crassulacean acid metabolism (CAM) photosynthesis, a plant with C3 photosynthesis, a plant with C4 photosynthesis, an annual plant, a greenhouse plant, a horticultural flowering plant, a perennial plant, a switchgrass plant, a maize plant, a biomass plant, an Arabidopsis thaliana plant, a tobacco (Nicotiana tabacum) plant, a rice (Oryza sativa) plant, a corn (Zea mays) plant, a sorghum (Sorghum bicolor) (sweet sorghum or grain sorghum) plant, a soybean (Glycine max) plant, a cowpea (Vigna unguiculata) plant, a poplar (Populus spp.) plant, a eucalyptus (Eucalyptus spp.) plant, a cassava (Manihot esculenta) plant, a barley (Hordeum vulgare) plant, a potato (Solanum tuberosum) plant, a sugarcane (Saccharum spp.) plant, an alfalfa (Medicago sativa) plant, a Miscanthus plant, an energy cane plant, an elephant grass plant, a wheat plant, an oat plant, an oil palm plant, a safflower plant, a sesame plant, a flax plant, a cotton plant, a sunflower plant, a Camelina plant, a Brassica napus plant, a Brassica carinata plant, a Brassica juncea plant, a pearl millet plant, a foxtail millet plant, an other grain plant, an oilseed plant, a vegetable crop plant, a forage crop plant, an industrial crop plant, or a woody crop plant. In a further embodiment of this aspect, the plant is a soybean (Glycine max) plant or a cowpea (Vigna unguiculata) plant.

Further aspects of the present disclosure relate to a genetically altered plant genome including (i) the one or more edited endogenous nucleotide sequences in the genetically modified plant of any one of the preceding embodiments, or (ii) the one or more edited endogenous nucleotide sequences in the genetically modified plant produced by the method of any one of the preceding embodiments.

Additional aspects of the present disclosure relate to a non-regenerable part or cell of the genetically modified plant of any one of the preceding embodiments.

A “control plant” as described herein can be a control sample or a reference sample from a wild-type, an azygous, or a null-segregant plant, species, or sample or from populations thereof. A reference value can be used in place of a control or reference sample, which was previously obtained from a wild-type, azygous, or null-segregant plant, species, or sample or from populations thereof or a group of a wild-type, azygous, or null-segregant plant, species, or sample. A control sample or a reference sample can also be a sample with a known amount of a detectable composition or a spiked sample.

The term “plant” is used in its broadest sense. It includes, but is not limited to, any species of woody, ornamental or decorative, crop or cereal, fruit or vegetable plant, and algae (e.g., Chlamydomonas reinhardtii). It also refers to a plurality of plant cells that is largely differentiated into a structure that is present at any stage of a plant's development. Such structures include, but are not limited to, a fruit, shoot, stem, leaf, flower petal, etc.

The term “plant tissue” includes differentiated and undifferentiated tissues of plants including those present in roots, shoots, leaves, inflorescences, anthers, pollen, ovaries, seeds and tumors, as well as cells in culture (e.g., single cells, protoplasts, embryos, callus, etc.). Plant tissue may be in planta, in organ culture, tissue culture, or cell culture.

The term “plant part” as used herein refers to a plant structure, a plant organ, or a plant tissue. In certain embodiments, the plant part may be a seed, pod, fruit, leaf, flower, stem, root, any part of the foregoing or a cell thereof, or a non-regenerable part or cell of a genetically modified or improved plant part. As used in this context, a “non-regenerable” part or cell of a genetically modified or improved plant or part thereof is a part or cell that itself cannot be induced to form a whole plant or cannot be induced to form a whole plant capable of sexual and/or asexual reproduction. In certain embodiments, the non-regenerable part or cell of the plant part is a part of a transgenic seed, pod, fruit, leaf, flower, stem or root or is a cell thereof.

Processed plant products that contain a detectable amount of a nucleotide segment, expressed RNA, and/or protein comprising a genetic modification disclosed herein are also provided. Such processed products include, but are not limited to, plant biomass, oil, meal, animal feed, flour, flakes, bran, lint, hulls, and processed seed. The processed product may be non-regenerable. The plant product can comprise commodity or other products of commerce derived from a transgenic plant or transgenic plant part, where the commodity or other products can be tracked through commerce by detecting a nucleotide segment, expressed RNA, and/or protein that comprises distinguishing portions of a genetic modification disclosed herein.

The term “plant cell” refers to a structural and physiological unit of a plant, comprising a protoplast and a cell wall. The plant cell may be in the form of an isolated single cell or a cultured cell, or as a part of a higher organized unit such as, for example, a plant tissue, a plant organ, or a whole plant. The term “plant cell culture” refers to cultures of plant units such as, for example, protoplasts, cells and cell clusters in a liquid medium or on a solid medium, cells in plant tissues and organs, microspores and pollen, pollen tubes, anthers, ovules, embryo sacs, zygotes and embryos at various stages of development.

The term “plant material” refers to leaves, stems, roots, inflorescences and flowers or flower parts, fruits, pollen, anthers, egg cells, zygotes, seeds, cuttings, cell or tissue cultures, or any other part or product of a plant.

A “plant organ” refers to a distinct and visibly structured and differentiated part of a plant, such as a root, stem, leaf, flower bud, inflorescence, spikelet, floret, seed or embryo.

The term “crop plant”, means in particular monocotyledons such as cereals (wheat, millet, sorghum, rye, triticale, oats, barley, teff, spelt, buckwheat, fonio and quinoa), rice, maize (corn), and/or sugar cane; or dicotyledon crops such as beet (such as sugar beet or fodder beet); fruits (such as pomes, stone fruits or soft fruits, for example apples, pears, plums, peaches, almonds, cherries, strawberries, raspberries or blackberries); leguminous plants (such as beans, lentils, peas or soybeans); oil plants (such as rape, mustard, poppy, olives, sunflowers, coconut, castor oil plants, cocoa beans or groundnuts); cucumber plants (such as marrows, cucumbers or melons); fiber plants (such as cotton, flax, hemp or jute); citrus fruit (such as oranges, lemons, grapefruit or mandarins); vegetables (such as spinach, lettuce, cabbages, carrots, tomatoes, potatoes, cucurbits or paprika); lauraceae (such as avocados, cinnamon or camphor); tobacco; nuts; coffee; tea; vines; hops; durian; bananas; natural rubber plants; and ornamentals (such as flowers, shrubs, broad-leaved trees or evergreens, for example conifers). This list does not represent any limitation.

The term “woody crop” or “woody plant” means a plant that produces wood as its structural tissue. Woody crops include trees, shrubs, or lianas. Examples of woody crops include, but are not limited to, thornless locust, hybrid chestnut, black walnut, Japanese maple, eucalyptus, casuarina, spruce, fir, pine, and flowering dogwood.

The term “improved growth” or “increased growth” is used herein in its broadest sense. It includes any improvement or enhancement in the process of plant growth and development. Examples of improved growth include, but are not limited to, increased photosynthetic efficiency, increased biomass, increased yield, increased seed number, increased seed weight, increased stem height, increased leaf area, increased root biomass, and increased plant dry weight,

The term “quantum yield” refers to the moles of CO₂ fixed per mole of quanta (photons) absorbed, or else the efficiency with which light is used to convert CO₂ into fixed carbon. The quantum yield of photosynthesis is derived from measurements of light intensity and rate of photosynthesis. As such, the quantum yield is a measure of the efficiency with which absorbed light produces a particular effect. The amount of photosynthesis performed in a plant cell or plant can be indirectly detected by measuring the amount of starch produced by the transgenic plant or plant cell. The amount of photosynthesis in a plant cell culture or a plant can also be detected using a CO₂ detector (e.g., a decrease or consumption of CO₂ indicates an increased level of photosynthesis) or an O₂ detector (e.g., an increase in the levels of O₂ indicates an increased level of photosynthesis (see, e.g., the methods described in Silva et al., Aquatic Biology 7:127-141, 2009; and Bai et al., Biotechnol. Lett.33:1675-1681, 2011). Photosynthesis can also be measured using radioactively labeled CO₂ (e.g., 14CO₂ and H₁₄CO₃⁻) (see, e.g., the methods described in Silva et al., Aquatic Biology 7:127-141, 2009, and the references cited therein). Photosynthesis can also be measured by detecting the chlorophyll fluorescence (e.g., Silva et al., Aquatic Biology 7:127-141, 2009, and the references cited therein). Additional methods for detecting photosynthesis in a plant are described in Zhang et al., Mol. Biol. Rep.38:4369-4379, 2011.

In the physical sciences, the term “relaxation” means the return of a perturbed system into equilibrium, usually from a high energy level to a low energy level. As used herein, the term “non-photochemical quenching relaxation” or “NPQ relaxation” refers to the process in which NPQ level decreases upon transition from high light intensity to low light intensity.

Reference to “about” a value or parameter herein refers to the usual error range for the respective value readily known to the skilled person in this technical field. Reference to “about” a value or parameter herein includes (and describes) aspects that are directed to that value or parameter per se. For example, description referring to “about X” includes description of “X.”

### Isolated Constructs or Expression Cassettes, Cells or Kits Including the Same, and Related Methods

A further aspect of the disclosure includes an expression vector or isolated DNA molecule including one or more gene editing components that target one or more uORF sequences of an endogenous PsbS gene, wherein the uORF sequence is edited by the one or more gene editing components to remove uORF translational repression. In another embodiment of this aspect, edited endogenous PsbS genes include SEQ ID NO: 2 (mutated TLS used in gmpsbs-atg1 construct), SEQ ID NO: 3 (mutated TLS used in gmpsbs-ttg6 construct), SEQ ID NO: 13 (mutated TLS used in vupsbs-atg4 construct), SEQ ID NO: 14 (mutated TLS used in vupsbs-ttg3 construct), and SEQ ID NO: 15 (mutated TLS used in vupsbs-ttg4 construct). In an additional embodiment of this aspect, the edited endogenous nucleotide sequences encoding the PsbS polypeptide include SEQ ID NO: 13 (mutated TLS used in vupsbs-atg4 construct) or SEQ ID NO: 15 (mutated TLS used in vupsbs-ttg4 construct).

An additional aspect of the disclosure includes an expression vector or isolated DNA molecule including one or more gene editing components that target one or more uORF sequences of an endogenous VDE gene, wherein the uORF sequence is edited by the one or more gene editing components to remove uORF translational repression. In yet another embodiment of this aspect, edited endogenous VDE genes include SEQ ID NO: 5 (mutated TLS used in gmvde-atg1 construct), SEQ ID NO: 6 (mutated TLS used in gmvde-atg2 construct), and SEQ ID NO: 7 (mutated TLS used in gmvde-atg3 construct). In an additional embodiment of this aspect, the edited endogenous nucleotide sequences encoding the VDE polypeptide include SEQ ID NO: 5 (mutated TLS used in gmvde-atg1 construct).

Yet another aspect of the disclosure includes an expression vector or isolated DNA molecule including one or more gene editing component that target one or more uORF sequences of an endogenous ZEP gene, wherein the uORF sequence is edited by the one or more gene editing components to remove uORF translational repression. In a further embodiment of this aspect, edited endogenous ZEP genes include SEQ ID NO: 9 (mutated TLS used in gmzep-ctg1 construct), SEQ ID NO: 10 (mutated TLS used in gmzep-ctg2 construct), SEQ ID NO: 11 (mutated TLS used in gmzep-ttg1 construct), SEQ ID NO: 17 (mutated TLS used in vuzep-atg1 construct), SEQ ID NO: 18 (mutated TLS used in vuzep-atg2 construct), and SEQ ID NO: 19 (mutated TLS used in vuzep-atg3 construct). In an additional embodiment of this aspect, the edited endogenous nucleotide sequences encoding the ZEP polypeptide include SEQ ID NO: 11 (mutated TLS used in gmzep-ttg1 construct).

In a further embodiment of any of the preceding aspects that have an expression vector or isolated DNA molecule, the one or more gene editing components are selected from the group of a ribonucleoprotein complex that targets the one or more uORF sequences; a vector including a TALEN protein encoding sequence, wherein the TALEN protein targets the one or more uORF sequences; a vector including a ZFN protein encoding sequence, wherein the ZFN protein targets the one or more uORF sequences; an oligonucleotide donor (ODN), wherein the ODN targets the one or more uORF sequences; or a vector including a CRISPR/Cas enzyme encoding sequence and a targeting sequence, wherein the targeting sequence targets the one or more uORF sequences.

Some aspects of the present disclosure relate to a bacterial cell or an Agrobacterium cell including the expression vector or isolated DNA molecule of any of the preceding embodiments.

Further aspects of the present disclosure relate to a composition or kit including the expression vector or isolated DNA molecule of any of the preceding embodiments, or the bacterial cell or the Agrobacterium cell of the preceding embodiment.

Additional aspects of the present disclosure relate to genetically modified plant, plant part, plant cell, or seed including the expression vector or isolated DNA molecule of any of the preceding embodiments. The plant part or plant cell may be non-regenerable.

Further aspects of the present disclosure relate to a composition or kit including the genetically modified plant of any of the preceding embodiments, the genetically modified plant, plant part, plant cell, or seed of the preceding embodiment, or the genetically modified plant produced by the method of any of the preceding embodiments.

Still further aspects of the present disclosure relate to methods of increasing the rate of relaxation of NPQ in a plant, including: introducing a genetic alteration via the expression vector or isolated DNA molecule of any one of the preceding embodiments, or a combination thereof, to a cell. In a further embodiment of this aspect, the cell is a plant cell.

### Transient Screening Methods

Yet another aspect of the disclosure includes transient screening methods to study gene expression, including: a) providing one or more dual-fluorescence expression vectors including a test insert to produce one or more test vectors and at least one dual-fluorescence expression vector including a control insert to produce at least one control vector, wherein the dual-fluorescence expression vectors include a first fluorescent reporter and a second fluorescent reporter; b) introducing the one or more test vectors into a first leaf tissue and introducing the at least one control vector into a second leaf tissue, wherein the first leaf tissue and the second leaf tissue are of the same plant variety; d) excising leaf portions from the first leaf tissue and from the second leaf tissue 3 days post injection; e) placing the leaf portions abaxially in a multiwell plate; and f) measuring fluorescence on a monochromator-based plate reader in two separate channels, wherein a first channel is used to obtain a signal from the first fluorescent reporter and a second channel is used to obtain a signal from the second fluorescent reporter. In a further embodiment of this aspect, the plant variety is an N. benthamiana variety. In an additional embodiment of this aspect, which may be combined with any of the preceding embodiments, step b) includes: b-1) transforming a first Agrobacterium tumefaciens strain with the one or more test vectors and transforming a second A. tumefaciens strain with the at least one control vector; and b-2) injecting the first A. tumefaciens strain harboring the one or more test vectors into the first leaf tissue and injecting the second A. tumefaciens strain into the second leaf tissue. In a further embodiment of this aspect, which may be combined with any of the preceding embodiments, the first and/or second A. tumefaciens strain is C58C1. In yet another embodiment of this aspect, which may be combined with any of the preceding embodiments, the leaf portions are discs. In still another embodiment of this aspect, which may be combined with any of the preceding embodiments, the multiwall plate is a 96-well plate or a 384-well plate. In an additional embodiment of this aspect, which may be combined with any of the preceding embodiments, the first fluorescent reporter is a green fluorescent reporting and the second fluorescent reporter is a red fluorescent reporter. In yet another embodiment of this aspect, the first fluorescent reporter is mNeonGreen and the second fluorescent reporter is tdTomato. Still another embodiment of this aspect, which may be combined with any of the preceding embodiments, further includes step (g) normalizing the signal obtained from the first fluorescent reporter in step (f) to the signal obtained from the second fluorescent reporter in step (f) to reduce noise, and to produce a graph of normalized results comparing results obtained from the one or more test vectors with results obtained from the at least one control vector to determine expression differences between the one or more test vectors and the at least one control vector. In a further embodiment of this aspect, comparing results further includes determining p-values using a nonparametric bootstrap for two-sample t-testing that is adjusted for multiple testing. In yet another embodiment of the aspect, which may be combined with any of the preceding embodiments, the test insert includes a mutated TLS, optionally wherein the mutated TLS is mutated in one or more uORFs, and wherein the control insert includes a wild-type TLS. Exemplary wild-type and mutated TLSs are provided in the section below.

### Molecular Biological Methods to Produce Transgenic Plants, Plant Parts, and Plant Cells

One aspect of the present disclosure provides transgenic plants, plant parts, or plant cells including one or more edited open reading frames (uORFs) in endogenous nucleotide sequences encoding photosystem II subunit S (PsbS), zeaxanthin epoxidase (ZEP), or violaxanthin de-epoxidase (VDE).

Exemplary PsbS homologs include Glycine max PsbS1 (GmPsbS1) and Vigna unguiculata PsbS (VuPsbS). The wild-type transcript leader sequence (TLS) of GmPsbS1 is provided in SEQ ID NO: 1 (with overhangs for cloning) and SEQ ID NO: 22 (without overhangs for cloning). TLSs of GmPsbS1 mutated at two different uORFs, ATG1 and TTG6, are provided in SEQ ID NO: 2 (mutated TLS used in gmpsbs-atg1 construct) and SEQ ID NO: 3 (mutated TLS used in gmpsbs-ttg6 construct). In both of these mutated TLSs, the start codon of the uORF was replaced with an AAA triplet. The wild-type transcript leader sequence (TLS) of VuPsbS is provided in SEQ ID NO: 12 (with overhangs for cloning) and SEQ ID NO: 25 (without overhangs for cloning). TLSs of VuPsbS mutated at three different uORFs, ATG4, TTG3, and TTG4, are provided in SEQ ID NO: 13 (mutated TLS used in vupsbs-atg4 construct), SEQ ID NO: 14 (mutated TLS used in vupsbs-ttg3 construct), and SEQ ID NO: 15 (mutated TLS used in vupsbs-ttg4 construct). In SEQ ID NO: 13 (mutated TLS used in vupsbs-atg4 construct), the start codon of the uORF was replaced with an AAA triplet. In SEQ ID NO: 14 (mutated TLS used in vupsbs-ttg3 construct), and SEQ ID NO: 15 (mutated TLS used in vupsbs-ttg4 construct), the start codons of each of the uORFs were replaced with a TTT triplet. PsbS homolog sequences from multiple plant species include the nucleotide sequence SEQ ID NO: 27, and the polypeptide sequences SEQ ID NO: 30, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, and SEQ ID NO: 51.

Exemplary ZEP homologs include Glycine max ZEP3 (GmZEP3) and Vigna unguiculata ZEP1 (VuZEP1). The wild-type transcript leader sequence (TLS) of GmZEP3 is provided in SEQ ID NO: 8 (with overhangs for cloning) and SEQ ID NO: 24 (without overhangs for cloning). TLSs of GmZEP3 mutated at three different uORFs, CTG1, CTG2, and TTG1, are provided in SEQ ID NO: 9 (mutated TLS used in gmzep-ctg1 construct), SEQ ID NO: 10 (mutated TLS used in gmzep-ctg2 construct), and SEQ ID NO: 11 (mutated TLS used in gmzep-ttg1 construct). In SEQ ID NO: 9 (mutated TLS used in gmzep-ctg1 construct) and SEQ ID NO: 10 (mutated TLS used in gmzep-ctg2 construct), the start codon of the uORF was replaced with an CCC triplet. In SEQ ID NO: 11 (mutated TLS used in gmzep-ttg1 construct), the start codon of the uORF was replaced with a TTT triplet. The wild-type transcript leader sequence (TLS) of VuZEP1 is provided in SEQ ID NO: 16 (with overhangs for cloning) and SEQ ID NO: 26 (without overhangs for cloning). TLSs of VuZEP1 mutated at three different uORFs, ATG1, ATG2, and ATG3, are provided in SEQ ID NO: 17 (mutated TLS used in vuzep-atg1 construct), SEQ ID NO: 18 (mutated TLS used in vuzep-atg2 construct), and SEQ ID NO: 19 (mutated TLS used in vuzep-atg3 construct). In all three of these mutated TLSs, the start codon of the uORF was replaced with an AAA triplet. ZEP homolog sequences from multiple plant species include the nucleotide sequence SEQ ID NO: 29, and the polypeptide sequences SEQ ID NO: 32, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, and SEQ ID NO: 88.

Glycine max VDE1 (GmVDE1) is an exemplary VDE homolog. The wild-type transcript leader sequence (TLS) of GmVDE1 is provided in SEQ ID NO: 4 (with overhangs for cloning) and SEQ ID NO: 23 (without overhangs for cloning). TLSs of GmVDE1 mutated at three different uORFs, ATG1, ATG2, and ATG3, are provided in SEQ ID NO: 5 (mutated TLS used in gmvde-atg1 construct), SEQ ID NO: 6 (mutated TLS used in gmvde-atg2 construct), and SEQ ID NO: 7 (mutated TLS used in gmvde-atg3 construct). In each of these mutated TLSs, the start codon of the uORF was replaced with an AAA triplet. VDE homolog sequences from multiple plant species include the nucleotide sequence SEQ ID NO: 28, and the polypeptide sequences SEQ ID NO: 31, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, and SEQ ID NO: 70.

Transformation and generation of genetically altered monocotyledonous and dicotyledonous plant cells is well known in the art. See, e.g., Weising, et al., Ann. Rev. Genet. 22:421-477 (1988); U.S. Pat. No. 5,679,558; Agrobacterium Protocols, ed: Gartland, Humana Press Inc. (1995); Wang, et al. Acta Hort. 461:401-408 (1998), and Broothaerts, et al. Nature 433:629-633 (2005). The choice of method varies with the type of plant to be transformed, the particular application and/or the desired result. The appropriate transformation technique is readily chosen by the skilled practitioner.

Any methodology known in the art to delete, insert or otherwise modify the cellular DNA (e.g., genomic DNA and organelle DNA) can be used in practicing the compositions, methods, and processes disclosed herein. As an example, the CRISPR/Cas-9 system and related systems (e.g., TALEN, ZFN, ODN, etc.) may be used to insert a heterologous gene to a targeted site in the genomic DNA or substantially edit an endogenous gene to express the heterologous gene or to modify the promoter to increase or otherwise alter expression of an endogenous gene through, for example, removal of repressor binding sites or introduction of enhancer binding sites. For example, a disarmed Ti plasmid, containing a genetic construct for deletion or insertion of a target gene, in Agrobacterium tumefaciens can be used to transform a plant cell, and thereafter, a transformed plant can be regenerated from the transformed plant cell using procedures described in the art, for example, in EP 0116718, EP 0270822, PCT publication WO 84/02913 and published European Patent application (“EP”) 0242246. Ti-plasmid vectors each contain the gene between the border sequences, or at least located to the left of the right border sequence, of the T-DNA of the Ti-plasmid. Of course, other types of vectors can be used to transform the plant cell, using procedures such as direct gene transfer (as described, for example in EP 0233247), pollen mediated transformation (as described, for example in EP 0270356, PCT publication WO 85/01856, and U.S. Pat. No. 4,684,611), plant RNA virus-mediated transformation (as described, for example in EP 0 067 553 and U.S. Pat. No. 4,407,956), liposome-mediated transformation (as described, for example in U.S. Pat. No. 4,536,475), and other methods such as the methods for transforming certain lines of corn (e.g., U.S. Pat. No. 6,140,553; Fromm et al., Bio/Technology (1990) 8, 833-839); Gordon-Kamm et al., The Plant Cell, (1990) 2, 603-618), rice (Shimamoto et al., Nature, (1989) 338, 274-276; Datta et al., Bio/Technology, (1990) 8, 736-740), and the method for transforming monocots generally (PCT publication WO 92/09696). For cotton transformation, the method described in PCT patent publication WO 00/71733 can be used. For soybean transformation, reference is made to methods known in the art, e.g., Hinchee et al. (Bio/Technology, (1988) 6, 915) and Christou et al. (Trends Biotech, (1990) 8, 145) or the method of WO 00/42207.

Genetically altered plants of the present disclosure can be used in a conventional plant breeding scheme to produce more genetically altered plants with the same characteristics, or to introduce the genetic alteration(s) in other varieties of the same or related plant species. Seeds, which are obtained from the altered plants, preferably contain the genetic alteration(s) as a stable insert in chromosomal DNA or as modifications to an endogenous gene or promoter. Plants including the genetic alteration(s) in accordance with this disclosure include plants including, or derived from, root stocks of plants including the genetic alteration(s) of this disclosure, e.g., fruit trees or ornamental plants. Hence, any non-transgenic grafted plant parts inserted on a transformed plant or plant part are included in this disclosure.

Genetic alterations of the disclosure, including in an expression vector or expression cassette, which result in the expression of an introduced gene or altered expression of an endogenous gene will typically utilize a plant-expressible promoter. A ‘plant-expressible promoter’ as used herein refers to a promoter that ensures expression of the genetic alteration(s) of this disclosure in a plant cell. Examples of constitutive promoters that are often used in plant cells are the cauliflower mosaic (CaMV) 35S promoter (KAY et al. Science, 236, 4805, 1987), the minimal CaMV 35S promoter (Benfey & Chua, Science, (1990) 250, 959-966), various other derivatives of the CaMV 35S promoter, the figwort mosaic virus (FMV) promoter (Richins, et al., Nucleic Acids Res. (1987) 15:8451-8466), the maize ubiquitin promoter (CHRISTENSEN & QUAIL, Transgenic Res, 5, 213-8, 1996), the polyubiquitin promoter (Ljubql, MAEKAWA et al. Mol Plant Microbe Interact. 21, 375-82, 2008), the vein mosaic cassava virus promoter (International Application WO 97/48819), and the Arabidopsis UBQ10 promoter, Norris et al. Plant Mol. Biol. 21, 895-906, 1993).

Additional examples of promoters directing constitutive expression in plants are known in the art and include: the strong constitutive 35S promoters (the “35S promoters”) of the cauliflower mosaic virus (CaMV), e.g., of isolates CM 1841 (Gardner et al., Nucleic Acids Res, (1981) 9, 2871-2887), CabbB S (Franck et al., Cell (1980) 21, 285-294) and CabbB JI (Hull and Howell, Virology, (1987) 86, 482-493); promoters from the ubiquitin family (e.g., the maize ubiquitin promoter of Christensen et al., Plant Mol Biol, (1992) 18, 675-689), the gos2 promoter (de Pater et al., The Plant J (1992) 2, 834-844), the emu promoter (Last et al., Theor Appl Genet, (1990) 81, 581-588), actin promoters such as the promoter described by An et al. (The Plant J, (1996) 10, 107), the rice actin promoter described by Zhang et al. (The Plant Cell, (1991) 3, 1155-1165); promoters of the figwort mosaic virus (FMV) (Richins, et al., Nucleic Acids Res. (1987) 15:8451-8466), promoters of the Cassava vein mosaic virus (WO 97/48819; Verdaguer et al., Plant Mol Biol, (1998) 37, 1055-1067), the pPLEX series of promoters from Subterranean Clover Stunt Virus (WO 96/06932, particularly the S4 or S7 promoter), an alcohol dehydrogenase promoter, e.g., pAdh1S (GenBank accession numbers X04049, X00581), and the TR1′ promoter and the TR2′ promoter (the “TR1′ promoter” and “TR2′ promoter”, respectively) which drive the expression of the 1′ and 2′ genes, respectively, of the T DNA (Velten et al., EMBO J, (1984) 3, 2723-2730).

Alternatively, a plant-expressible promoter can be a tissue-specific promoter, i.e., a promoter directing a higher level of expression in some cells or tissues of the plant, e.g., in green tissues (such as the promoter of the chlorophyll a/b binding protein (Cab)). The plant Cab promoter (Mitra et al., Planta, (2009) 5: 1015-1022) has been described to be a strong bidirectional promoter for expression in green tissue (e.g., leaves and stems) and is useful in one embodiment of the current disclosure. These plant-expressible promoters can be combined with enhancer elements, they can be combined with minimal promoter elements, or can include repeated elements to ensure the expression profile desired.

Additional non-limiting examples of tissue-specific promoters include the maize metallothionein promoter (DE FRAMOND et al, FEBS 290, 103-106, 1991; Application EP 452269), the chitinase promoter (SAMAC et al. Plant Physiol 93, 907-914, 1990), the maize ZRP2 promoter (U.S. Pat. No. 5,633,363), the tomato LeExtl promoter (Bucher et al. Plant Physiol. 128, 911-923, 2002), the glutamine synthetase soybean root promoter (HIREL et al. Plant Mol. Biol. 20, 207-218, 1992), the RCC3 promoter (PCT Application WO 2009/016104), the rice antiquitin promoter (PCT Application WO 2007/076115), the LRR receptor kinase promoter (PCT application WO 02/46439), and the Arabidopsis pCO2 promoter (HEIDSTRA et al, Genes Dev. 18, 1964-1969, 2004). Further non-limiting examples of tissue-specific promoters include the RbcS2B promoter, RbcS1B promoter, RbcS3B promoter, LHB1B1 promoter, LHB1B2 promoter, cabl promoter, and other promoters described in Engler et al., ACS Synthetic Biology, DOI: 10.1021/sb4001504, 2014. These plant promoters can be combined with enhancer elements, they can be combined with minimal promoter elements, or can include repeated elements to ensure the expression profile desired.

In some embodiments, further genetic alterations to increase expression in plant cells can be utilized. For example, an intron at the 5′ end or 3′ end of an introduced gene, or in the coding sequence of the introduced gene, e.g., the hsp70 intron. Other such genetic elements can include, but are not limited to, promoter enhancer elements, duplicated or triplicated promoter regions, 5′ leader sequences different from another transgene or different from an endogenous (plant host) gene leader sequence, 3′ trailer sequences different from another transgene used in the same plant or different from an endogenous (plant host) trailer sequence.

An introduced gene of the present disclosure can be inserted in host cell DNA so that the inserted gene part is upstream (i.e., 5′) of suitable 3′ end transcription regulation signals (i.e., transcript formation and polyadenylation signals). This is preferably accomplished by inserting the gene in the plant cell genome (nuclear or chloroplast). Preferred polyadenylation and transcript formation signals include those of the nopaline synthase gene (Depicker et al., J. Molec Appl Gen, (1982) 1, 561-573), the octopine synthase gene (Gielen et al., EMBO J, (1984) 3:835-845), the SCSV or the Malic enzyme terminators (Schunmann et al., Plant Funct Biol, (2003) 30:453-460), and the T DNA gene 7 (Velten and Schell, Nucleic Acids Res, (1985) 13, 6981-6998), which act as 3′ untranslated DNA sequences in transformed plant cells. In some embodiments, one or more of the introduced genes are stably integrated into the nuclear genome. Stable integration is present when the nucleic acid sequence remains integrated into the nuclear genome and continues to be expressed (i.e., detectable mRNA transcript or protein is produced) throughout subsequent plant generations. Stable integration into the nuclear genome can be accomplished by any known method in the art (e.g., microparticle bombardment, Agrobacterium-mediated transformation, CRISPR/Cas9, electroporation of protoplasts, microinjection, etc.). Targeted editing approaches, e.g., prime editing or base editing may also be used.

The term recombinant or modified nucleic acids refers to polynucleotides which are made by the combination of two otherwise separated segments of sequence accomplished by the artificial manipulation of isolated segments of polynucleotides by genetic engineering techniques or by chemical synthesis. In so doing one may join together polynucleotide segments of desired functions to generate a desired combination of functions.

As used herein, the term “overexpression” refers to increased expression (e.g., of mRNA, polypeptides, etc.) relative to expression in a wild-type organism (e.g., plant) as a result of genetic modification and can refer to expression of heterologous genes at a sufficient level to achieve the desired result such as increased yield. In some embodiments, the increase in expression is a slight increase of about 10% more than expression in wild-type. In some embodiments, the increase in expression is an increase of 50% or more (e.g., 60%, 70%, 80%, 100%, etc.) relative to expression in wild-type. In some embodiments, an endogenous gene is upregulated. In some embodiments, an exogenous gene is upregulated by virtue of being expressed. Upregulation of a gene in plants can be achieved through any known method in the art, including but not limited to, the use of constitutive promoters with inducible response elements added, inducible promoters, high expression promoters (e.g., PsaD promoter) with inducible response elements added, enhancers, transcriptional and/or translational regulatory sequences, codon optimization, modified transcription factors, and/or mutant or modified genes that control expression of the gene to be upregulated in response to a stimulus such as cytokinin signaling.

Where a recombinant nucleic acid is intended for expression, cloning, or replication of a particular sequence, DNA constructs prepared for introduction into a host cell will typically include a replication system (e.g., vector) recognized by the host, including the intended DNA fragment encoding a desired polypeptide, and can also include transcription and translational initiation regulatory sequences operably linked to the polypeptide-encoding segment. Additionally, such constructs can include cellular localization signals (e.g., plasma membrane localization signals). In preferred embodiments, such DNA constructs are introduced into a host cell's genomic DNA, chloroplast DNA or mitochondrial DNA.

In some embodiments, a non-integrated expression system can be used to induce expression of one or more introduced genes. Expression systems (expression vectors) can include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Signal peptides can also be included where appropriate from secreted polypeptides of the same or related species, which allow the protein to cross and/or lodge in cell membranes, cell wall, or be secreted from the cell.

Selectable markers useful in practicing the methodologies disclosed herein can be positive selectable markers. Typically, positive selection refers to the case in which a genetically altered cell can survive in the presence of a toxic substance only if the recombinant polynucleotide of interest is present within the cell. Negative selectable markers and screenable markers are also well known in the art and are contemplated by the present disclosure. One of skill in the art will recognize that any relevant markers available can be utilized in practicing the compositions, methods, and processes disclosed herein.

Screening and molecular analysis of recombinant strains of the present disclosure can be performed utilizing nucleic acid hybridization techniques. Hybridization procedures are useful for identifying polynucleotides, such as those modified using the techniques described herein, with sufficient homology to the subject regulatory sequences to be useful as taught herein. The particular hybridization techniques are not essential to this disclosure. As improvements are made in hybridization techniques, they can be readily applied by one of skill in the art. Hybridization probes can be labeled with any appropriate label known to those of skill in the art. Hybridization conditions and washing conditions, for example temperature and salt concentration, can be altered to change the stringency of the detection threshold. See, e.g., Sambrook et al. (1989) vide infra or Ausubel et al. (1995) Current Protocols in Molecular Biology, John Wiley & Sons, NY, N.Y., for further guidance on hybridization conditions.

Additionally, screening and molecular analysis of genetically altered strains, as well as creation of desired isolated nucleic acids can be performed using Polymerase Chain Reaction (PCR). PCR is a repetitive, enzymatic, primed synthesis of a nucleic acid sequence. This procedure is well known and commonly used by those skilled in this art (see Mullis, U.S. Pat. Nos. 4,683,195, 4,683,202, and 4,800,159; Saiki et al. (1985) Science 230:1350-1354). PCR is based on the enzymatic amplification of a DNA fragment of interest that is flanked by two oligonucleotide primers that hybridize to opposite strands of the target sequence. The primers are oriented with the 3′ ends pointing towards each other. Repeated cycles of heat denaturation of the template, annealing of the primers to their complementary sequences, and extension of the annealed primers with a DNA polymerase result in the amplification of the segment defined by the 5′ ends of the PCR primers. Because the extension product of each primer can serve as a template for the other primer, each cycle essentially doubles the amount of DNA template produced in the previous cycle. This results in the exponential accumulation of the specific target fragment, up to several million-fold in a few hours. By using a thermostable DNA polymerase such as the Taq polymerase, which is isolated from the thermophilic bacterium Thermus aquaticus, the amplification process can be completely automated. Other enzymes which can be used are known to those skilled in the art.

Nucleic acids and proteins of the present disclosure can also encompass homologs of the specifically disclosed sequences. Homology (e.g., sequence identity) can be 50%-100%. In some instances, such homology is greater than 80%, greater than 85%, greater than 90%, or greater than 95%. The degree of homology or identity needed for any intended use of the sequence(s) is readily identified by one of skill in the art. As used herein percent sequence identity of two nucleic acids is determined using an algorithm known in the art, such as that disclosed by Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the BLASTN, BLASTP, and BLASTX, programs of Altschul et al. (1990) J. Mol. Biol. 215:402-410. BLAST nucleotide searches are performed with the BLASTN program, score=100, wordlength=12, to obtain nucleotide sequences with the desired percent sequence identity. To obtain gapped alignments for comparison purposes, Gapped BLAST is used as described in Altschul et al. (1997) Nucl. Acids. Res. 25:3389-3402. When utilizing BLAST and Gapped BLAST programs, the default parameters of the respective programs (BLASTN and BLASTX) are used. See www.ncbi.nih.gov. One of skill in the art can readily determine in a sequence of interest where a position corresponding to amino acid or nucleic acid in a reference sequence occurs by aligning the sequence of interest with the reference sequence using the suitable BLAST program with the default settings (e.g., for BLASTP: Gap opening penalty: 11, Gap extension penalty: 1, Expectation value: 10, Word size: 3, Max scores: 25, Max alignments: 15, and Matrix: blosum62; and for BLASTN: Gap opening penalty: 5, Gap extension penalty: 2, Nucleic match: 1, Nucleic mismatch—3, Expectation value: 10, Word size: 11, Max scores: 25, and Max alignments: 15).

Preferred host cells are plant cells. Recombinant host cells, in the present context, are those which have been genetically modified to contain an isolated nucleic molecule, contain one or more deleted or otherwise non-functional genes normally present and functional in the host cell, or contain one or more genes to produce at least one recombinant protein. The nucleic acid(s) encoding the protein(s) of the present disclosure can be introduced by any means known to the art, which is appropriate for the particular type of cell, including without limitation, transformation, lipofection, electroporation or any other methodology known by those skilled in the art.

“Isolated”, “isolated DNA molecule” or an equivalent term or phrase is intended to mean that the DNA molecule or other moiety is one that is present alone or in combination with other compositions but altered from or not within its natural environment. For example, nucleic acid elements such as a coding sequence, intron sequence, untranslated leader sequence, promoter sequence, transcriptional termination sequence, and the like, that are naturally found within the DNA of the genome of an organism are not considered to be “isolated” so long as the element is within the genome of the organism and at the location within the genome in which it is naturally found. However, each of these elements, and subparts of these elements, would be “isolated” from its natural setting within the scope of this disclosure so long as the element is not within the genome of the organism in which it is naturally found, the element is altered from its natural form, or the element is not at the location within the genome in which it is naturally found. Similarly, a nucleotide sequence encoding a protein or any naturally occurring variant of that protein would be an isolated nucleotide sequence so long as the nucleotide sequence was not within the DNA of the organism from which the sequence encoding the protein is naturally found in its natural location or if that nucleotide sequence was altered from its natural form. A synthetic nucleotide sequence encoding the amino acid sequence of the naturally occurring protein would be considered to be isolated for the purposes of this disclosure. For the purposes of this disclosure, any transgenic nucleotide sequence, i.e., the nucleotide sequence of the DNA inserted into the genome of the cells of a plant, alga, fungus, or bacterium, or present in an extrachromosomal vector, would be considered to be an isolated nucleotide sequence whether it is present within the plasmid or similar structure used to transform the cells, within the genome of the plant or bacterium, or present in detectable amounts in tissues, progeny, biological samples or commodity products derived from the plant or bacterium.

Unless otherwise indicated, elements positively recited herein can also be understood to include a negative recitation of said elements. As such, elements that are positively recited in the specification can provide the basis for negative recitations of such elements and may be explicitly excluded in the claims as, for example, a negative limitation or an exclusionary proviso.

Having generally described the compositions, methods, and processes of this disclosure, the same will be better understood by reference to certain specific examples, which are included herein to further illustrate the disclosure and are not intended to limit the scope of the invention as defined by the claims.

### Examples

The present disclosure is described in further detail in the following examples which are not in any way intended to limit the scope of the disclosure as claimed. The attached figures are meant to be considered as integral parts of the specification and description of the disclosure. The following example is offered to illustrate, but not to limit the claimed disclosure.

### Example 1: Validation of Workflow for Editing uORFs and Validating Effect of Editing in A. thaliana BRI1 and L. sativa GGP2

The following example describes a workflow for editing uORFs in multiple plant species by using a single-step assembly of a vector system and transient expression in leaf tissue. This allowed the effect of the editing to be validated without the use of protoplasts.

### Materials and Methods

### Construct Design

In order to validate the workflow, gene targets were selected that are known to show strong overexpression when uORFs are disrupted: Arabidopsis thaliana BRASSINOSTEROID-INSENSITIVE 1 (AtBRI1) and Lactuca sativa GDP-l-galactose phosphorylase 2 (LsGGP2) (Si et al., (2020), Manipulating Gene Translation in Plants by CRISPR-Cas9-Mediated Genome Editing of Upstream Open Reading Frames, Nature Protocols 15: 338-363). In A. thaliana, the overexpression of AtBRI1 reduces inhibition of A. thaliana hypocotyl growth by brassinazole, a chemical that inhibits brassinosteroid (BR) biosynthesis. Si et al. found that the mutated uORF variant of AtBRI1 had a 200% increase in signal, which was statistically significant compared to the control. In addition, the edited plants showed longer hypocotyls in the presence of brassinazole. In L. sativa, LsGGP2 encodes a key enzyme in vitamin C (ascorbic acid) biosynthesis. Si et al. found that the mutated uORF variant of LsGGP2 had a 600% increase in signal, which was statistically significant compared to the control. The edited plants also had 100-300% higher ascorbic acid content.

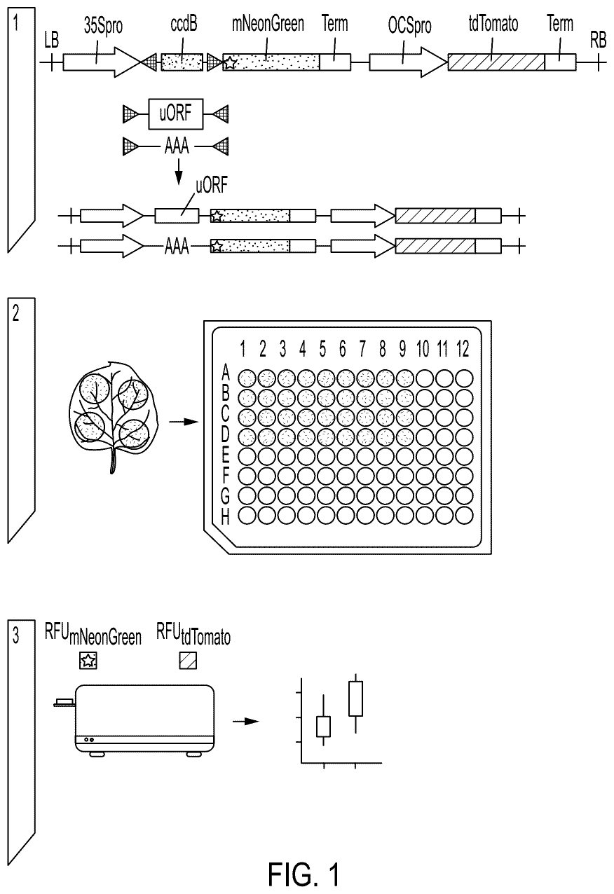

For each of AtBRI1 and LsGGP2, constructs were prepared that incorporated either a wild-type transcript leader sequence (TLS), i.e., a TLS with a functional, wild-type uORF, or a TLS mutated to lack a functional uORF. Mutation was done by replacing the start codon of the uORF with an AAA nucleotide triplet. SEQ ID NO: 20 provides the wild-type TLS of AtBRI1, and SEQ ID NO: 21 provides the wild-type TLS of LsGGP2. Cloning was optimized to allow single-step assembly of expression vectors containing these constructs. Diagrams of the constructs used are illustrated in step 1 of FIG. 1. Two reporter genes, mNeonGreen and tdTomato, were present in each construct.

### Transient Reporter Assay

A dual reporter assay was used to quantify transient expression, as quantifying relative signals between two reporters accounted for differences in transformation and expression efficiency between treatments. The assay was developed on the basis of previous assay methods published by Pasin et al. and Promega (Pasin, Fabio, Satish Kulasekaran, Paolo Natale, Carmen Simon Mateo, and Juan Antonio Garcia. 2014. “Rapid Fluorescent Reporter Quantification by Leaf Disc Analysis and Its Application in Plant-Virus Studies.” Plant Methods 10 (1): 22. doi[dot]org/10[dot]1186/1746-4811-10-22; and Dual-Luciferase® Reporter Assay—Promega: www[dot]promega[dot]com/products/luciferase-assays/reporter-assays/dual_luciferase-reporter-assay-system/). The two reporters mNeonGreen (green fluorescent protein variant) and tdTomato (red fluorescent protein variant) were chosen for use together, as their excitation and emission maxima were distinguishable from each other as well as from chlorophyll. The fluorescent protein coding sequences were codon-optimized for N. benthamiana. The utilization of a monochromator-based plate reader for the quantification of fluorescence in leaf discs allowed parallel analysis of multiple samples with little preparatory requirements. Excitation and emission wavelengths as well as cutoff for the measurement of mNeonGreen and tdTomato expressed in Nicotiana benthamiana leaf tissue were adjusted to the settings in Table 1, below (λ=wavelength).

TABLE 1
Wavelength criteria for plate reader quantification

| Fluorescent | Excitation λ | Emission λ | Emission cutoff |
|---|---|---|---|
| protein | (nm) | (nm) | (nm) |
| mNeonGreen | 490 | 520 | 515 |
| tdTomato | 540 | 595 | 590 |

Dual-fluorescence expression vectors were transformed into Agrobacterium tumefaciens C58C1. Then, A. tumefaciens C58C1 harboring a construct was injected into 4-6-week-old Nicotiana benthamiana leaf tissue (instead of protoplasts, as used in Si et al.). Three days post infection, 6 mm discs of the leaf tissue were sampled and placed abaxially into microplate wells in a 96-well plate. The microplate wells were then assayed for fluorescence by a monochromator-based microplate reader in two separate, as illustrated in FIG. 1.

The signal obtained from mNeonGreen was then normalized to the tdTomato signal to reduce noise stemming from pipetting error, differences in transformation efficiency and cell viability. Relative fluorescence ratios (unitless) were obtained by dividing mNeonGreen by tdTomato emission signals, both measured in relative fluorescence units (RFU, in units nm/nm). Obtained relative fluorescence values were normalized to the mean values of the constructs with wild-type transcript leaders. The sample size for each construct was n=13-14, indicating 13-14 independently, transiently transformed N. benthamiana plants (1 leaf disc per transformation). The difference in mean between the performance of the construct with a functional uORF and the construct without a functional uORF was analyzed with a nonparametric bootstrap for two-sample t-testing, with p-values corrected for multiple testing after Benjamini & Hochberg (1995, Journal of the royal statistical society series b-methodological, 57: 289-300), using the modified Bonferroni correction to control the false discovery rate.

### Results

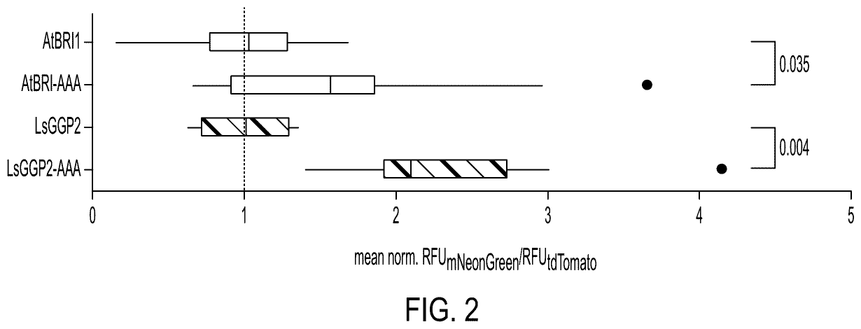

FIG. 2 shows the results of tests comparing AtBRI1 and LsGGP2 with disrupted uORFs to AtBRI1 and LsGGP2 without disrupted uORFs. Both genes were successfully and significantly overexpressed through uORF disruption. As shown in FIG. 2, there was a 67% increase in AtBRI1 expression in the construct with uORF disruption (AtBRI1-AAA) as compared to the construct without uORF disruption (AtBRI1). Similarly, there was a 157% increase in LsGGP2 expression for the construct with uORF disruption (LsGGP1-AAA) as compared to the construct without uORF disruption (LsGGP2). The successful overexpression of both AtBRI1 and LsGGP2 validated this workflow and demonstrated its utility in assessing uORF function.

### Example 2: Targeted Editing of uORFs of PsbS Across Multiple Plant Species

The following example describes the editing of uORFs of PsbS genes across multiple plant species. PsbS is a gene involved in non-photochemical quenching (NPQ), where excess light energy is dissipated as heat, and editing PsbS uORFs to overexpress PsbS had the potential to accelerate plant recovery from NPQ. Improving the rate of NPQ recovery has been shown to increase biomass yield over the course of a field season in important crop plants.

### Materials and Methods

Identifying PsbS Targets for uORF Constructs

To identify PsbS gene targets for uORF constructs, the tool Phytozome (phytozome-next[dot]jgi[dot]doe[dot]gov) was used. Photosystem II subunit S (PsbS) was selected for testing, due to its importance for non-photochemical quenching and intrinsic water use efficiency. PsbS is a pH sensor protein that plays a crucial role in plant photoprotection by detecting thylakoid lumen acidification in excess light conditions via two lumen-faced glutamates. PsbS genes in Glycine max (soybean) and Vigna unguiculata (cowpea) were selected for testing. Specifically, the soybean gene GmPsbS1 (Glyma.04G249700) and the cowpea gene VuPsbS (Vigun09g165900) were selected.

### Producing Glycine max PsbS Constructs

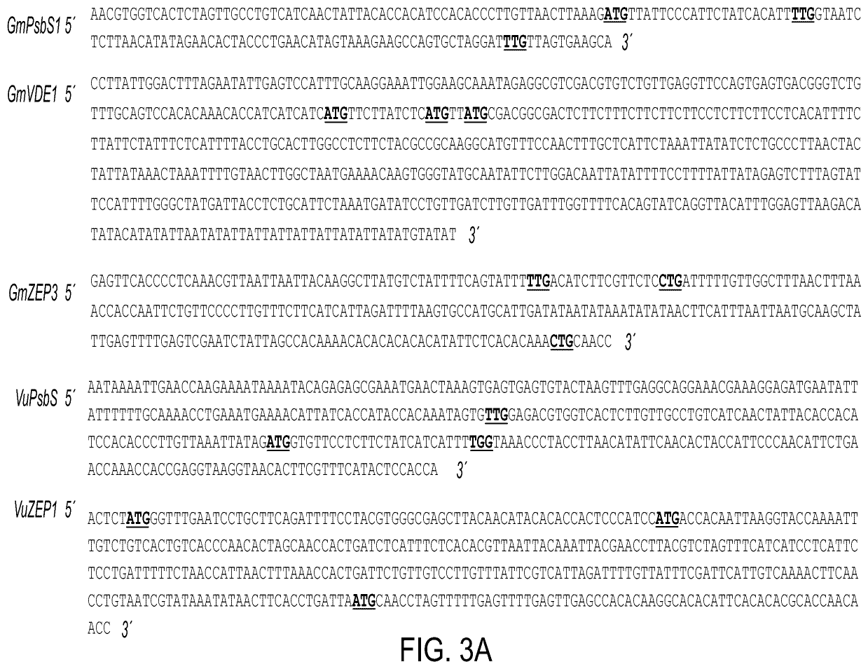

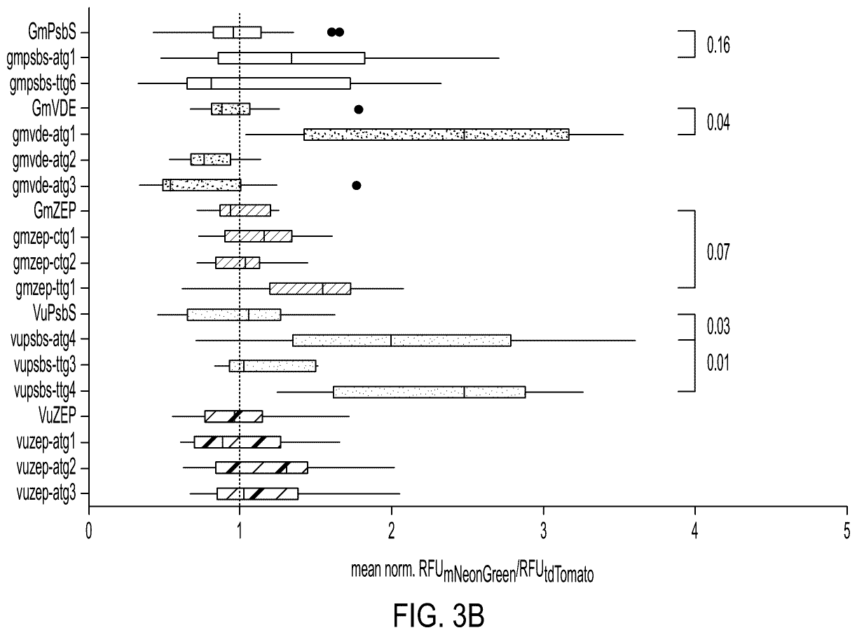

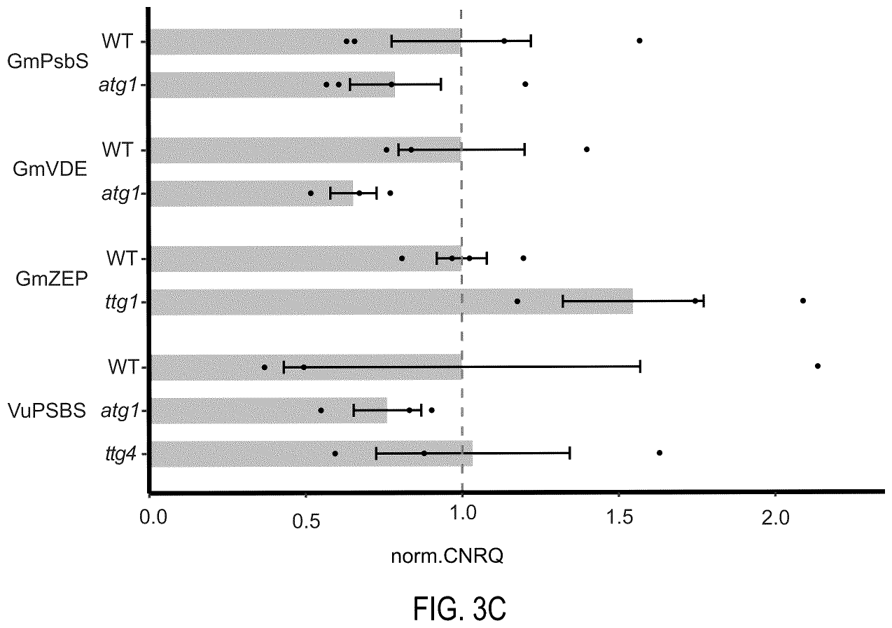

Of the two Glycine max PsbS genes, GmPsbS1 (Glyma.04G249700) was selected for testing as protein blot analysis showed that it had the highest expression (data not shown). The GmPsbS1 TLS contained 9 canonical and non-canonical uORFs, two of which were selected for testing (FIG. 3A). Multiple constructs were prepared including GmPsbS1, a construct containing all functional GmPsbS1 uORFs (insert sequence was SEQ ID NO: 1); gmpsbs1-atg1, a construct containing a mutated GmPsbS1 ATG1 uORF (insert sequence was SEQ ID NO: 2); and gmpsbs1-ttg6, a construct containing a mutated GmPsbS1 TTG6 uORF (insert sequence was SEQ ID NO: 3). Mutation of uORFs was done by replacing the start codon of the uORF with an AAA nucleotide triple. In addition, a construct was prepared containing all mutated GmPsbS1 uORFs (gmpsbs1-mut), and a construct was prepared containing a mutated GmPsbS1 TTG7 uORF (gmpsbs1-ttg7). The constructs were prepared as described in Example 1.

Producing Vigna unguiculata PsbS Constructs

In V. unguiculata, one PsbS gene (Vigun09g165900; VuPsbS) was identified, which had two transcript isoforms. The isoform Vigun09g165900.1 was selected for testing. The VuPsbS TLS was found to be 340 bp in length and to possess a large number of uORFs. Of these uORFs, three were selected for testing (FIG. 3A), namely an uORF with start codon ATG (designated ATG4), and two uORFs with start codon TTG (designated TTG3 and TTG4). None of the targeted uORFs overlapped with the primary open reading frame (mORF) for the VuPsbS gene, and the two TTG uORFs were in-frame of each other. One construct was produced with targeted mutations in ATG4, designated vupsbs-atg4 (insert sequence was SEQ ID NO: 13), a second construct was produced with targeted mutations in TTG3, designated vupsbs-ttg3 (insert sequence was SEQ ID NO: 14), and a third construct was produced with targeted mutations in TTG4, designated vupsbs-ttg4 (insert sequence was SEQ ID NO: 15). In addition to the constructs with mutations in the uORFs, a construct was prepared with the wild-type TLS, designated VuPsbS (insert sequence was SEQ ID NO: 12). The constructs were prepared as described in Example 1.

### Transient Reporter Assay

The transient reporter assay was conducted and analyzed as described in Example 1. The sample size for each construct was n=8-12, indicating eight to twelve independently, transiently transformed N. benthamiana plants (1 leaf disc per transformation).

Relative mRNA Level Determination Via RT-qPCR

The samples transformed with the GmPsbS1, gmpsbs-atg1, and gmpsbs-ttg6 constructs were processed at step 2 of the process shown in FIG. 1 for RT-qPCR to determine relative mRNA level. Primers were used to amplify the target mNeonGreen and reference PP2A (N. benthamiana, NCBI: TC21939). Normalized relative quantities (NRQ) and significance by ANOVA were calculated using qBase+ software, version 3.2 (https://doi.org/10.1186/gb-2007-8-2-r19) using target specific amplification efficiencies, normalization to reference gene, and mean Cq of three technical replicates excluding replicates with a greater than 0.5 Cq difference. The sample size for ANOVA testing was n=3-4, indicating three to four independently, transiently transformed N. benthamiana plants (1 leaf disc per transformation).

### Results

### GmPsbS1

Comparisons of GmPsbS1 constructs with different mutated uORFs to the GmPsbS1 with the wild-type leader sequence are shown in FIG. 3B. Although the comparison of gmpsbs-atg1 with GmPsbS was not found to be statistically significant (p=0.16), both constructs with mutated uORFs showed an increase of expression when compared to GmPsbS1.

### VuPsbS

The results of comparing different mutated uORFs of VuPsbS with the wild-type leader sequence are also depicted in FIG. 3B. Both the vupsbs-atg4 and the vupsbs-ttg4 constructs resulted in significant overexpression (p=0.03 and p=0.01, respectively) as compared to the VuPsbS construct. In contrast, the vupsbs-ttg3 construct did not show a difference in expression, indicating that this uORF was not a promising candidate for further expression testing.

These results demonstrated that PsbS uORFs from multiple plant species could be mutated to achieve expression differences.

### Example 3: Targeted Editing of uORFs of Glycine max VDE

The following example describes the editing of uORFs of Glycine max VDE. VDE is a gene involved in non-photochemical quenching (NPQ), where excess light energy is dissipated as heat, and editing VDE uORFs to overexpress VDE had the potential to accelerate plant recovery from NPQ. Improving the rate of NPQ recovery has been shown to increase biomass yield over the course of a field season in important crop plants.

### Materials and Methods

### Producing Glycine max VDE Constructs

Of the two Glycine max VDE genes, GmVDE1 (Glyma.19G251000) was selected for testing as protein blot analysis showed that it had the highest expression (data not shown). Three of the GmVDE1 uORFs were selected for testing (FIG. 3A). Multiple constructs were prepared including GmVDE, a construct containing all functional GmVDE1 uORFs (insert sequence was SEQ ID NO: 4); gmvde1-atg1, a construct containing a mutated GmVDE1 ATG1 uORF (insert sequence was SEQ ID NO: 5); gmvde1-atg2, a construct containing a mutated GmVDE1 ATG2 uORF (insert sequence was SEQ ID NO: 6); and gmvde1-atg3, a construct containing a mutated GmVDE1 ATG3 uORF (insert sequence was SEQ ID NO: 7). Mutation of uORFs was done by replacing the start codon of the uORF with an AAA nucleotide triplet. In addition, a construct was prepared containing all mutated GmVDE1 uORFs (gmvde1-mut). The constructs were prepared as described in Example 1.

### Transient Reporter Assay

The transient reporter assay was conducted and analyzed as described in Example 1. The sample size for each construct was n=8-12, indicating eight to twelve independently, transiently transformed N. benthamiana plants (1 leaf disc per transformation). Adjusted p-values were indicated for mutations resulting in signal increases of >40%,

Relative mRNA Level Determination Via RT-qPCR

The samples transformed with the GmVDE and gmvde1-atg1 constructs were processed at step 2 of the process shown in FIG. 1 for RT-qPCR to determine relative mRNA level. Primers were used to amplify the target mNeonGreen and reference PP2A (N. benthamiana, NCBI: TC21939). Normalized relative quantities (NRQ) and significance by ANOVA were calculated using qBase+ software, version 3.2 (https://doi.org/10.1186/gb-2007-8-2-r19) using target specific amplification efficiencies, normalization to reference gene, and mean Cq of three technical replicates excluding replicates with a greater than 0.5 Cq difference. The sample size for each construct was n=3-4, indicating three to four independently, transiently transformed N. benthamiana plants.

### Results

The results of comparing the functional and various mutated uORFs of GmVDE1 are depicted in FIG. 3A. The construct designated gmvde-atg1 exhibited significant overexpression as compared to GmVDE (p=0.04). In contrast, gmvde-atg2 and gmvde-atg3 exhibited slightly decreased expression as compared to GmVDE.

Additionally, relative mRNA levels were compared between the GmVDE construct and the gmvde-atg1 construct, as shown in FIG. 3C. While an expression difference was observed, it was not significant. This indicated that the effect of mutating the ATG1 uORF was post-transcriptional.

These results demonstrated that mutating the ATG1 uORF was sufficient to significantly overexpress GmVDE1.

### Example 4: Targeted Editing of uORFs of ZEP Across Multiple Plant Species

The following example describes the editing of uORFs of ZEP genes across multiple plant species. ZEP is a gene involved in non-photochemical quenching (NPQ), where excess light energy is dissipated as heat, and editing ZEP uORFs to overexpress ZEP had the potential to accelerate plant recovery from NPQ. Improving the rate of NPQ recovery has been shown to increase biomass yield over the course of a field season in important crop plants.

### Materials and Methods

Identifying ZEP Targets for uORF Constructs

To identify ZEP gene targets for uORF constructs, the tool Phytozome (phytozome-next[dot]jgi[dot]doe[dot]gov) was used. Zeaxanthin epoxidase (ZEP) was selected for testing, due to its importance for non-photochemical quenching. ZEP converts zeaxanthin into antheraxanthin in the photoprotective xanthophyll cycle. ZEP genes in Glycine max (soybean) and Vigna unguiculata (cowpea) were selected for testing. Specifically, the soybean gene GmZEP3 (Glyma.17G174500) and the cowpea gene VuZEP1 (Vigun03g277500) were selected.

### Producing Glycine max ZEP Constructs

Of the three G. max ZEP genes, GmZEP3 (Glyma.17G174500) was selected for testing as protein blot analysis showed that it had the highest expression (data not shown). Three GmZEP3 uORFs were selected for testing (FIG. 3A). Multiple constructs were prepared including GmZEP, a construct containing all functional GmZEP3 uORFs (insert sequence was SEQ ID NO: 8); gmzep-ctg1, a construct containing a mutated GmZEP3 CTG1 uORF (insert sequence was SEQ ID NO: 9); gmzep-ctg2, a construct containing a mutated GmZEP3 CTG2 uORF (insert sequence was SEQ ID NO: 10); and gmzep-ttg1, a construct containing a mutated GmZEP3 TTG1 uORF (insert sequence was SEQ ID NO: 11). Mutation of uORFs was done by replacing the start codon of the uORF with a CCC or TTT nucleotide triplet. The constructs were prepared as described in Example 1.

Producing Vigna unguiculata ZEP constructs

In V. unguiculata, three ZEP genes were identified: Vigun03g277500 (VuZEP1, five transcript isoforms); Vigun02g143900 (VuZEP2, five transcript isoforms); Vigun11g002300.1 (VuZEP3, least homology with the other two, likely lutein epoxidase activity, six transcript isoforms). Of the three V. unguiculata ZEP genes, VuZEP1 (Vigun03g277500) was selected for testing as protein blot analysis showed that it had the highest expression (data not shown), and of the five transcript isoforms, the Vigun03g277500.1 isoform was selected. Three VuZEP1 uORFs were selected for testing (FIG. 3A). Multiple constructs were prepared including VuZEP1, a construct containing all functional VuZEP1 uORFs (insert sequence was SEQ ID NO: 16); vuzep-atg1, a construct containing a mutated VuZEP1 ATG1 uORF (insert sequence was SEQ ID NO: 17); vuzep-atg2, a construct containing a mutated VuZEP1 ATG2 uORF (insert sequence was SEQ ID NO: 18); and vuzep-atg3, a construct containing a mutated VuZEP1 ATG3 uORF (insert sequence was SEQ ID NO: 19). Mutation of uORFs was done by replacing the start codon of the uORF with an AAA nucleotide triplet. The constructs were prepared as described in Example 1.

### Transient Reporter Assay

The transient reporter assay was conducted and analyzed as described in Example 1. The sample size for each construct was n=8-12, indicating eight to twelve independently, transiently transformed N. benthamiana plants (1 leaf disc per transformation).

Relative mRNA Level Determination Via RT-qPCR

The samples transformed with the constructs were sampled and analyzed for RT-qPCR as in Examples 2 and 3.

### Results

The results of comparing the functional and various mutated uORFs of GmZEP3 are depicted in FIG. 3B. The gmzep-ttg1 construct showed significant overexpression when compared to the GmZEP construct (p=0.07). In contrast, the gmzep-ctg1 and gmzep-ctg2 constructs showed comparable expression to the GmZEP construct.

The results of comparing different mutated uORFs of VuZEP1 with the wild-type leader sequence are also depicted in FIG. 3B. Although none showed significant differences in expression compared to the VuZEP1 construct, the vuzep-atg2 and vuzep-atg3 constructs showed slightly increased expression.

These results demonstrated that VDE uORFs from multiple plant species could be mutated to achieve expression differences.

### Example 5: Targeted Editing of uORFs of Vigna unguiculata VDE

The following example describes the editing of uORFs of Vigna unguiculata VDE.

### Materials and Methods

Producing Vigna unguiculata VDE Constructs

The Vigna unguiculata VDE gene (VuVDE; Vigun06g119100) is selected for testing. The mutation of uORFs and design of constructs is done as in Examples 2-4. Constructs are prepared as described in Example 1.

### Transient Reporter Assay

The transient reporter assay is conducted and analyzed as described in Example 1.

### Results

Mutating the one or more uORFs in VuVDE results in overexpression of VuVDE.

### Example 6: Use of CRISPR/Cas Systems for Stably Editing uORFs

The following example describes the stable editing of uORFs using CRISPR/Cas.

### Materials and Methods

Identifying uORF Targets

The uORF targets in TLSs of PsbS, VDE, and ZEP genes identified in examples 3-5 are selected for editing. Additional uORF targets in TLSs of PsbS, VDE, and ZEP genes in other plant species may also be selected for editing.

Editing uORF Targets

CRISPR/Cas constructs are generated that target the identified uORF targets. Target plants are selected for editing, and CRISPR/Cas constructs are cloned. Expression vectors are produced containing these constructs, and vectors are then transformed into Agrobacterium for infiltration or are otherwise delivered to plant tissue. The CRISPR/Cas system edits the targeted TLSs.

One, multiple, or all uORFs for a target gene are edited. Multiplex editing allows the opportunity of targeting many uORFs, many genes, or many rounds of targeting gene(s).

### Results

Plants with stably edited uORFs in PsbS, VDE, and ZEP genes are produced.

### Example 7: Use of CRISPR/Cas Systems for Stably Editing uORFs

The following example describes the stable multiplex editing of multiple Glycine max uORFs using CRISPR-Cas9. Two constructs were generated: one plasmid was referred to as “RC00531” and contained targets for two uORFs, each targeting a NPQ gene; the other plasmid was referred to as “RC00530” and contained targets for three uORFs, each targeting a NPQ gene. Editing with these constructs results in a double knockout line of Glycine max referred to as GmVZ and a triple knockout line of Glycine max referred to as GmVPZ.

### Materials and Methods

Identifying uORF Targets

An uORF target in TLSs of PsbS, VDE, and ZEP genes were identified as in examples 3-6, and these were selected for editing in Glycine max. GmPsbS is also referred to as Glyma.04G249700 (SEQ ID NO: 97), GmVDE is also referred to as Glyma.19G251000 (SEQ ID NO: 99), and GmZEP is also referred to as Glyma.17G174500 (SEQ ID NO: 98).

Editing uORF Targets

CRISPR/Cas constructs were generated that targeted the identified uORF targets, as described in Example 6. CRISPR/Cas constructs were cloned using the publicly available plasmid “pDIRECT_23C” (Cermak et al. 2017) and specific combinations of sgRNAs.

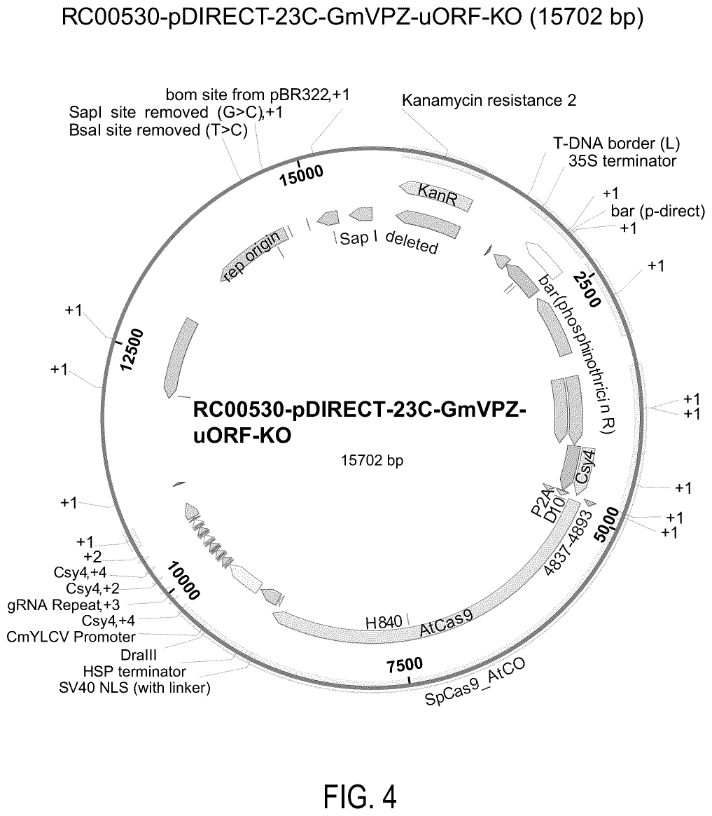

Construct RC00530 included the full-length gRNAs displayed in Table 2, with two sgRNAs flanking each uORF, totaling 6 gRNAs. This construct's plasmid is shown in FIG. 4.

TABLE 2
Full-length gRNAs used in construct RC00530

| | | SEQ ID |
|---|---|---|
| gRNA ID | Sequence | NO: |
| gmzep-sg1 | GTAATTAATTAACGTTTGAGGTTTTAGAGCTA | 89 |
| | GAAATAGCAAGTTAAAATAAGGCTAGTCCGT | |
| | TATCAACTTGAAAAAGTGGCACCGAGTCGGTGC | |
| gmzep-sg2 | AACAAGGGGAACAGAATTGGGTTTTAGAGCT | 90 |
| | AGAAATAGCAAGTTAAAATAAGGCTAGTCCG | |
| | TTATCAACTTGAAAAAGTGGCACCGAGTCGGTGC | |
| gmvde-sg1 | CAAACAGACCCGTCACTCACGTTTTAGAGCT | 91 |
| | AGAAATAGCAAGTTAAAATAAGGCTAGTCC | |
| | GTTATCAACTTGAAAAAGTGGCACCGAGTCGGTGC | |
| gmvde-sg2 | TCTTATCTCATGTTATGCGAGTTTTAGAGCTA | 92 |
| | GAAATAGCAAGTTAAAATAAGGCTAGTCCGT | |
| | TATCAACTTGAAAAAGTGGCACCGAGTCGGTGC | |
| gmpsbs-sg1 | ATAACATCTTTAAGTTAACAGTTTTAGAGCTA | 93 |
| | GAAATAGCAAGTTAAAATAAGGCTAGTCCGTT | |
| | ATCAACTTGAAAAAGTGGCACCGAGTCGGTGC | |
| gmpsbs-sg2 | ATTCCCATTCTATCACATTTGTTTTAGAGCTAG | 94 |
| | AAATAGCAAGTTAAAATAAGGCTAGTCCGTTA | |
| | TCAACTTGAAAAAGTGGCACCGAGTCGGTGC | |

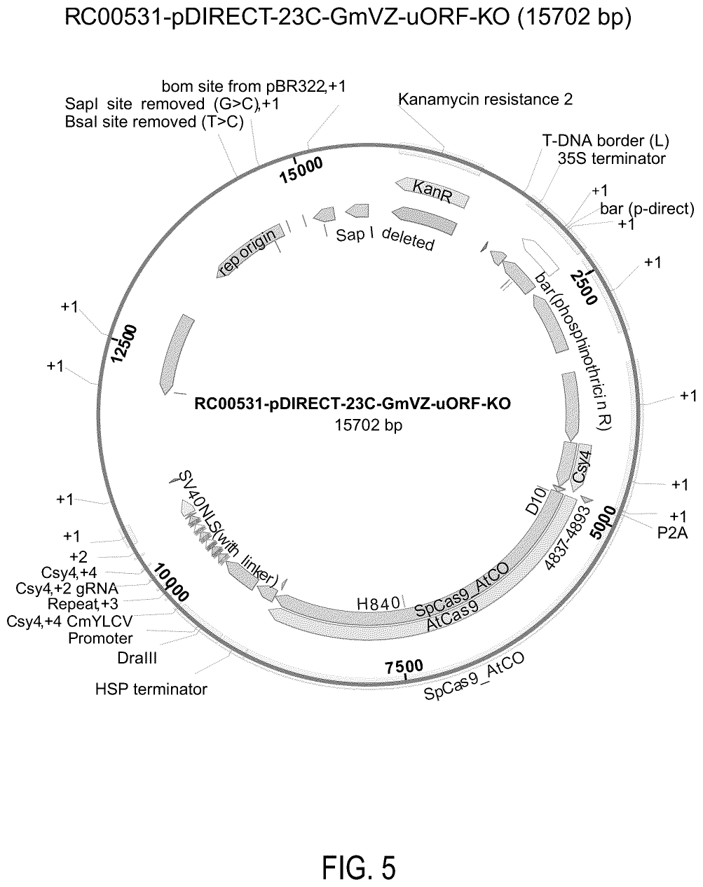

Construct RC00531 included the full-length gRNAs displayed in Table 3, with three sgRNAs flanking each uORF, totaling 6 gRNAs. This construct's plasmid is shown in FIG. 5.

TABLE 3
Full-length gRNAs used in construct RC00531

| | | SEQ |
|---|---|---|
| | | ID |
| gRNA ID | Sequence | NO: |
| gmzep-sg1 | GTAATTAATTAACGTTTGAGGTTTTAGAGCTA | 89 |
| | GAAATAGCAAGTTAAAATAAGGCTAGTCCGT | |
| | TATCAACTTGAAAAAGTGGCACCGAGTCGGTGC | |
| gmzep-sg2 | AACAAGGGGAACAGAATTGGGTTTTAGAGCT | 90 |
| | AGAAATAGCAAGTTAAAATAAGGCTAGTCCG | |
| | TTATCAACTTGAAAAAGTGGCACCGAGTCGGTGC | |
| gmzep-sg3 | AGTTAAAGCCAACAAAAATCGTTTTAGAGCT | 95 |
| | AGAAATAGCAAGTTAAAATAAGGCTAGTCCG | |
| | TTATCAACTTGAAAAAGTGGCACCGAGTCGGTGC | |
| gmvde-sg1 | TCTTATCTCATGTTATGCGAGTTTTAGAGCTA | 91 |
| | GAAATAGCAAGTTAAAATAAGGCTAGTCCGT | |
| | TATCAACTTGAAAAAGTGGCACCGAGTCGGTGC | |
| gmvde-sg2 | ATAACATCTTTAAGTTAACAGTTTTAGAGCTA | 92 |
| | GAAATAGCAAGTTAAAATAAGGCTAGTCCGTT | |
| | ATCAACTTGAAAAAGTGGCACCGAGTCGGTGC | |
| gmvde-sg3 | GCGTAGAAGAGGCCAAGTGCGTTTTAGAGCTA | 96 |
| | GAAATAGCAAGTTAAAATAAGGCTAGTCCGTT | |
| | ATCAACTTGAAAAAGTGGCACCGAGTCGGTGC | |

The uORF disruption through editing was designed around the following deletion products, which remove each uORF's start codon by induction of two double-stranded breaks flanking the start codon. Editing uORFs of GmPsbS were designed around the transcript leader of Glyma.04G249700, which is available through Soybase (Brown et al. 2021. A new decade and new data at SoyBase, the USDA-ARS soybean genetics and genomics database. Nucl. Acids Res. 49 (D1): D1496-D15012) at www[dot]soybase[dot]org/sbt/search/search_results.php?category=FeatureName&version=G lyma2[dot]0&search_term=Glyma[dot]04g249700 to create a deletion product ΔGlyma.04G249700 (GmPsbS-del), or other analogous mutations affecting the described uORFs. Editing uORFs of GmVDE were designed around the transcript leader of Glyma.19G251000, which is available through Soybase at www[dot]soybase[dot]org/sbt/search/search_results[dot]php?category=FeatureName&versio n=Wm82[dot]a4[dot]v1&search_term=Glyma[dot]19g251000 to create a deletion product ΔGlyma.19G251000 (GmVDE-del), or other analogous mutations affecting the described uORFs. Editing uORFs of GmZEP were designed around the transcript leader of Glyma.17G174500, which is available through Soybase at www[dot]soybase[dot]org/sbt/search/search_results[dot]php?category=FeatureName&versio n=Wm82[dot]a4[dot]v1&search_term=Glyma[dot]17g174500 to create a deletion product ΔGlyma.17G174500 (GmZEP-del), or other analogous mutations affecting the described uORFs. Prophetic in planta deletion products GmPsbS-del has the sequence of SEQ ID NO: 100, GmVDE-del has the sequence of SEQ ID NO: 102, and GmZEP-del has the sequence of SEQ ID NO: 101.

Expression vectors are produced containing these constructs, and vectors are then transformed into Agrobacterium for infiltration or are otherwise delivered to plant tissue. The CRISPR/Cas system edits the targeted TLSs.

### Results

Plants with stably edited uORF knockouts in uORFs of GmPsbS, GmVDE, and GmZEP show greater protein activity of PsbS, VDE, and ZEP than wild-type plants. Plants with stably edited uORF knockouts in uORFs of GmVDE and GmZEP show greater protein activity of VDE and ZEP than wild-type plants. Through editing the uORFs, the expression of these NPQ genes is increased, which results in increased efficiency of photosynthesis during sun-shade transitions and ultimately biomass accumulation. It is predicted that the triple uORF knockout line (GmVPZ) has a greater effect on the amplitude of NPQ compared to the double uORF knockout line (GmVZ), which is expected to affect qZ and qE. However, it is predicted that GmVZ could result in greater reduction of max NPQ, which could be compensated in the GmVPZ approach.

What is claimed is:

1. A genetically modified plant comprising one or more edited endogenous nucleotide sequences encoding a photosystem II subunit S (PsbS) polypeptide, a zeaxanthin epoxidase (ZEP) polypeptide, or a violaxanthin de-epoxidase (VDE) polypeptide, wherein the one or more edited nucleotide sequences are edited at one or more upstream open reading frames (uORFs) in a transcript leader sequence (TLS).

2. The genetically modified plant of claim 1, wherein the edits at one or more uORFs increase expression of the downstream main open reading frame (mORF) encoding the PsbS polypeptide, the ZEP polypeptide, and/or the VDE polypeptide, and/or wherein the edited endogenous nucleotide sequences were edited at two or more, three or more, four or more, five or more, six or more, seven or more, eight or more, or nine uORFs in a TLS.

3. The genetically modified plant of claim 1, wherein
(i) the editing removed uORF repression, increased efficiency of mORF translation re-initiation, removed stalling of a ribosome, removed inhibition of translation of downstream ORFs, or increased translational efficiency of downstream ORFs;
(ii) the editing resulted in disruption of an uORF start codon, and wherein disruption was achieved by replacement of the uORF start codon with a nucleotide triplet, deletion of the third base of the uORF start codon, a frameshift mutation to disrupt an uORF reading frame, insertion of one or more nucleotides in the uORF, or deletion of one or more nucleotides in the uORF;
(iii) uORF repression is reduced or removed, efficiency of mORF translation re-initiation is increased, stalling of a ribosome is reduced or removed, inhibition of translation of downstream ORFs is reduced or removed, or translational efficiency of downstream ORFs is increased;
(iv) an uORF start codon is disrupted; and/or
(v) the uORF start codon was replaced with a nucleotide triplet, deletion of the third base of the uORF start codon, a frameshift mutation to disrupt an uORF reading frame, insertion of one or more nucleotides in the uORF, or deletion of one or more nucleotides in the uORF.

4. The genetically modified plant of claim 1, comprising
(i) edited endogenous nucleotide sequences encoding the ZEP polypeptide and the VDE polypeptide or the PsbS polypeptide and the ZEP polypeptide; or
(ii) edited endogenous nucleotide sequences encoding the PsbS polypeptide, the ZEP polypeptide, and the VDE polypeptide.

5. The genetically modified plant of claim 1, comprising further genetic modifications that increase expression of one or more of the PsbS polypeptide, the ZEP polypeptide, or the VDE polypeptide.

6. The genetically modified plant of claim 5,
wherein the expression of the PsbS polypeptide is increased by expressing a transfected nucleotide sequence encoding the PsbS polypeptide in the genetically modified plant, by a genetic modification in a promoter of the edited endogenous nucleotide sequence encoding the PsbS polypeptide and/or by a genetic modification in a promoter of an endogenous nucleotide sequence encoding the PsbS polypeptide;
wherein the expression of the ZEP polypeptide is increased by expressing a transfected nucleotide sequence encoding the ZEP polypeptide in the genetically modified plant, by a genetic modification in a promoter of the edited endogenous nucleotide sequence encoding the ZEP polypeptide, and/or by a genetic modification in a promoter of an endogenous nucleotide sequence encoding the ZEP polypeptide; and/or
wherein the expression of the VDE polypeptide is increased by expressing a transfected nucleotide sequence encoding the VDE polypeptide in the genetically modified plant, by a genetic modification in a promoter of the edited endogenous nucleotide sequence encoding the VDE polypeptide, and/or by a genetic modification in a promoter of an endogenous nucleotide sequence encoding the VDE polypeptide.

7. The genetically modified plant of claim 5, wherein
(i) the expression of the PsbS polypeptide is increased by expressing the transfected nucleotide sequence encoding the PsbS polypeptide, and wherein the transfected nucleotide sequence is operably linked to at least one expression control sequence; wherein the expression of the VDE polypeptide is increased by expressing the transfected nucleotide sequence encoding the VDE polypeptide, and wherein the transfected nucleotide sequence is operably linked to at least one expression control sequence; and/or wherein the expression of the ZEP polypeptide is increased by expressing the transfected nucleotide sequence encoding the ZEP polypeptide, and wherein the transfected nucleotide sequence is operably linked to at least one expression control sequence; and/or
(ii) wherein the one or more edited endogenous nucleotide sequences encodes the ZEP polypeptide, and wherein the expression of the VDE polypeptide is increased by expressing the transfected nucleotide sequence encoding the VDE polypeptide; wherein the one or more edited endogenous nucleotide sequences encodes the VDE polypeptide, and wherein the expression of the ZEP polypeptide is increased by expressing the transfected nucleotide sequence encoding the ZEP polypeptide; wherein the one or more edited endogenous nucleotide sequences encodes the PsbS polypeptide, and wherein the expression of the ZEP polypeptide is increased by expressing the transfected nucleotide sequence encoding the ZEP polypeptide; wherein the one or more edited endogenous nucleotide sequences encodes the ZEP polypeptide, and wherein the expression of the PsbS polypeptide is increased by expressing the transfected nucleotide sequence encoding the PsbS polypeptide.

8. The genetically modified plant of claim 2, wherein
(i) the expression of both the ZEP polypeptide and the VDE polypeptide are increased under the same conditions as compared to a control plant or the expression of both the PsbS polypeptide and the ZEP polypeptide are increased as compared to a control plant grown under the same conditions; or
(ii) wherein the expression of all three of the ZEP polypeptide, the PsbS polypeptide, and the VDE polypeptide are increased as compared to a control plant grown under the same conditions,
wherein the conditions are fluctuating light conditions.

9. The genetically modified plant of claim 1, wherein the editing was done using a gene editing technique selected from a transcription activator-like effector nuclease (TALEN) gene editing technique, a clustered Regularly Interspaced Short Palindromic Repeat (CRISPR/Cas) gene editing technique, a zinc-finger nuclease (ZFN) gene editing technique, or combinations of the foregoing, optionally wherein the gene editing technique was a clustered Regularly Interspaced Short Palindromic Repeat (CRISPR/Cas) gene editing technique, and wherein the Cas enzymes were selected from the group consisting of Cas9, Cas12, Cas12a, Cas13, Cas14, CasX, and CasY.

10. The genetically modified plant of claim 1, wherein the plant is a crop plant, a model plant, a monocotyledonous plant, a dicotyledonous plant, a plant with Crassulacean acid metabolism (CAM) photosynthesis, a plant with C3 photosynthesis, a plant with C4 photosynthesis, an annual plant, a greenhouse plant, a horticultural flowering plant, a perennial plant, a switchgrass plant, a maize plant, a biomass plant, an Arabidopsis thaliana plant, a tobacco (Nicotiana tabacum) plant, a rice (Oryza sativa) plant, a corn (Zea mays) plant, a sorghum (Sorghum bicolor) (sweet sorghum or grain sorghum) plant, a soybean (Glycine max) plant, a cowpea (Vigna unguiculata) plant, a poplar (Populus spp.) plant, a eucalyptus (Eucalyptus spp.) plant, a cassava (Manihot esculenta) plant, a barley (Hordeum vulgare) plant, a potato (Solanum tuberosum) plant, a sugarcane (Saccharum spp.) plant, an alfalfa (Medicago sativa) plant, a Miscanthus plant, an energy cane plant, an elephant grass plant, a wheat plant, an oat plant, an oil palm plant, a safflower plant, a sesame plant, a flax plant, a cotton plant, a sunflower plant, a Camelina plant, a Brassica napus plant, a Brassica carinata plant, a Brassica juncea plant, a pearl millet plant, a foxtail millet plant, an other grain plant, an oilseed plant, a vegetable crop plant, a forage crop plant, an industrial crop plant, or a woody crop plant, optionally wherein the plant is a soybean (Glycine max) plant or a cowpea (Vigna unguiculata) plant.

11. The genetically modified plant of claim 1, wherein:
the plant has improved growth under fluctuating light conditions as compared to the control plant grown under the same fluctuating light conditions;
the plant has increased lutein under fluctuating light conditions as compared to the control plant grown under the same fluctuating light conditions;
the plant has increased photosynthetic efficiency under fluctuating light conditions as compared to the control plant grown under the same fluctuating light conditions;
the plant has improved photoprotection efficiency under fluctuating light conditions as compared to the control plant grown under the same fluctuating light conditions;
the plant has an increased rate of relaxation of non-photochemical quenching (NPQ) under fluctuating light conditions as compared to the control plant grown under the same fluctuating light conditions; and/or
the plant has improved quantum yield and CO₂ fixation under fluctuating light conditions as compared to the control plant grown under the same fluctuating light conditions.

12. A plant part, plant cell, or seed of the genetically modified plant of claim 1.

13. A method of producing the genetically modified plant of claim 1, comprising:
a) providing a plant, plant part, plant cell, tissue, or other explant comprising an endogenous PsbS gene, an endogenous ZEP gene, and/or an endogenous VDE gene;
b) selecting one or more uORFs in a TLS of the endogenous PsbS gene, the endogenous ZEP gene, and/or the endogenous VDE gene for editing; and
c) using a gene editing technique to edit the one or more uORFs of the endogenous PsbS gene, the endogenous ZEP gene, and/or the endogenous VDE gene in the plant, plant part, plant cell, tissue, or other explant, and optionally regenerating the plant cell, tissue, or other explant into a genetically altered plantlet that is grown into a plant, to produce a genetically modified plant comprising one or more edited PsbS, ZEP, and/or VDE genes.

14. The method of claim 13, optionally further comprising increasing expression of one or more of the PsbS polypeptide, the VDE polypeptide, or the ZEP polypeptide before step (c) or in step (c),
wherein the expression of the PsbS polypeptide is increased by expressing a transfected nucleotide sequence encoding the PsbS polypeptide in the genetically modified plant, by a genetic modification in a promoter of the edited endogenous nucleotide sequence encoding the PsbS polypeptide and/or by a genetic modification in a promoter of an endogenous nucleotide sequence encoding the PsbS polypeptide;
wherein the expression of the ZEP polypeptide is increased by expressing a transfected nucleotide sequence encoding the ZEP polypeptide in the genetically modified plant, by a genetic modification in a promoter of the edited endogenous nucleotide sequence encoding the ZEP polypeptide, and/or by a genetic modification in a promoter of an endogenous nucleotide sequence encoding the ZEP polypeptide; and/or
wherein the expression of the VDE polypeptide is increased by expressing a transfected nucleotide sequence encoding the VDE polypeptide in the genetically modified plant, by a genetic modification in a promoter of the edited endogenous nucleotide sequence encoding the VDE polypeptide, and/or by a genetic modification in a promoter of an endogenous nucleotide sequence encoding the VDE polypeptide.

15. The method of claim 13, wherein the editing is done using a gene editing technique selected from a transcription activator-like effector nuclease (TALEN) gene editing technique, a clustered Regularly Interspaced Short Palindromic Repeat (CRISPR/Cas) gene editing technique, a zinc-finger nuclease (ZFN) gene editing technique, or combinations of the foregoing, optionally wherein the gene editing technique is a clustered Regularly Interspaced Short Palindromic Repeat (CRISPR/Cas) gene editing technique, and/or wherein the Cas enzymes are selected from the group consisting of Cas9, Cas12, Cas12a, Cas13, Cas14, CasX, and CasY.

16. An expression vector or isolated DNA molecule comprising one or more gene editing components that target one or more uORF sequences of an endogenous PsbS gene, wherein the uORF sequence is edited by the one or more gene editing components to remove uORF translational repression; comprising one or more gene editing components that target one or more uORF sequences of an endogenous VDE gene, wherein the uORF sequence is edited by the one or more gene editing components to remove uORF translational repression; and/or comprising one or more gene editing component that target one or more uORF sequences of an endogenous ZEP gene, wherein the uORF sequence is edited by the one or more gene editing components to remove uORF translational repression, optionally
wherein the one or more gene editing components are selected from the group consisting of a ribonucleoprotein complex that targets the one or more uORF sequences; a vector comprising a TALEN protein encoding sequence, wherein the TALEN protein targets the one or more uORF sequences; a vector comprising a ZFN protein encoding sequence, wherein the ZFN protein targets the one or more uORF sequences; an oligonucleotide donor (ODN), wherein the ODN targets the one or more uORF sequences; and a vector comprising a CRISPR/Cas enzyme encoding sequence and a targeting sequence, wherein the targeting sequence targets the one or more uORF sequences.

17. A bacterial cell or an Agrobacterium cell comprising the expression vector or isolated DNA molecule of claim 16.

18. A genetically modified plant, plant part, plant cell, or seed including the expression vector or isolated DNA molecule of claim 16.

19. A composition or kit comprising the expression vector or isolated DNA molecule of claim 16 with a bacterial cell or an Agrobacterium cell.

20. A method of increasing the rate of relaxation of NPQ in a plant, comprising:
introducing a genetic alteration via the expression vector or isolated DNA molecule of claim 16 to a cell, optionally wherein the cell is a plant cell.

21. A transient screening method to measure gene expression, comprising:
a) providing one or more dual-fluorescence expression vectors comprising a test insert to produce one or more test vectors and at least one dual-fluorescence expression vector comprising a control insert to produce at least one control vector, wherein the dual-fluorescence expression vectors comprise a first fluorescent reporter and a second fluorescent reporter;
b) introducing the one or more test vectors into a first leaf tissue and introducing the at least one control vector into a second leaf tissue, wherein the first leaf tissue and the second leaf tissue are of the same plant variety;
d) excising leaf portions from the first leaf tissue and from the second leaf tissue 3 days post injection;
e) placing the leaf portions abaxially in a multiwell plate; and
f) measuring fluorescence on a monochromator-based plate reader in two separate channels, wherein a first channel is used to obtain a signal from the first fluorescent reporter and a second channel is used to obtain a signal from the second fluorescent reporter.

22. The transient screening method of claim 21, wherein step b) comprises:
b-1) transforming a first Agrobacterium tumefaciens strain with the one or more test vectors and transforming a second A. tumefaciens strain with the at least one control vector; and
b-2) injecting the first A. tumefaciens strain harboring the one or more test vectors into the first leaf tissue and injecting the second A. tumefaciens strain into the second leaf tissue.

23. The transient screening method of claim 21, wherein
(a) the leaf portions are discs;
(b) the multiwall plate is a 96-well plate or a 384-well plate; and/or
(c) wherein the first fluorescent reporter is a green fluorescent reporter and the second fluorescent reporter is a red fluorescent reporter, optionally wherein the first fluorescent reporter is mNeonGreen and the second fluorescent reporter is tdTomato.

24. The transient screening method of claim 21, further comprising step (g) normalizing the signal obtained from the first fluorescent reporter in step (f) to the signal obtained from the second fluorescent reporter in step (f) to reduce noise, and to produce a graph of normalized results comparing results obtained from the one or more test vectors with results obtained from the at least one control vector to determine expression differences between the one or more test vectors and the at least one control vector, optionally wherein comparing results further comprises determining p-values using a nonparametric bootstrap for two-sample t-testing that is adjusted for multiple testing.

25. The transient screening method of claim 21, wherein the test insert comprises a mutated TLS, optionally wherein the mutated TLS is mutated in one or more uORFs, and wherein the control insert comprises a wild-type TLS.
